(19) United States
(12) Patent Application Publication
(10) Pub. No.: US 2021/0123016 A1
(43) Pub. Date: Apr. 29, 2021

(54) REGULATORS OF HUMAN PLURIPOTENT STEM CELLS AND USES THEREOF

(71) Applicants: Robert John Ihry, Cambridge, MA (US); Ajamete Kaykas, Cambridge, MA (US); Marc Horst Peter Hild, Cambridge, MA (US); Novartis AG, Basel (CH)

(72) Inventors: Robert John Ihry, Arlington, MA (US); Ajamete Kaykas, Cambridge, MA (US); Marc Horst Peter Hild, Wellesley, MA (US)

(21) Appl. No.: 17/052,041

(22) PCT Filed: May 1, 2019
(86) PCT No.: PCT/US2019/030223

§ 371 Date: Oct. 30, 2020

Related U.S. Application Data
(60) Provisional application No. 62/665,834, filed on May 2, 2018.

(51) Int. Cl. C12N 5/0735; C12N 5/074

(52) U.S. Cl. CPC C12N 5/0606; C12N 2501/65; C12N 5/0696

(57) ABSTRACT

Disclosed herein are regulators, e.g., inhibitors or promoters, of human pluripotent stem cells (hPSCs), and methods of using the same. Also provided herein are methods of manufacturing hPSCs, and methods of modifying hPSCs comprising contacting the hPSCs with the regulators, e.g., inhibitors or promoters, of hPSCs, and uses thereof.

### CROSS-REFERENCE TO RELATED APPLICATIONS

This application claims the benefit of U.S. Provisional Application 62/665,834 filed on May 2, 2018, the entire contents of which is hereby incorporated by reference.

### BACKGROUND

Dissociation-induced death is a phenomenon in which human pluripotent stem cells (hPSCs) undergo cell death (e.g., apoptosis), upon dissociation into single cells (e.g., dissociation (e.g., breaking up) of cell colonies comprising hPSCs). Dissociation-induced death presents a challenge in culturing (e.g., manufacturing), and expanding hPSCs, and thus hinders the development of therapies comprising hPSCs. Provided herein are improved methods of culturing (e.g., manufacturing) and modifying hPSCs, and uses thereof.

### SUMMARY

The present disclosure provides, inter alia, regulators, e.g., inhibitors or promoters, of human pluripotent stem cells (hPSCs), including but not limited to:

(i) inhibitors of dissociation-induced death (DID), e.g., inhibitors of DID activators, e.g., inhibitors of a DID activator described in Table 7);

(ii) inhibitors of stem cell pluripotency (e.g., inhibitors of one or more targets identified as OCT4 high as described in Table 8);

(iii) promoters of stem cell pluripotency (e.g., activators of one or more targets identified as OCT4 low as described in Table 8);

(iv) inhibitor of hPSC viability, e.g., cell division, e.g., self-renewal (e.g., inhibitors of one or more targets identified as an enriched hPSC fitness gene as described in Table 4); or

(v) an activator of stem cell viability, e.g., cell division, e.g., self-renewal, e.g., one or more activators of a target identified as a depleted hPSC fitness gene as described in Table 4;

and methods of using the same.

Without wishing to be bound by theory, hPSCs are usually primed and organized in a polarized epithelium. Upon dissociation, e.g., single cell dissociation, hPSCs tend to undergo cell death; this phenomenon is referred to herein as “dissociation-induced death (DID).” Dissociation-induced death has presented challenges to the maintenance and/or genetic manipulation of hPSCs. For example, DID limits scalability and single cell cloning of hPSCs. In some embodiments described herein, loss of PRKC apoptosis WT1 regulator (PAWR) (an apoptosis regulator) resulted in inhibition of DID in hPSCs. Accordingly, provided herein are, inter alia, methods of inhibiting DID in hPSCs with a PAWR inhibitor, e.g., a PAWR inhibitor described herein. Also provided herein are methods of culturing, e.g., manufacturing, hPSCs, and methods of modifying hPSCs comprising contacting the hPSCs with a PAWR inhibitor described herein, and uses thereof. Additionally disclosed herein are methods of screening for a candidate degron using a fusion protein comprising PAWR molecule (e.g., a full length, a fragment or a functional variant of PAWR) and a degron.

Method of Culturing hPSCs

In one aspect, disclosed herein is a method of culturing, e.g., manufacturing, human pluripotent stem cells (hPSCs). The method includes:

providing human pluripotent stem cells (hPSCs) (e.g., a cell population comprising hPSCs), e.g., human embryonic stem cells (hESCs) or induced pluripotent stem cells (iPSCs);

maintaining the hPSCs under conditions that allow for maintenance of pluripotency of the hPSCs;

contacting the hPSCs, with one, two, three or more (e.g., all) of:

(i) an inhibitor of dissociation-induced death (DID), e.g., an inhibitor of a DID activator, e.g., one or more inhibitors of a DID activator disclosed in Table 7;

(ii) an inhibitor of stem cell pluripotency, e.g., one or more inhibitors of a target identified as OCT4 high as disclosed in Table 8;

(iii) a promoter of stem cell pluripotency, e.g., one or more activators of a target identified as OCT4 low as disclosed in Table 8;

(iv) an inhibitor of stem cell viability, e.g., cell divison, e.g., self-renewal, e.g., one or more inhibitors of a target identified as an enriched hPSC fitness gene as disclosed in Table 4; or

(v) an activator of stem cell viability, e.g., cell division, e.g., self-renewal, e.g., one or more activators of a target identified as a depleted hPSC fitness gene as disclosed in Table 4;

thereby culturing, e.g., manufacturing, the hPSCs.

In another aspect, the disclosure provides a method of culturing, e.g., manufacturing, human pluripotent stem cells (hPSCs), comprising:

providing human pluripotent stem cells (hPSCs) (e.g., a population of hPSCs), e.g., human embryonic stem cell (hESCs) or induced pluripotent stem cells (iPSCs);

contacting the hPSCs with with, one, two, three or more (e.g., all) of:

(i) an inhibitor of dissociation-induced death (DID), e.g., an inhibitor of a DID activator, e.g., one or more inhibitors of a DID activator disclosed in Table 7;

(ii) an inhibitor of stem cell pluripotency, e.g., one or more inhibitors of a target identified as OCT4 high as disclosed in Table 8;

(iii) a promoter of stem cell pluripotency, e.g., one or more activators of a target identified as OCT4 low as disclosed in Table 8;

(iv) an inhibitor of stem cell viability, e.g., cell divison, e.g., self-renewal, e.g., one or more inhibitors of a target identified as an enriched hPSC fitness gene as disclosed in Table 4; or

(v) an activator of stem cell viability, e.g., cell division, e.g., self-renewal, e.g., one or more activators of a target identified as a depleted hPSC fitness gene as disclosed in Table 4;

under conditions that allow for:
(a) growth of the hPSCs, e.g., about 2- to 20-fold, or e.g., about 2-, 5-, 10-, 15-, 20-fold or higher growth, e.g., as measured by an assay of Example 1;
(b) increased, e.g., preservation of, viability of the hPSCs, e.g., as measured by an assay of Example 1; or
(c) both (a) and (b);

thereby culturing, e.g., manufacturing, the hPSCs.

In some embodiments, the DID inhibitor comprises one or more of a PAWR inhibitor, or an inhibitor of a DID mediator disclosed in Table 7.

In some embodiments, the DID inhibitor comprises an inhibitor of a DID activator disclosed in Table 7. In some embodiments, the DID inhibitor is chosen from: a low molecular weight compound; an antibody molecule; an RNAi targeting (e.g., siRNA or shRNA); an epigenetic modulator of; or a genetic modulator (e.g., a nuclease, e.g., a CRISPR/Cas9, a zinc-finger nuclease (ZFN), or a Transcription activator-like effector nuclease (TALEN)).

In some embodiments, the DID inhibitor comprises a PAWR inhibitor, e.g., as described herein. In some embodiments, the PAWR inhibitor is chosen from: a low molecular weight compound inhibitor of PAWR; an anti-PAWR antibody molecule; an RNAi targeting PAWR (e.g., siRNA or shRNA); an epigenetic modulator of PAWR; or a genetic modulator of PAWR (e.g., a nuclease targeting PAWR, e.g., a CRISPR/Cas9, a zinc-finger nuclease (ZFN), or a Transcription activator-like effector nuclease (TALEN) targeting PAWR).

In some embodiments, the PAWR inhibitor, is administered at a dose that results in reduced, e.g., lesser, dissociation-induced death of hPSCs as measured by an assay of Example 1.

In some embodiments, the PAWR inhibitor, e.g., a PAWR inhibitor described herein results in one, two, three, four, five, or all (e.g., six) of the following:

i) a reduction in DID by at least 10%, 20%, 30%, 40%, 50%, 60%, 70%, 80%, 90%, 95%, 99%, or 100%, e.g., compared to the hPSCs in the absence of the PAWR inhibitor;

ii) a reduction in membrane blebbing as measured by an assay of Example 1 by at least 10%, 20%, 30%, 40%, 50%, 60%, 70%, 80%, 90%, 95%, 99%, or 100%, e.g., compared to the hPSCs in the absence of the PAWR inhibitor;

iii) an increase in one or more of survival, proliferation, expansion of the hPSCs by at least 10%, 20%, 30%, 40%, 50%, 60%, 70%, 80%, 90%, 95%, 99%, or 100%, e.g., compared to the hPSCs in the absence of the PAWR inhibitor;

iv) an increase in the ability to passage the hPSCs for a first, second, third, or more passages;

v) an increase in one or more of: survival, proliferation, or expansion of the hPSCs after one or more passages by at 10%, 20%, 30%, 40%, 50%, 60%, 70%, 80%, 90%, 95%, 99%, or 100%, e.g., compared to the hPSCs in the absence of the PAWR inhibitor; or

vi) an increase in the survival of hPSCs as single cells by at least 10%, 20%, 30%, 40%, 50%, 60%, 70%, 80%, 90%, 95%, 99%, or 100%, e.g., compared to the hPSCs in the absence of the PAWR inhibitor.

In some embodiments, the method further comprises contacting the population of cells with an additional, e.g., a second or third, DID inhibitor chosen from a Rho-dependent protein kinase (ROCK) inhibitor (e.g., a ROCK inhibitor described herein, e.g., a ROCK1 inhibitor, or a ROCK2 inhibitor, or both) or a Myosin inhibitor (e.g., a Myosin inhibitor described herein).

In some embodiments, the DID inhibitor comprises a ROCK inhibitor, e.g., Y-27632 or Thiazovivin.

In some embodiments, the DID inhibitor comprises a Myosin inhibitor, e.g., blebbistatin.

In some embodiments of any of the methods disclosed herein, the hPSCs are maintained under conditions that result in one, two, three, or all of the following:

i) a cell density in the range of 0.5×10⁵ to 5×10⁵;

ii) a culture size of at least 1000 cm², 2000 cm², 3000cm², 4000 cm², or 5000 cm²;

iii) at least 10, 15, 20, 25, 30, 25, 40, 45, 50, 55, 60, 65, 70, 75, 80, 90 or 100 million cells; or

iv) viability of the cells, as measured by an assay of Example 1.

In some embodiments hPSCs produced by any of the methods disclosed herein, maintain at least 5%, 10%, 20%, 30%, 40%, 50%, 60%, 70%, 80%, 90% or more viable cells after a freeze-thawing cycle, e.g., after one or more cycles of freezing and/or thawing.

In some embodiments, any of the methods disclosed herein further comprise modifying the hPSCs by contacting the hPSCs with an exogenous or overexpressed molecule, e.g., a nucleotide encoding a target protein, e.g., a target protein described herein, under conditions that allow for expression of the target protein, thereby making a modified population of hPSCs.

In some embodiments, the exogenous or overexpressed molecule comprises a nucleic acid (e.g., RNA, e.g., mRNA, miRNA, or siRNA) or a protein. In some embodiments, the exogenous molecule does not naturally exist in the hPSCs. In some embodiments, the exogenous molecule induces differentiation of the hPSC into a differentiated cell, e.g., a differentiated cell described herein, e.g., a lineage committed cell, e.g., a cardiomyocyte. In some embodiments, the target protein is Wnt3a and the modified hPSCs express Wnt3a.

In some embodiments, the method further comprises monitoring the state of differentiation of the hPSCs by measuring the level of a marker, e.g., a biomarker, wherein the marker level is indicative of a particular differentiated cell, e.g., a lineage committed cell.

In some embodiments, the method further comprises modifying the hPSCs by contacting the hPSCs with an exogenous molecule that reduces the level, e.g., amount or expression, of an endogenous target in the hPSCs.

In some embodiments of any of the methods disclosed herein, the hPSCs are derived from cultured cells, e.g., a cell line, e.g., H1-hESC cell line. In some embodiments, the hPSCs are autologous to a subject, e.g., a subject to be treated.

In some embodiments of any of the methods disclosed herein, the providing of human pluripotent stem cells comprises obtaining stem cells from a subject.

In some embodiments, any of the methods disclosed herein further comprises freezing the hPSCs, e.g., under conditions that maintain viability of the hPSCs, e.g., at least 5%, 10%, 20%, 30%, 40%, 50%, 60%, 70%, 80%, 90% or more viable cells.

In some embodiments, any of the methods disclosed herein further comprises storing the hPSCs under conditions suitable for transport, e.g., to a recipient entity, e.g., a laboratory, a hospital, a health care provider.

In some embodiments, any of the methods disclosed herein further comprises thawing and preparing the hPSCs for administration into the subject.

In an aspect, provided herein is a human pluripotent stem cells (hPSC) (e.g., a population of hPSCs), e.g., human embryonic stem cells (hESCs) or induced pluripotent stem cells (iPSCs) cultured by any of the methods disclosed herein.

In another aspect, provided herein is a frozen preparation of hPSCs, comprising hPSCs cultured by any of the methods disclosed herein.

### Methods of Treating a Condition

The disclosure also provides, in an aspect, a method of treating a condition, e.g., a condition described herein (e.g., a condition associated with expression of a target protein), in a subject, comprising:

providing hPSCs received from a provider entity, e.g., wherein said provider entity has received the hPSCs cultured according to any of the methods disclosed herein, or has cultured the hPSCs according to any of the methods disclosed herein;

administering the hPSCs to the subject,

thereby treating the condition.

In an aspect, provided herein is a method of treating a condition, e.g., a condition described herein (e.g., a condition associated with expression of a target protein), in a subject, comprising:

administering the hPSCs to the subject, wherein the hPSCs are cultured, or have been cultured, according to any of the methods disclosed herein;

thereby treating the condition.

In another aspect, the disclosure provides, a method of treating a condition, e.g., a condition described herein (e.g., a condition associated with a target protein), in a subject, e.g., a subject described herein, comprising administering the hPSCs cultured according to any of the methods disclosed herein, to the subject, thereby treating the condition.

Accordingly, in an aspect, provided herein is a composition comprising hPSCs for use in a method of treating a condition, e.g., a condition described herein (e.g., a condition associated with expression of a target protein), in a subject, wherein the method comprises administering the hPSCs cultured according to any of the methods disclosed herein, to the subject, thereby treating the condition.

In some embodiments of any of the methods of treating, or compositions disclosed herein, the condition is a cardiac condition, e.g., a heart disease, e.g., myocardial infarction.

In some embodiments of any of the methods of treating, or compositions disclosed herein, the target protein is Wnt3a and the hPSCs are modified to express Wnt3a.

In some embodiments of any of the methods of treating disclosed herein, the hPSCs are administered in one or more, e.g., two, three, four or more, administrations to the subject. In some embodiments, the hPSCs are administered in repeated administrations over a specified period of time, e.g., as described herein. In some embodiments, the hPSCs are administered by intravenous, intramuscular administration, or by implantation.

Method of Reducing DID and Compositions of Matter In an aspect, provided herein is a method of reducing, e.g., inhibiting, dissociation-induced death (DID) in a population of hPSCs, comprising contacting the population of hPSCs, with an inhibitor of DID, e.g., an inhibitor of a DID activator, e.g., one or more inhibitors of a DID activator disclosed in Table 7; e.g., a PAWR inhibitor (e.g., a PAWR inhibitor described herein).

In some embodiments, the DID inhibitor, e.g., PAWR inhibitor, e.g., a PAWR inhibitor described herein results in one, two, three, four, five, or all (e.g., six) of the following:

i) a reduction in DID by at least 10%, 20%, 30%, 40%, 50%, 60%, 70%, 80%, 90%, 95%, 99%, or 100%, e.g., compared to the hPSCs in the absence of the PAWR inhibitor;

ii) a reduction in membrane blebbing as measured by an assay of Example 1 by at least 10%, 20%, 30%, 40%, 50%, 60%, 70%, 80%, 90%, 95%, 99%, or 100%, e.g., compared to the hPSCs in the absence of the PAWR inhibitor;

iii) an increase in one or more of survival, proliferation, expansion of the hPSCs by at least 10%, 20%, 30%, 40%, 50%, 60%, 70%, 80%, 90%, 95%, 99%, or 100%, e.g., compared to the hPSCs in the absence of the PAWR inhibitor;

iv) an increase in the ability to passage the hPSCs for a first, second, third, or more passages;

v) an increase in one or more of: survival, proliferation, or expansion of the hPSCs after one or more passages by at 10%, 20%, 30%, 40%, 50%, 60%, 70%, 80%, 90%, 95%, 99%, or 100%, e.g., compared to the hPSCs in the absence of the PAWR inhibitor; or

vi) an increase in the survival of hPSCs as single cells by at least 10%, 20%, 30%, 40%, 50%, 60%, 70%, 80%, 90%, 95%, 99%, or 100%, e.g., compared to the hPSCs in the absence of the PAWR inhibitor.

In some embodiments, the hPSC is cultured, e.g., manufactured, using any of the culturing methods described herein.

In some embodiments, the method further comprises contacting the population of cells with a ROCK inhibitor, e.g., Y-27632 or Thiazovivin.

In some embodiments, the method further comprises contacting the population of cells with a Myosin inhibitor, e.g., blebbistatin.

In some embodiments, the level of DID is measured by an assay of Example 1. In some embodiments, the level of DID is reduced, e.g., inhibited, compared to a population of cells cultured without the inhibitor of DID, e.g., a PAWR inhibitor.

In some embodiments, the methods disclosed herein further comprise contacting the population of cells with an additional, e.g., a second or third, DID inhibitor, e.g., an inhibitor of a DID activator listed in Table3; a Rho-dependent protein kinase (ROCK) inhibitor, e.g., a ROCK inhibitor described herein. e.g., a ROCK1 inhibitor; or a ROCK2 inhibitor, or any combination thereof. In some embodiments, the ROCK inhibitor comprises Y-27632 or Thiazovivin.

In an aspect, disclosed herein is a method of modifying a human pluripotent stem cell (hPSC), e.g., a population of hPSCs; comprising contacting the hPSCs with a DID inhibitor, e.g., an inhibitor of a DID activator, e.g., one or more inhibitors of a DID activator disclosed in Table 7; e.g., a PAWR inhibitor, (e.g., a PAWR inhibitor described herein) and an additional agent that modifies the hPSCs.

In some embodiments, the additional agent comprises an exogenous or overexpressed molecule, e.g., a nucleotide encoding a target protein, e.g., a target protein described herein, under conditions that allow for expression of the target protein. In some embodiments, the exogenous or overexpressed molecule comprises a nucleic acid (e.g., RNA, e.g., mRNA, miRNA, or siRNA) or a protein. In some embodiments, the exogenous molecule does not naturally exist in the hPSCs. In some embodiments, the exogenous molecule induces differentiation of the hPSC into a differentiated cell, e.g., a differentiated cell described herein, e.g., a lineage committed cell, e.g., a cardiomyocyte. In some embodiments, the exogenous or overexpressed molecule comprises the target protein, e.g., Wnt3a. In some embodiments, the modified hPSCs contacted with the additional agent comprising a target protein, e.g.,Wnt3a, are cultured in conditions that allow for expression of the target protein, e.g., Wnt3a.

In some embodiments, a method of modifying a hPSC disclosed herein, further comprises monitoring the state of differentiation of the hPSCs by measuring the level of a marker, e.g., a biomarker, wherein the marker level is indicative of a particular differentiated cell, e.g., a lineage committed cell.

In some embodiments, the additional agent comprises an exogenous molecule that modifies the hPSCs by reducing the level, e.g., amount or expression, of an endogenous target in the hPSCs.

In embodiments of a method of modifying hPSCs disclosed herein, the hPSCs are derived from cultured cells, e.g., a cell line, e.g., H1-hESC cell line. In some embodiments the hPSCs are autologous to a subject, e.g., a subject to be treated. In some embodiments the hPSCs are cultured, e.g., manufactured, using a method of culturing disclosed herein.

In some embodiments, the method of modifying hPSCs further comprises freezing the hPSCs, e.g., under conditions that maintain viability of the hPSCs, e.g., at least 5%, 10%, 20%, 30%, 40%, 50%, 60%, 70%, 80%, 90% or more viable cells.

Provided herein, in an aspect, is a composition comprising:

a) a population of human pluripotent stem cells (hPSCs), e.g., human embryonic stem cells (hESCs) or induced pluripotent stem cells (iPSCs);.

b) a culture medium;

c) an hPSC regulator, e.g., one, two, three or more (e.g., all) of:
(i) an inhibitor of dissociation-induced death (DID), e.g., an inhibitor of a DID activator, e.g., one or more inhibitors of a DID activator disclosed in Table 7; e.g., a PAWR inhibitor (e.g., a PAWR inhibitor described herein);
(ii) an inhibitor of stem cell pluripotency, e.g., one or more inhibitors of a target identified as OCT4 high as disclosed in Table 8;
(iii) a promoter of stem cell pluripotency, e.g., one or more activators of a target identified as OCT4 low as disclosed in Table 8;
(iv) an inhibitor of stem cell viability, e.g., cell divison, e.g., self-renewal, e.g., one or more inhibitors of a target identified as an enriched hPSC fitness gene as disclosed in Table 4; or
(v) an activator of stem cell viability, e.g., cell division, e.g., self-renewal, e.g., one or more activators of a target identified as a depleted hPSC fitness gene as disclosed in Table 4; and

d) optionally, a ROCK inhibitor, e.g., a ROCK inhibitor described herein.

In another aspect, provided herein, is a composition comprising:

a) a population of human pluripotent stem cells (hPSCs), e.g., human embryonic stem cells (hESCs) or induced pluripotent stem cells (iPSCs);

b) a culture medium;

c) a molecule to modify the hPSCs, e.g., an exogenous or overexpressed molecule;

d) an hPSC regulator, e.g., one, two, three or more (e.g., all) of:
(i) an inhibitor of dissociation-induced death (DID), e.g., an inhibitor of a DID activator, e.g., one or more inhibitors of a DID activator disclosed in Table 7; e.g., a PAWR inhibitor (e.g., a PAWR inhibitor described herein);
(ii) an inhibitor of stem cell pluripotency, e.g., one or more inhibitors of a target identified as OCT4 high as disclosed in Table 8;
(iii) a promoter of stem cell pluripotency, e.g., one or more activators of a target identified as OCT4 low as disclosed in Table 8;
(iv) an inhibitor of stem cell viability, e.g., cell divison, e.g., self-renewal, e.g., one or more inhibitors of a target identified as an enriched hPSC fitness gene as disclosed in Table 4; or
(v) an activator of stem cell viability, e.g., cell division, e.g., self-renewal, e.g., one or more activators of a target identified as a depleted hPSC fitness gene as disclosed in Table 4; and

e) optionally, an additional dissociation-induced death inhibitor, e.g., a ROCK inhibitor, e.g., a ROCK inhibitor described herein.

In some embodiments, the DID inhibitor comprises one or more of a PAWR inhibitor, or an inhibitor of a DID mediator disclosed in Table 7.

In some embodiments, the DID inhibitor comprises an inhibitor of a DID activator disclosed in Table 7. In some embodiments, the DID inhibitor is chosen from: a low molecular weight compound; an antibody molecule; an RNAi targeting (e.g., siRNA or shRNA); an epigenetic modulator of; or a genetic modulator (e.g., a nuclease, e.g., a CRISPR/Cas9, a zinc-finger nuclease (ZFN), or a Transcription activator-like effector nuclease (TALEN)).

In some embodiments, the DID inhibitor comprises a PAWR inhibitor, e.g., as described herein. In some embodiments, the PAWR inhibitor is chosen from: a low molecular weight compound inhibitor of PAWR; an anti-PAWR antibody molecule; an RNAi targeting PAWR (e.g., siRNA or shRNA); an epigenetic modulator of PAWR; or a genetic modulator of PAWR (e.g., a nuclease targeting PAWR, e.g., a CRISPR/Cas9, a zinc-finger nuclease (ZFN), or a Transcription activator-like effector nuclease (TALEN) targeting PAWR).

In some embodiments of compositions disclosed herein, the DID inhibitor, e.g., PAWR inhibitor, is provided at a dose that results in reduced, e.g., lesser, dissociation-induced death of hPSCs as measured by an assay of Example 1.

In some embodiments of compositions disclosed herein the culture medium comprises TeSR-E8 media, e.g., E8 media. In some embodiments, the culture medium further comprises about 50 mg/mL of G418, about 50 mg/uL of Doxcycyline, and about 1 mg/uL of Puromycin. In some embodiments, the E8 media further comprises about 10,000 U/mL of Penicillin-Streptomycin.

### Method of Selecting a Degron

In one aspect, the disclosure provides, a method of selecting a degron, e.g., a method of screening for a degron, comprising:

providing human pluripotent stem cells (hPSCs), e.g., a population of hPSCs, modified, e.g., by a method described herein, to express a fusion protein comprising a candidate degron and PAWR molecule (e.g., a full length, a fragment or a functional variant of PAWR); and

selecting the candidate degron when the fusion protein decreases dissociation-induced death (DID) of the hPSCs, as compared to a population of hPSCs not expressing the degron. In embodiments of a method of screening a degron disclosed herein, a decrease in DID in the modified hPSCs is due to degradation of PAWR by the degron, e.g., by targeting PAWR for proteasomal degradation. In some embodiments, DID is measured by an assay of Example 1.

In one aspect, the disclosure provides, a method of selecting a compound that regulates a degron, e.g., a method of screening for compounds that regulate a degron, comprising:

providing hPSCs expressing a fusion protein comprising a degron and PAWR;

treating the hPSCs with a candidate compound that regulates the degron; and

selecting the compound when treatment of the compound increases or decreases dissociation-induced death (DID) of the hPSCs, as compared to a population of hPSCs not treated with the compound.

The compound can be a stabilization compound or destabilization compound. If treatment of the compound increases DID of the hPSCs, the compound can be selected as a stabilization compound. If treatment of the compound decreases DID of the hPSCs, the compound can be selected as a destabilization compound.

In some embodiments, the population of hPSCs comprises, e.g., expresses, a tagged PAWR molecule, e.g., an endogenous PAWR molecule comprising a tag (e.g., a His-tag or a Flag tag) or an exogenous PAWR molecule comprising a tag (e.g., a His-tag or a Flag tag). In some embodiments, a tag is added to an endogenous PAWR molecule, e.g., to a genomic locus encoding the PAWR molecule, using a method described herein, e.g., homology directed repair. In some embodiments, the tagged endogenous PAWR molecule expresses a PAWR protein comprising the tag.

In some embodiments, the candidate degron is chosen from:

a furin degron (FurON) domain;

a degron derived from an FKB protein (FKBP);

a degron derived from dihydrofolate reductase (DHFR); a degron derived from an estrogen receptor (ER);

a degron derived from an Ikaros family of transcription factors (e.g., IKZF1, or IKZF3); or

a degron derived from a protein listed in Table 21 of International Application WO 2017/181119.

In some embodiments, the hPSCs expressing a fusion protein comprising a candidate degron can be cultured in the presence or absence of a stabilization compound, e.g., as described herein. In some embodiments, the modified hPSCs expressing a fusion protein comprising a candidate degron cultured in the absence of a stabilization compound, e.g., as described herein, have a decrease in DID as compared to modified hPSCs expressing a fusion protein comprising a candidate degron cultured in the presence of a stabilization compound.

In some embodiments, the hPSCs used in a method of selecting a degron disclosed herein are cultured, e.g., manufactured, by any of the methods of culturing disclosed herein. In some embodiments, the hPSCs are modified to express a fusion protein by contacting the population of hPSCs with a nucleotide encoding the fusion protein comprising the candidate degron and PAWR (e.g., a full length, a fragment or a functional variant of PAWR), under conditions that allow for expression of the fusion protein. In some embodiments, the hPSCs are modified to express a fusion protein by contacting the population of hPSCs with a nucleotide encoding a fusion protein, e.g., a plurality of nucleotides encoding distinct (e.g., non-identical) fusion proteins, e.g., a library of fusion proteins, wherein each fusion protein comprising the library of fusion proteins comprises a distinct degron (e.g., a non-identical degron) and PAWR e.g., a full length, a fragment or a functional variant of PAWR). In some embodiments, the hPSCs have been previously modified to not express endogenous PAWR, e.g., by a method described herein, e.g., CRISPR/Cas9.

In some embodiments, the fusion protein further comprises a protease cleavage site, e.g., a furin cleavage site. In some embodiments, the fusion protein further comprises a tag e.g., a unique identifier tag, e.g., a unique nucleotide tag comprising at least 3, 4, 5, 6, 7, 8, 9, 10, 15, 20, or 30 nucleotides. In some embodiments, the tag is used in the identification of a candidate degron obtained from the any of the screening methods described herein.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE DRAWINGS

FIGS. 1A-1H depict a self-renewing Dox-inducible CAS9/genome-wide sgRNA cell library which enables multiple high performance CRIPSR screens in human pluripotent stem cells. FIG. 1A shows a diagram depicting the iCas9 platform for genome-scale CRISPR screening in hPSCs. The iCas9 platform consists of a dox inducible Cas9 transgene knocked in the AAVS1 locus of H1-hESCs and lentiviral delivery of constitutively expressed sgRNAs. iCas9 hPSCs were transduced (0.5 MOI) at scale. After a week of expansion and selection for lentiCRISPRs iCas9 hPSCs were either banked or subjected to Cas9 mutagenesis for screening. FIG. 1B shows a correlation of normalized sgRNA counts reveals that freeze/thaw expanded samples (−dox) have high correlation with the plasmid library and the starting pool of infected iCas9 hPSCs at day 0 of the screen. Day 18 samples have been exposed to dox (+Cas9). FIG. 1C shows a diagram depicting three categories of genes that enrich (enhance), deplete (suppress) or remain constant during a fitness screen. FIG. 1D shows scatter plots depicting gene level results for core essential (dark gray) and non-essential genes (light gray). Without Cas9 treatment (−dox), cells expressing sgRNAs targeting core essential and non-essential genes are interspersed and have an RSA>−2.75 (marked by dashed line). After 18 days of exposure to Cas9 (+dox) sgRNAs targeting essential genes dropout to less than RSA −2.75. Y-axis is RSA value. X-axis marks the Z-score (Q1). Non-essential and core essential gene list from Hart et. al., 2014. FIG. 1E shows precision v. recall analysis of genome-scale CRISPR screening data in H1-hESCs. Cas9 expressing cells exhibit a PR curve that gradually slopes off, whereas cells without Cas9 exhibit a PR curve that immediately drops off. FIG. 1F shows fitness gene calculation based on 5% FDR on y-axis. Each condition labeled on the x-axis. FIG. 1G shows Venn diagram comparing 770 depleted genes in hPSCs to 1580 core essential genes identified by screening cancer cell lines (Hart et. al., 2015). 405 of the hPSCs depleted genes overlap while the remaining 365 are specifically depleted in pluripotent stem cells. FIG. 1H shows genes that dropout in the CRISPR screen are abundantly expressed. Y-axis depicts the log² transformation of average TPM values from 20 independent RNA-seq experiments in H1-hESC. For each box blot the median is depicted by white line flanked by a rectangle spanning Q1-Q3. X-axis depicts gene categories. In the first boxplot are non-essential genes from Hart et al., 2014, in the second boxplot are 405 core-essential genes, in the third boxplot are 365 stem cell-specific essential genes and in the fourth boxplot are the remaining unannotated genes.

FIGS. 2A-2C depict TP53 pathway mutations enriched during CRISPR screen in hPSCs. FIG. 2A shows a scatter plot depicting gene level results for genome-scale screen. After 18 days of exposure to Cas9 (+dox) sgRNAs targeting TP53 related genes enrich during the screen; PMAIP1 (NOXA) (RSA −10.6, Q3 3.7), TP53 (RSA −7.64, Q3 4), and CHEK2 (RSA −3.6, Q3 2.4). A total of 302 genes have a RSA score<−3 (marked by dashed line). TP53 related genes with RSA scores<−2.25 are marked in dark gray. Y-axis is a p-value generated from RSA Up analysis. X-axis marks the Z-score (Q3). FIG. 2B shows the time-dependent increase in 5 independent sgRNAs targeting PMAIP1, CHEK2 and TP53 during CRISPR screen. NGS quantified representation of lentiCRISPRs infected cells. Samples were normalized to the day 0 population and y-axis represents log 2 (fold change). Day 0 data shown is from freeze/thaw samples. X-axis plots each condition over time. Cas9+ samples were treated with dox to induce Cas9 expression. FIG. 2C shows gene knockouts (20) that enriched during CRISPR screen that are connected to TP53 and play roles in either DNA damage response and apoptosis. 952 enriched genes RSA up<−2.25 identified by STRING-DB analysis.

FIGS. 3A-3E show that PMAIP1 confers sensitivity to DNA damage in hPSCs. FIG. 3A shows a graph depicting that PMAIP1 is highly expressed in hESCs and iPSCs. Y-axis represents expression in Transcripts Per Kilobase Million (TPM). H1-hESCs (n=1), H9-hESCs (n=3), 8402-iPSCs (n=3) and HDFn-iPSCs (n=4). X-axis represents days after induction of a doxycycline inducible NGN2 expression cassette. FIG. 3B shows qPCR confirming PMAIP1 mRNA is dependent on the pluripotent state. Y-axis is relative expression and each bar represents mean relative expression. X-axis is each condition. Control=hPSCs in E8 media, +FBS=3 days exposure to 10% FBS and DMEM, +NGN2=3 days exposure to NGN2, OCT4 KO=mutant pool after 6 days exposure to iCas9 and sgRNA targeting OCT4, PMAIP1−/−=complete knockout cell line, TP53−/−=complete knockout cell line. n=3 independent mRNA samples per sgRNA, error bars+/−1 std. dev. Unpaired two-tailed t test, equal variances *p<0.05, **p<0.01. FIG. 3C shows PMAIP1 targeting sgRNAs specifically enrich during CRISPR screen in hESC but not cancer cell lines. X-axis plots CRISPR screens conducted in H1-hESC lines and 14 additional transformed lines. 5-independent sgRNAs marked by dots. Y-axis represents log 2(fold change). FIG. 3D shows PMAIP1 mutant hPSCs are insensitive to DNA damage. Live imaging of confluence in MAPT sgRNA expressing iCas9 cells+/−DSB (+dox/Cas9) in control or PMAIP1^{-/-} knockout cell line. Unlike DSB treated control cells the PMAIP1^{-/-} mutants survive in the presence of DSBs. Solid lines are without dox and dashed lines are cultured with dox. Y-axis is percent confluency each point represents mean (4 images per well, n=3 wells). Error bars+/−1 std. X-axis time in days of treatment. FIG. 3E shows qPCR of TP53 target genes indicating that PMAIP1 functions downstream of TP53. P21 and FAS mRNA is induced by MAPT targeting sgRNAs in iCas9 control cells 2 days after dox treatment. PMAIP1^{-/-} mutants exhibit increased levels of P21 and FAS mRNA which is absent in TP53^{-/-} mutants. Y-axis is relative expression is calculated by comparing the MAPT targeting sgRNA plus (+dox) or minus Cas9 expression (−dox). Unpaired two-tailed t test, equal variances *p<0.05, **p<0.01, ***p<0.001, ****p<0.0001.

FIGS. 4A-4D depict a genetic screen for suppressors of dissociation-induced death. FIG. 4A shows a diagram depicting a screen showing the dissociation and replating of the genome-scale mutant cell library without the thiazovivin (ROCK inhibitor). Most cells did not survive the treatment, however, at the end of two weeks some large colonies were recovered for DNA isolation and NGS analysis. FIG. 4B shows the screen recovered 3 out of 6 subunits of the hexameric MYOSIN motor protein that regulates blebbing in hPSCs. Y-axis average TPM in H1-hESC. X-axis myosin genes expressed >1 TPM in H1-hESCs. Myosin genes recovered by screen in light gray. Genes that were not detected by screen in dark gray. FIG. 4C shows string-DB analysis highlighting ACTIN and MYOSIN gene network among mutations that allow cells to survive dissociation in the absence of ROCK inhibitors. All genes accept ROCK2 and RAC1 were identified in the screen. FIG. 4D shows MYL6 and PAWR specifically regulate survival after dissociation and do not enrich during CRISPR screen. Each dot represents 5 independent sgRNAs per gene and NGS quantifies representation of lentiCRISPRs infected cells. Samples were normalized to the day 0 population and y-axis represents log 2(fold change). Day 0 data shown is from freeze/thaw samples maintained at 2100×. X-axis plots each condition over time. Cas9+ samples were treated with dox to induce Cas9 expression.

FIGS. 5A-5C depict that PAWR is required for dissociation-induced death. FIG. 5A shows that PAWR mutants survive single cell dissociation in the absence of thiazovivin (ROCK inhibitor) treatment. Control cells and PMAIP1 knockout do not survive without thiazovivin treatment. Bright-field images taken of live iCas9 cells 4 days after dissociation. Scale bar=800 uM. FIG. 5B shows quantification of survival in the presence or absence of thiazovivin. Percent confluence was measured 4 days after replate in control, PAWR knockout and PMAIP1 knockout cells. Bars represent mean from 3 independent wells with 4 images per well. Error bars+/−1 std. dev from 4 images per well from 3 independent wells. The dissociation induced survival of PAWR mutant hPSCs has been replicated >3 times. FIG. 5C shows results of time lapse microscopy of live cells during first 9 hours of replate. Control and PAWR knockout hPSCs survive replating in the presence of thiazovivin by extending cellular projections and forming an actin adhesion belt organized with stress fibers. Phallodin stain at 3.5 h in fixed cells. Control cells without thiazovivin have abundant membrane blebbing and this is highlighted by the presence of small circular actin rings in phallodin stained cells. PAWR mutants have reduced blebbing and an intermediate phallodin staining without small actin rings. Scale bar=50 uM.

FIGS. 6A-6C show an OCT4 FACS-based screen identifying pluripotency gene networks. FIG. 6A shows a diagram depicting FACS-based CRISPR screen using an OCT4 specific antibody to sort OCT4 high and low expressing cells. Cells were mutagenized with Cas9 for 8 days prior to FACS sorting, DNA isolation and NGS was used to identify an enrichment of sgRNAs in the high and low OCT4 populations. FIG. 6B shows scatter plots depicting gene level results for genome-scale OCT4 FACS screen. The left plot depicts OCT4^{LOW} and right plot depicts OCT4^{HIGH} enriched sgRNAs. Y-axis is a p-value generated from RSA down/up analysis. X-axis marks the Z-score (Q1/Q3). FIG. 6C shows string-DB analysis identifying a 20-gene network connected to OCT4 among gene sgRNAs that were enrich in cells with low OCT4 protein.

FIGS. 7A-7D depicts the identification of hPSC-specific essential gene networks. FIG. 7A a heatmap of Pearson correlation coefficients adapted from Hart et al., 2015 to include CRISPR screening data in H1-hESCs. FIG. 7B shows PANTHER pathway analysis identified 92 enriched pathways in hPSCs. A subset of 15 hPSC-specific pathways are depicted. FIG. 7C shows depiction of gene ontology categories including biological processes, molecular functions and developmental processes that are specific to hPSCs but not cancer cell lines. FIG. 7D shows a schematic of genes identified by CRISPR screening in hPSCs and their putative functions. 770 fitness genes regulate the self-renewing potential of hPSCs. 113 genes with low OCT4 protein are implicated in pluripotency, 99 genes with increased OCT4 protein may promote differentiation. 20 genes are implicated in the toxic response to DNA damage. 76 genes are implicated in the sensitivity of hPSCs to single cell dissociation.

FIGS. 8A-8B depict enrichment of sgRNAs on X and Y chromosomes. FIG. 8A shows a bar chart depicting the chromosomal distribution of the top 770 depleted (Table 3) and 950 enriched (RSA-up<−2.25, Table 6). FIG. 8B shows NGS quantification of indels induced by sgRNAs targeting the X chromosome. Control reads are represented by white bars, in-frame mutations by light gray bars and frameshift mutations by dark gray bars. n=1 DNA sample per sgRNA and cell line. >20,000 sequencing reads per sample.

FIGS. 9A-9F depict that PAWR is required for dissociation-induced death. FIG. 9A shows images of iCas9 expressing H1-hESCs infected with lentiCRISPRs targeting PAWR. PAWR mutant pools were created by exposing cells to Cas9/dox for one week. After mutagenesis, control and mutant cells were dissociated using accutase and replated plus or minus thiazovivin. Control cells die without thiazovivin while PAWR mutants survive. Images were taken 4 days after dissociation. FIG. 9B shows images of H1-hESC constitutively expressing dCas9-KRAB infected with CRISPRi sgRNAs targeting the promoter of PAWR. 3 out of 4 sgRNAs (i1, i2, i3, i5) tested were able to survive a replate in the absence of thiazovivin while controls died. Images were taken 6 days after dissociation. FIG. 9C shows a schematic of iCas9 H1-hESCs treated with Cas9 RNPs targeting PAWR. NGS analysis demonstrated that complete knockout of PAWR was achieved with a spectrum of frameshift indels disrupting PAWR. n=1 samples, 2621 sequencing reads. FIG. 9D shows results of karyotype analysis revealing that PAWR mutants retain a normal Karyotype. FIG. 9E shows a graph depicting that PAWR mutants do not suppress DNA damage-induced cell death. Live imaging of confluence in MAPT sgRNA expressing iCas9 cells+/−DSB (+dox/Cas9) in control, PMAIP1^{-/-} knockout cells, and PAWR^{-/-} knockout cells. PAWR^{-/-}, P21^{-/-} knockout and control hPSCs die upon DSB induction while PMAIP1^{-/-} mutants survive. Solid lines are without dox and dashed lines are cultured with Cas9 (+dox). Y-axis is percent confluency each point represents mean of 3 independent whole-well images. Error bars depict +/−1 standard deviation. X-axis time in days of treatment. FIG. 9F is a graph showing that PAWR is highly expressed in hESCs and iPSCs. Y-axis represents expression in Transcripts Per Kilobase Million (TPM). H1-hESCs (n=1), H9-hESCs (n=3), 8402-iPSCs (n=3) and HDFn-iPSCs (n=4). X-axis represents days after induction of a doxycycline inducible NGN2 expression cassette.

FIGS. 10A-10C show that PAWR is induced after dissociation and required for caspase activation. FIG. 10A shows the results of a representative immunofluorescence experiment using PAWR antibodies detects protein after dissociation but not in confluent colonies. Scale bar=100 uM. FIG. 10B shows results of a representative Immunofluorescence experiment using PAWR antibodies detecting protein after dissociation in controls but not PAWR knockout cells. FIG. 10C shows images of Western blots with control and PAWR KO cells. In control cells western blot detects caspase activation 4 hours after dissociation in the absence of thiazovivin. PAWR mutants (PAWR KO) do not exhibit caspase activation. Caspase 3 was detected with anti-rabbit Cleaved Caspase-3 (CC3, 17 and 19 kDa) antibodies. Loading controls were blotted with anti-mouse GAPDH (37 kDa) antibody.

### DETAILED DESCRIPTION

Disclosed herein, inter alia, are regulators, e.g., inhibitors or promoters, of human pluripotent stem cells (hPSCs), including but not limited to:

(i) inhibitors of dissociation-induced death (DID), e.g., inhibitors of DID activator, e.g., inhibitors of a DID activator described in Table 7;

(ii) inhibitors of stem cell pluripotency (e.g., inhibitors of one or more targets identified as OCT4 high as described in Table 8);

(iii) promoters of stem cell pluripotency (e.g., activators of one or more targets identified as OCT4 low as described in Table 8);

(iv) inhibitor of hPSC viability, e.g., cell division, e.g., self-renewal (e.g., inhibitors of one or more targets identified as an enriched hPSC fitness gene as described in Table 4), or

(v) an activator of stem cell viability, e.g., cell division, e.g., self-renewal, e.g., one or more activators of a target identified as a depleted hPSC fitness gene as disclosed in Table 4;

and methods of using the same. These hPSC regulators, e.g., inhibitors or promoters were identified by a CRISPR/Cas9 genetic screen in hPSCs, as described in Example 1. In this screen, loss of a regulator, e.g., inhibitors or promoters described herein, resulted in a specific outcome, e.g., phenotype, e.g., as described herein. For example, loss, e.g., of an inhibitor of DID, e.g., PAWR (an apoptosis regulator), or PMAIP1, resulted in inhibition of DID in hPSCs.

Accordingly, provided herein are methods of:

(a) inhibiting an activator of DID as listed in (i);

(b) inhibiting an inhibitor of stem cell pluripotency as listed in (ii);

(c) activating a promoter of stem cell pluripotency as listed in (iii); or

(d) inhibiting an inhibitor of hPSC viability, e.g., cell division, e.g., self-renewal, as listed in (iv);

(e) activating an activator of hPSC viability, e.g., cell division, e.g., self-renewal, as listed in (v)

The disclosure provides methods of:

inhibiting DID in hPSCs with an inhibitor of a DID activator, e.g., one or more inhibitors of a DID activator disclosed in Table 7, e.g., a PAWR inhibitor (e.g., a PAWR inhibitor described herein), or a PMAIP1 inhibitor (e.g., a PMAIP1 inhibitor described herein);

methods of promoting stem cell pluripotency with: an inhibitor of an inhibitor of stem cell pluripotency (e.g., one or more inhibitors of targets identified as OCT4 high as listed in Table 8), or with an activator of a promoter of stem cell pluripotency (e.g., one or more activators of targets identified as OCT4 low as listed in Table 8);

methods of promoting iPSC generation with: an inhibitor of an inhibitor of iPSC generation (e.g., one or more inhibitors of targets identified as OCT4 high as listed in Table 8), or with an activator of a promoter of iPSC generation (e.g., one or more activators of targets identified as OCT4 low as listed in Table 8); or

methods of increasing hPSC viabilitiy, e.g., cell division, e.g., self renewal, with one or more activators of targets identified as a depleted hPSC fitness gene as listed in Table 4).

Also provided herein are methods of culturing, e.g., manufacturing, hPSCs, and methods of modifying hPSCs comprising contacting the hPSCs with a regulator, e.g., inhibitor of DID (e.g., a DID inhibitor described herein), or a promoter, e.g., a promoter of stem cell pluripotency, iPSC generation or hPSC viability, e.g., as described herein; and uses thereof.

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

PRKC apoptosis WT1 regulator (PAWR), is a pro-apoptotic regulator, e.g., as described in (Hebbar, N., et al., (2012) Journal of Cellular Physiology, 227(12), 3715-3721). PAWR is also known as PAR-4. The PAWR protein is mainly cytoplasmic in the absence of apoptotic signals. In some embodiments, PAWR protein is localized in the nucleus of, e.g., cancer cells. The term “PAWR” as used herein can refer to a polypeptide or a nucleic acid encoding a polypeptide, as indicated by the context, and can include full length, a fragment or a variant of a naturally-occurring, wild type PAWR polypeptide or nucleic acid encoding the same, e.g., a functional variant, thereof. In some embodiments, PAWR can induce apoptosis by activation of the Fas pathway and inhibition of NF-kappa-B transcriptional activity. In some other embodiments, PAWR can modulate WT1 activity, e.g., by inhibiting transcriptional activation and/or enhancing transcriptional repression. In other embodiments, PAWR can down-regulate the anti-apoptotic protein BCL2. In one embodiment, the PAWR protein is encoded by the PAWR gene (NCBI Gene ID: 5074). Exemplary PAWR sequences are available at the Uniprot database under accession number Q96IZ0.

The term “PAWR molecule” as used herein can refer to a polypeptide or a nucleic acid encoding a polypeptide, as indicated by the context. This term includes full length, a fragment or a variant of a naturally-occurring, wild type PAWR polypeptide or nucleic acid encoding the same, e.g., a functional variant, thereof. In some embodiments, the variant is a derivative, e.g., a mutant, of a wild type PAWR polypeptide or nucleic acid encoding the same.

The term “dissociation-induced death” or “DID”, as used herein, is also referred to as dissociation-induced apoptosis. “Dissociation-induced death” refers to, e.g., a phenomenon in which human pluripotent stem cells (hPSCs), e.g., human embryonic stem cells (hESCs) or induced pluripotent stem cells (iPSCs), undergo cell death, e.g., apoptosis, upon dissociation, e.g., breaking of cell colonies comprising hPSCs. In some embodiments, dissociation of hPSCs into single cells can, e.g., activate Rho and Rho-dependent protein kinase (ROCK), resulting in activation of myosin. In some embodiments, activation of myosin can cause DID. In some embodiments, DID can be inhibited by, e.g., inhibiting myosin activation. In other embodiments, DID can be inhibited with a PAWR inhibitor (e.g., a PAWR inhibitor described herein); a ROCK inhibitor (e.g., a ROCK inhibitor described herein); or a myosin inhibitor (e.g., a myosin inhibitor described herein). Inhibition of DID is beneficial for culturing hPSCs, and maintaining the viability of hPSCs.

The term “inhibition” or “inhibitor” includes a reduction in a certain parameter, e.g., an activity, of a given molecule, e.g., PAWR. For example, inhibition of an activity, e.g., an activity of PAWR, of at least 5%, 10%, 20%, 30%, 40%, or more is included by this term. Thus, inhibition need not be 100%. Activities for the inhibitors can be determined as described herein or by assays known in the art. A “PAWR inhibitor” is a molecule, e.g., low molecular weight compound, antibody, RNAi agent, epigenetic modulator or genetic modulator (e.g., a nuclease, e.g., a CRISPR/Cas9, a zinc-finger nuclease (ZFN), or a Transcription activator-like effector nuclease (TALEN)), which causes the reduction in a certain parameter, e.g., an activity, e.g., dissociation-induced death of a hPSC, or which causes a reduction in a certain parameter, e.g., an activity, of a molecule associated with PAWR.

The term “cell” as used herein refers to a structural unit of a tissue of an organism, e.g., a multicellular organism. The cell can be surrounded by a membrane structure which isolates it from the outside, can have the capability of self replicating, and can have genetic information and a mechanism for expressing it. Cells may be naturally-occurring cells or artificially modified cells (e.g., fusion cells, genetically modified cells, etc.).

As used herein, the term “human pluripotent stem cells” or “hPSCs” refers to a human stem cell capable of self replication and/or pluripotency. hPSCs, as used herein, comprise but are not limited to, human embryonic stem cells (hESCs), induced pluripotent stem cells (iPSCs), tissue stem cells (e.g., tissue-specific stem cells) or somatic stem cells. Typically, hPSCs can regenerate an injured tissue. In some embodiments, hPSCs can be used to treat a disease or condition, e.g., in regenerative medicine, or personalized medicine.

The term “human embryonic stem cells” or “hESC” as used herein refers to pluripotent stem cells derived from the inner cell masse (ICM) of embryos.

The term “induced pluripotent stem cells” or “iPSCs” as used herein referred to a type of non-naturally occurring pluripotent stem cell which is artificially prepared from a non-pluripotent cell, typically an adult somatic cell, or terminally differentiated cell, such as fibroblast, a hematopoietic cell, a myocyte, a neuron, an epidermal cell, or the like, by inserting certain genes, referred to as reprogramming factors, e.g., transcription factors such as Oct4, Sox2, Klf4 and c-Myc

As used herein, “pluripotency” when used in the context of a stem cell, e.g., a hPSC, refers to the stem cells': i) potential to differentiate into cells, e.g., all cells, constituting one or more tissues or organs; ii) potential to differentiate into any one, two, or all of the three germ layers: endoderm, mesoderm, or ectoderm; or iii) potential to populate any organ or tissue.

“Self-renewal” refers to a cell division wherein a cell, e.g., a stem cell, divides into cells, wherein at least one of the cells maintains an undifferentiated, pluripotent state.

“Lineage committed cell” as used herein refers to a cell that has committed to a particular lineage, and has begun differentiation or is fully differentiated. A lineage committed cell is a cell that has lost self-renewal capacity and pluripotency. A lineage committed cell, includes, but is not limited to a cell that has begun the process of differentiation, is fully differentiated or is terminally differentiated.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “subject” is intended to include living organisms (e.g., mammals, or human).

The term “therapeutic” as used herein means a treatment. A therapeutic effect is obtained by reduction, suppression, remission, or eradication of a disease state.

An “effective amount” refers to an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A “therapeutically effective amount” of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

“Activity” of a protein refers to a function, e.g., a regulatory or biochemical function, of a protein in its native or non-native cell or tissue. Examples of activity of a protein include both direct activities and indirect activities.

The term “antibody,” as used herein, refers to a protein, or polypeptide sequence derived from an immunoglobulin molecule that specifically binds to an antigen. Antibodies can be polyclonal or monoclonal, multiple or single chain, or intact immunoglobulins, and may be derived from natural sources or from recombinant sources. Antibodies can be tetramers of immunoglobulin molecules. The term “antibody,” as used herein, also includes antibody fragments. The term “antibody fragment” refers to at least one portion of an antibody, that retains the ability to specifically interact with (e.g., by binding, steric hinderance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide brudge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3)(see U.S. Pat. No. 6,703,199, which describes fibronectin polypeptide minibodies).

The compositions and methods disclosed herein encompass polypeptides, e.g., PAWR polypeptides, and nucleic acids having the sequences specified, or sequences substantially identical or similar thereto, e.g., sequences at least 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 85%, 90%. 91%, 92%, 93%, 94%, 95%, 96%, 97%, 98% or 99% identity to a reference sequence, e.g., a sequence provided herein.

In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 85%, 90%, 91%, 92%, 93%, 94%, 95%, 96%, 97%, 98% or 99% identity to a reference sequence, e.g., a sequence provided herein.

The term “functional variant” refers to polypeptides that have a substantially identical amino acid sequence to the naturally-occurring sequence, or are encoded by a substantially identical nucleotide sequence, and are capable of having one or more activities of the naturally-occurring sequence.

The term “variant” refers to polypeptides that have a substantially identical amino acid sequence to the naturally-occurring sequence, or are encoded by a substantially identical nucleotide sequence. In some embodiments, the variant is a functional variant.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “RNAi agent” refer to an siRNA (short inhibitory RNA), shRNA (short or small hairpin RNA), iRNA (interference RNA) agent, RNAi (RNA interference) agent, dsRNA (double-stranded RNA), microRNA, and the like, which specifically binds to a target gene, and which mediates the targeted cleavage of another RNA transcript via an RNA-induced silencing complex (RISC) pathway.

The term “antisense oligonucleotide” refers to a single-stranded nucleic acid molecule having a nucleobase sequence that permits hybridization to a corresponding segment of a target nucleic acid.

The term “low molecular weight compound” is used to describe an organic or biological compound with a molecular weight of less than or equal to 2000 Da.

The term “gene editing vector” as used herein refers to a nucleic acid molecule that comprises a targeting element and/or an editing element. The target element is capable of recognizing a target genomic sequence. The editing element is capable of modifying the target genomic sequence, e.g., by subsitution or insertion of one or more nucleotides in the genomic sequence, deletion of one or more nucleotides in the genomic sequence, alteration of genomic sequences to include regulatory sequences, insertion of transgenes at a safe harbor genomic site or other specific location in the genome, or any combination thereof. The targeting element and the editing element can be on the same nucleic acid molecule or different nucleic acid molecules. The gene editing vector can be a DNA vector, an RNA vector, a plasmid, a cosmid, or a viral vector.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “autologous” refers to any material derived from the same individual to whom it is later to be re-introduced into, e.g., the same individual the material is derived from.

The term “treating” as used herein refers to the treatment of a disease or condition, e.g., a disease or condition described herein; or the prevention of a disease or condition, e.g., a disease or condition described herein; or the attenuation of one or more symptoms associated with a disease or condition, e.g., a disease or condition described herein.

As used herein, unless otherwise specified, the terms “prevent,” “preventing” and “prevention” refer to an action that occurs before the subject begins to suffer from the condition, or relapse of the condition. Prevention need not result in a complete prevention of the condition; partial prevention or reduction of the condition or a symptom of the condition, or reduction of the risk of developing the condition, is encompassed by this term.

The phrase “condition associated with expression of a target protein”, includes, but is not limited to, a disease associated with expression of a target protein described herein; or a condition associated with cells which express, or at any time expressed the target protein. Exemplary diseases or conditions include, but are not limited to neurodegenerative diseases (e.g., Parkinson's disease, amyotrophic lateral sclerosis, Alzheimer's disease, or multiple sclerosis), brain and spinal cord injury, cardiac conditions (e.g., myocardial infarction), conditions associated with abnormal hematopoietic cell formation, wound healing, teeth regeneration, hair regeneration, blindness and vision impairment, metabolic disorders (e.g., pancreatic beta cell regeneration in e.g., diabetes, e.g., juvenile-onset diabetes mellitus), and proliferative disorders, e.g., cancers.

As used herein, the term “degradation domain” or “degron” refers to a domain of a fusion polypeptide that assumes a stable conformation when expressed in the presence of a stabilization compound. Absent the stable conformation when expressed in a cell of interest, a large fraction of degradation domains (and, typically, any protein to which they are fused to, e.g., a PAWR molecule) can be degraded by endogenous cellular machinery. Thus, a degradation domain is identifiable by the following characteristics: (1) its expression is regulated co-translationally or post-translationally through increased or decreased degradation rates; and (2) the rate of degradation is substantially decreased in the presence of a stabilization compound. In some embodiments, absent a stabilization compound, the degradation domain or other domain of the fusion polypeptide is not substantially detectable in or on the cell. In some embodiments, the degradation domain is in a destabilized state in the absence of a stabilization compound. In some embodiments, the degradation domain is fused to a heterologous protease cleavage site, wherein in the presence of the stabilization compound, the cleavage of the heterologous protease cleavage site is more efficient than in the absence of the stabilization compound. In some embodiments, the degradation domain does not self-associate, e.g., does not homodimerize, in the absence of a stabilization compound. The degradation domain, e.g., degron, is not an aggregation domain as defined in WO 2017/181119 (PCT Application Number PCT/US2017/027778).

By “stabilization compound” or “stabilizing compound” is meant a compound that, when added to a cell expressing a degradation domain, stabilizes the degradation domain and decreases the rate at which it is subsequently degraded. Stabilization compounds or stabilizing compounds can be naturally occurring or synthetic.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

Human Pluripotent Stem Cells (hPSCs)

The disclosure provides, inter alia, methods of culturing, e.g., manufacturing, human pluripotent stem cells (hPSCs) and methods of modifying hPSCs, comprising contacting the cells with dissociation-induced death inhibitors. hPSCs disclosed herein include, but are not limited to human embryonic stem cells (hESCs), induced pluripotent stem cells (iPSCs), tissue stem cells (e.g., tissue-specific stem cells) or somatic stem cells.

During early embryogenesis, the inner cell mass (ICM) develops into the epiblast by becoming a polarized epithelium. Human pluripotent stem cells (hPSCs) are maintained in a primed epiblast-like state while mouse embryonic stem cells (mESCs) are maintained in a naïve ICM-like state. These developmental differences have functional consequences for the in vitro culture of human and mouse pluripotent stem cells. Unlike naïve mESCs, hPSCs are primed and are organized in a polarized epithelium which causes them, e.g., to undergo cell death during single cell dissociation. Dissociation-induced death (DID) has been a barrier to the maintenance and genetic manipulation of hPSC because it, inter alia, impedes scalability and single cell cloning.

Stem Cells and Embryonic Stem Cells (hESCs)

Stem cells are cells found in, e.g., most multi-cellular organisms. They are characterized, e.g., by the ability to self-renew, e.g., go through numerous cycles of cell division while maintaining the undifferentiated state, and maintain pluripotency, e.g., have the potential to differentiate into all cells constituting one or more tissues or organs; or, any one, two, or all of the three germ layers: endoderm, mesoderm, or ectoderm. Stem cells as disclosed herein, include but are not limited to: embryonic stem cells (hESCs) which are found in blastocysts; induced pluripotent stem cells (iPSCs) which are generated by reprogramming differentiated cells, e.g., as described herein; tissue stem cells (e.g., tissue-specific stem cells); or somatic stem cells (e.g., adult stem cells).

In a developing embryo, hESCs can differentiate into all cell types constituting one or more tissues or organs; or, any one, two, or all of the three germ layers: endoderm, mesoderm, or ectoderm. In the developed organism, e.g., post-embryonic stage, somatic stem cells and tissue stem cells can act, e.g., to replenish specialized (e.g., differentiated) cells, and also maintain the normal turnover of regenerative organs, e.g., blood, skin or intestinal tissues.

Since stem cells can be grown and differentiated into specialized cells with characteristics consistent with cells of various tissues, e.g., muscles or nerves, through cell culture, the use of these cells for therapy of conditions described herein, e.g., for regenerative medicine or tissue replacement after injury or disease, is promising. In some embodiments, cell lines derived from embryonic stem cells generated by cloning, and stem cells from, e.g., umbilical cord blood or bone marrow, can be used in the development of therapy for conditions described herein, e.g., for regenerative medicine or tissue replacement after injury or disease. In some embodiments, differentiated cells, e.g., somatic cells, can be reprogrammed with reprogramming factors (e.g., transcription factors, e.g., Oct4, Sox2, Klf4 and c-Myc) to generate induced pluripotent stem cells (iPSC) which can be used, e.g., as an alternate source of stem cells, for the development of therapy for conditions described herein, e.g., for regenerative medicine or tissue replacement after injury or disease.

Human embryonic stem cell lines (hES cell lines), e.g., the h1-hESC cell line, are cultures of human embryonic stem cells (hESCs) derived from, e.g., the epiblast tissue of the inner cell mass (ICM) of, e.g., a blastocyst or earlier morula stage embryos. A blastocyst is defined as an early stage embryo, e.g., an embryo which is about four to five days old in humans. hESCs cells are pluripotent, e.g., have the potential to give rise to one, two or all of the three primary germ layers: ectoderm, endoderm and mesoderm. In some embodiments, hESCs can develop into each of the more than, e.g., 200 cell types of the adult body when provided with, e.g., stimulation for a specific cell type.

Currently, most research has involved using mouse embryonic stem cells (mESCs) or human embryonic stem cells (hESCs). Both cell populations have the essential stem cell characteristics, and yet require very different environments in order to maintain an undifferentiated state. For example, mESCs can be grown on e.g., a layer of gelatin, and require the presence of Leukemia Inhibitory Factor (LIF). hESCs on the other hand, can be grown on a feeder layer of mouse embryonic fibroblasts (MEFs) and, e.g., require the presence of basic Fibroblast Growth Factor (bFGF or FGF-2). As disclosed in Chambers et al., 2003, embryonic stem cells, both mESCs and hESCs, can rapidly differentiate in the absence of optimal culture conditions or genetic manipulation.

In some embodiments, hESCs can also be defined by the presence of, e.g., transcription factors and cell surface proteins. In some embodiments, the transcription factors Oct-4, Nanog, and Sox2 form the core regulatory network which, e.g., controls the regulation of genes that lead to differentiation and maintenance of pluripotency (Boyer et al., 2005). In other embodiments, cell surface antigens used to identify hESCs include, but are not limited to: glycolipids, e.g., SSEA3 and SSEA4, and keratan sulfate antigens, e.g., Tra-1-60 and Tra-1-81.

In embodiments of any of the methods and compositions described herein, hPSCs can comprise cells derived from a hESC cell line, e.g., the H1-hESC cell line, also known as WA01, NIHhESC-10-0043, or WAe001-A, and provided by WiCell. In some embodiments, hPSCs derived from the H1-hESC cell line has all of the properties and characteristics of hPSCs as described herein.

### Isolation and Differentiation of Stem Cells

In some embodiments, hPSCs, e.g., hESCs, can be isolated, e.g., as described in Cowan et al. (N Engl. J. Med. 350:1353, 2004), U.S. Pat. No. 5,843,780, and Thomson et al. (Proc. Natl. Acad. Sci. USA 92:7844, 1995). In some embodiments, hPSCs, e.g., hESCs, can be prepared from human blastocyst cells using the techniques described by Thomson et al. (U.S. Pat. No. 6,200,806; Science 282:1 145, 1998; Curr. Top. Dev. Biol. 38:133 ff, 1998) and Reubinoff et al, (Nature Biotech. 18:399, 2000). In some embodiments, hPSCs, e.g., hESCs, can also be obtained from human pre-implantation embryos. In other embodiments, in vitro fertilized (FVF) embryos can be used, or one-cell human embryos can be expanded to the blastocyst stage (Bongso et al., Hum Reprod 4: 706, 1989).

In some embodiments, isolated hPSCs, e.g., hESCs, can be cultured, e.g., on a feeder layer or without a feeder layer. In some embodiments, after 9-15 days, hPSC colonies can be be dissociated into clumps, either by exposure to calcium and magnesium-free phosphate-buffered saline (PBS) with 1 mM EDTA, by exposure to dispase or trypsin, or by mechanical dissociation with a micropipette; and then replated in fresh medium. In other embodiments, growing hPSC colonies having undifferentiated morphology can be individually selected by micropipette, mechanically dissociated into clumps, and replated. In embodiments, hPSCs can be routinely split every 1-2 weeks, for example, by brief trypsinization, exposure to Dulbecco's PBS (containing 2 mM EDTA), exposure to type IV collagenase (about 200 U/mL; Gibco) or by selection of individual colonies by micropipette. In some embodiments, colony sizes of about 50 to 100 cells are optimal.

In some embodiments, hPSCs can be isolated from a sample, e.g., a sample from an organ or tissue, e.g., blood, skin, bone marrow, ovarian epithelium, testis, skeletal muscle, teeth, gut, liver, or brain, from a subject, e.g., a subject described herein. Exemplary methods of preparing a sample for isolating hPSCs from a subject are described in the Section titled “Sample preparation”.

In some embodiments, hPSCs, can be undifferentiated and subsequently differentiated into a target cell, e.g., a cell of a specific tissue as described herein, by, e.g., contacting the hPSC with an exogenous or overexpressed molecule. Differentiation of hPSCs into specific target cells and exemplary exogenous or overexpressed molecules are described in the Section titled “Target proteins for modifying hPSCs”.

Characteristics of hPSCs

In embodiments of any of the methods or compositions disclosed herein, human pluripotent stem cells (hPSCs) disclosed herein, e.g., hPSCs derived from the H1-hESC cell line, have, e.g., possess, one, two, three, four, five, six, seven, eight, nine, ten, or all (e.g., eleven) of the following characteristics or properties:

i) a morphology similar to hESCs, e.g., a single cell morphology similar to hESCs (e.g., a round shape, a large nucleolus and scant cytoplasm), or a colony morphology similar to hESCs (e.g., a sharp-edged, flat, and tightly-packed colony);

ii) growth properties, e.g., doubling time and mitotic activity similar to hESCs, e.g., as described herein;

iii) expression of stem cell markers, e.g., including, but not limited to, SSEA-3, SSEA-4, TRA-1-60, TRA-1-81, TRA-2-49/6E, and/or Nanog;

iv) expression of stem cell genes, e.g., genes expressed in undifferentiated hESCs, e.g., Oct-3/4, Sox2, Nanog, GDF3, REX1, FGF4, ESG1, DPPA2, DPPA4, and hTERT;

v) telomerase activity, e.g., as described herein;

vi) pluripotency, e.g., a potential of differentiating into one, two, or all (e.g., three) germ layers, and/or fully differentiated tissues similar to hESCs;

vii) neural differentiation, e.g., ability to differentiate into neurons, e.g., as described herein;

viii) cardiac differentiation, e.g., ability to differentiate into cardiomyocytes which can spontaneously beat, e.g., as described herein;

ix) teratoma formation, e.g., as described herein;

x) embryoid body formation, e.g., spontaneous formation of ball-like embryo-like structures in culture, e.g., as described herein; or

xi) blastocyst injection, e.g., as described herein for the formation of a chimeric organism.

In some embodiments, hPSCs disclosed herein, e.g., hPSCs derived from the H1-hESC cell line, can self-renew; and are mitotically active (e.g., capable of cell division).

In other embodiments, hPSCs disclosed herein, can proliferate and divide at a rate similar to, e.g., substantially similar to, e.g., at least 50%, 60%, 70%, 80%, 90%, 95%, 99% or 100% that of an hESC. In some embodiments, hPSCs disclosed herein can proliferate and divide at a rate similar to, e.g., 100% similar to, that of an hESC.

In some embodiments, hPSCs disclosed herein have telomerase activity similar to, e.g., at least 50%, 60%, 70%, 80%, 90%, 95%, 99% or 100% that of an hESC. Without wishing to be bound by theory, it is believed that in some embodiments, telomerase activity is necessary to sustain cell division unrestricted by the Hayflick limit of about 50 cell divisions. In some embodiments, hPSCs have high telomerase activity, e.g., higher than differentiated cells, to sustain self-renewal and proliferation. In some embodiments, hPSCs express hTERT which is the human telomerase reverse transcriptase necessary for the activity of the telomerase protein complex.

In some embodiments, hPSCs disclosed herein can differentiate into neurons, e.g., undergo neural differentiation. In some embodiments, neurons differentiated from hPSCs can express, e.g., βIII-tubulin, tyrosine hydroxylase, AADC, DAT, ChAT, LMX1B, or MAP2. In some embodiments, neurons differentiated from hPSCs can, e.g., expresss chatecholamine-associated enzymes, which indicate, e.g., a potential for differentiating into dopaminergic neurons. In some embodiments, neurons differentiated from hPSCs downregulate, e.g., reduce the expression of, stem cell-associated genes.

In some embodiments, hPSCs disclosed herein can differentiate into cardiomyocytes, e.g., undergo cardiac differentiation. In some embodiments, cardiomyocytes differentiated from hPSCs can, e.g., spontaneously begin beating. In some embodiments, cardiomyocytes differentiated from hPSCs can express, e.g., TnTc, MEF2C, MYL2A, MYHCβ, or NKX2.5. In some embodiments, cardiomyocytes differentiated from hPSCs downregulate, e.g., reduce the expression of, stem cell-associated genes.

In some embodiments, hPSCs disclosed herein can form teratomas when injected into mice, e.g., immunodeficient mice. Teratomas are tumors of multiple lineages with tissue or cells derived from all three germ layers. In some embodiments, teratoma formation is a measure of pluripotency.

In some embodiments, hPSCs disclosed herein can form embryoid bodies in culture. Embryoid bodies are ball-like embryo-like structures which include a core that is mitotically active comprising stem cells and a periphery comprising differentiated cells, e.g., from all three germ layers.

Culturing hPSCs

In some embodiments, hPSCs, e.g., hESCs or iPSCs, are cultured as colonies, e.g., not as single cells. Without wishing to be bound by theory, it is believed that in some embodiments, hPSCs undergo apoptosis when dissociated from the cell colonies, e.g., when cultured as single cells.

In embodiments of any of the methods and compositions disclosed herein, hPSCs can be cultured, e.g., manufactured, in culture medium comprising TeSR-E8 media (referred to as E8 media). In some embodiments, the E8 media comprises about 50 mg/mL of G418, about 50 mg/uL of Doxcycyline, and about 1 mg/uL of Puromycin. In some embodiments, the E8 media further comprises about 8 mm of Thiazovivin. In other embodiments, the E8 media further comprises about 10,000 U/mL of Penicillin-Streptomycin.

In some embodiments, culture medium used for culturing hPSCs disclosed herein can comprise a medium, e.g., basal medium, containing salts, vitamins, glucose and amino acids. The medium can be any of a number of commercially available media. In some embodiments, the medium comprises a combination of Dulbecco's Modified Eagle Medium and Hams F12 medium, sold as a combination (DMEM/F12). In some embodiments, the medium comprises glutamine, β-mercaptoethanol, and non-essential amino acids. Other possible additives include antioxidants and lipids. In some embodiments, the medium comprises a protein constituent, e.g., a serum substitute product, e.g., albumin or purified albumin products such as AlbuMax™. In some embodiments, the protein consitutent comprises albumin, insulin and transferrin.

In some embodiments, hPSCs can be cultured, e.g., manufactured, in culture medium comprising: 80% DMEM (Gibco #10829-018 or #11965-092), 20% defined fetal bovine serum (FBS) not heat inactivated, 1% non-essential amino acids, 1 mM L-glutamine, and 0.1 mM .beta.-mercaptoethanol. In some embodiments, hPSCs can be cultured in serum-free medium, made with 80% Knock-Out DMEM (Gibco #10829-018), 20% serum replacement (Gibco #10828-028), 1% non-essential amino acids, 1 mM L-glutamine, and 0.1 mM .beta.-mercaptoethanol. In some embodiments, the culture medium can further comprise human bFGF at a final concentration of .about 4 ng/mL, as described in U.S. Pat. No. 7,297,539, the entire contents of which are hereby incorporated by reference.

In some embodiments, hPSCs can be cultured, e.g., manufactured, in culture medium comprising UM100 media comprising: unconditioned media (UM) consisting of 80% (v/v) DMEM/F12 (Gibco/Invitrogen) and 20% (v/v) KNOCKOUT™ SR (Gibco/Invitrogen), about 1 mM glutamine (Gibco/Invitrogen),about 0.1 mM β-mercaptoethanol (Sigma—St. Louis, Mo.), and about 1% nonessential amino acid stock (Gibco/Invitrogen). In some embodiments, the media further comprises 100 ng/ml bFGF. In some embodiments, the medium is filtered through a 0.22 μM nylon filter (Nalgene).

In some embodiments, hPSCs can be cultured, e.g., manufactured, in culture medium comprising BM+ medium comprising: DMEM/F12 (Gibco/Invitrogen), about 16.5 mg/ml BSA (Sigma), about 196 μg/ml Insulin (Sigma), about 108 μg/ml Transferrin (Sigma), about 100 ng/ml bFGF, about 1 mM glutamine (Gibco/Invitrogen), about 0.1 mM β-mercaptoethanol (Sigma), and about 1% nonessential amino acid stock (Gibco/Invitrogen). In some embodiments, the osmolality of the medium is adjusted to 340 mOsm with 5M NaCl. In some embodiments, the medium is filtered through a 0.22 μM nylon filter (Nalgene).

In some embodiments, hPSCs can be cultured, e.g., manufactured, in culture medium comprising DHEM medium comprising: DMEM/F12 (Gibco/Invitrogen), about 16.5 mg/ml HSA (Sigma), about 196 μg/ml Insulin (Sigma), about 108 μg/ml Transferrin (Sigma), about 100 ng/ml bFGF, about 1 mM glutamine (Gibco/Invitrogen), about 0.1 mM β-mercaptoethanol (Sigma), about 1% nonessential amino acid stock (Gibco/Invitrogen), vitamin supplements (Sigma), trace minerals (Cell-gro®), and 0.014 mg/L to 0.07 mg/L selenium (Sigma).). In some embodiments, the osmolality of the medium is adjusted to 340 mOsm with 5M NaCl. In some embodiments, the vitamin supplements in the medium may include thiamine (6.6 g/L), reduced glutathione (2 mg/L) and ascorbic acid PO4. In some embodiments, the trace minerals used in the medium are a combination of Trace Elements B (Cell-gro®, Cat #: MT 99-175-Cl and C (Cell-gro®, Cat #: MT 99-176-Cl); each of which is at a 1,000× solution. In some embodiments, the medium further comprises defined lipids (Gibco/Invitrogen).

Additional culture media for culturing hPSCs are disclosed in U.S. Pat. No. 7,005,252, the entire contents of which are hereby incorporated by reference.

In some embodiments, hPSCs cultured in any of the methods disclosed herein can be assessed for the expression of antigens, e.g., antigens that are characteristic of hESCs, e.g., SSEA-1, SSEA-3, SSEA-4, TRA-1-60 and TRA-1-81. In some embodiments, expression of the antigens can be performed by immunohistochemistry or flow cytometry methods well-known in the art.

In embodiments, hPSCs cultured in any of the methods disclosed herein maintain, e.g., preserve, viability of at least 50%, 60%, 70%, 80%, 90%, 95%, 99%, or 100% of cells.

In some embodiments, hPSCs cultured in any of the methods disclosed herein does not results in loss of viable cells, e.g., less than 50%, 40%, 30%, 20%, 15%, 10%, 9%, 8%, 7%, 6%, 55, 4%, 35, 25, 15, or lesser loss in viable cells.

In embodiments of any of the methods and compositions disclosed herein, hPSCs can be cultured, e.g., manufactured, without a feeder layer.

In embodiments of any of the methods and compositions disclosed herein, hPSCs can be cultured, e.g., manufactured, in a multi-layer culture vessel, e.g., a vessel with 2, 3, 4, 5, or more layers. In some embodiments, the hPSCs can be cultured in a 2-layer vessel, e.g., a 2-layer CellSTACK vessel with a culture area of about 1272 cm². In some embodiments, the hPSCs can be cultured in a 5-layer vessel, e.g., a 5-layer CellSTACK vessel with a culture area of about 3138 cm². In some embodiments, the vessels can be coated with vitronectin.

In embodiments of any of the methods and compositions disclosed herein, hPSCs can be cultured, e.g., manufactured, for at least 3 passages, e.g., at least 3, 4, 5, 6, 7, 8, 9, 10, 11, 12, 13, 14, 15, 16, 17, 18, 19, 20, 30, 40, 50, 60, 70, 80, 90, 100 or more, passages. In embodiments, hPSCs cultured, e.g., manufactured, for at least 3 passages maintain, e.g., preserve, viability of at least 50%, 60%, 70%, 80%, 90%, 95%, 99%, or 100% of cells. In embodiments, culturing, e.g., manufacturing, for at least 3 passages does not result in loss of viable cells, e.g., less than 50%, 40%, 30%, 20%, 15%, 10%, 9%, 8%, 7%, 6%, 55, 4%, 35, 25, 15, or lesser loss in viable cells.

In embodiments, hPSCs cultured, e.g., manufactured, by any of the methods disclosed herein maintain one, two, three, four, five, six, seven, eight, nine, ten, or all (e.g., eleven) of the following characteristics or properties:

i) a morphology similar to hESCs, e.g., a single cell morphology similar to hESCs (e.g., a round shape, a large nucleolus and scant cytoplasm), or a colony morphology similar to hESCs (e.g., a sharp-edged, flat, and tightly-packed colony);

ii) growth properties, e.g., doubling time and mitotic activity similar to hESCs, e.g., as described herein;

iii) expression of stem cell markers, e.g., including, but not limited to, SSEA-3, SSEA-4, TRA-1-60, TRA-1-81, TRA-2-49/6E, and/or Nanog;

iv) expression of stem cell genes, e.g., genes expressed in undifferentiated hESCs, e.g., Oct-3/4, Sox2, Nanog, GDF3, REX1, FGF4, ESG1, DPPA2, DPPA4, and hTERT;

v) telomerase activity, e.g., as described herein;

vi) pluripotency, e.g., a potential of differentiating into one, two, or all (e.g., three) germ layers, and/or fully differentiated tissues similar to hESCs;

vii) neural differentiation, e.g., ability to differentiate into neurons, e.g., as described herein;

viii) cardiac differentiation, e.g., ability to differentiate into cardiomyocytes which can spontaneously beat, e.g., as described herein;

ix) teratoma formation, e.g., as described herein;

x) embryoid body formation, e.g., spontaneous formation of ball-like embryo-like structures in culture, e.g., as described herein; or

xi) blastocyst injection, e.g., as described herein for the formation of a chimeric organism.

In some embodiments, at least 50%, 60%, 70%, 80%, 90%, 95%, 99%, or 100% of the hPSCs cultured, e.g., manufactured, by any of the methods disclosed herein maintain properties or characteristics defined in (i)-(xi). In some embodiments, at least 50%, 60%, 70%, 80%, 90%, 95%, 99%, or 100% of the hPSCs cultured, e.g., manufactured, by any of the methods disclosed herein maintain properties or characteristics defined in (i)-(xi) for at least 3 passages, e.g., at least 3, 4, 5, 6, 7, 8, 9, 10, 11, 12, 13, 14, 15, 16, 17, 18, 19, 20, 30, 40, 50, 60, or more, passages.

In embodiments, hPSCs cultured, e.g., manufactured, by any of the methods disclosed herein remain euploid and retain stable karyotypes.

In some embodiments, hPSCs cultured in any of the methods disclosed herein have a growth rate similar to, e.g., at least 50%, 60%, 70%, 80%, 90%, 95%, 99%, or 100% similar to that of a hESC. In some embodiments, the growth rate of hPSCs, e.g., hESCs or iPSCs, can be determined by platined cells at a density of about 5×105 cells/well in triplicate in 6-well tissue culture dishes (Nalgene). On days 3, 5, and 7 the triplicate wells can be treated with trypsin/EDTA (Gibco/Invitrogen), individualized and cell numbers can be counted. On day 7, additional wells can be treated with trypsin, counted, and used to re-seed a new plate at a cell density of about 2×105 cells/well. The day 7 cultures, which have been trypsin processed, can be analyzed for hPSC cell surface markers, e.g., Oct4, SSEA4, or Tra1-60 by, e.g., flow cytometry, e.g., FACS analysis. In some embodiments, growth rates can be collected for 3 consecutive passages.

### Dissociation-Induced Death and Inhibitors Thereof

Dissociation-induced death (DID), also known as dissociation-induced apoptosis, refers to a phenomenon in which human pluripotent stem cells (hPSCs), e.g., human embryonic stem cells (hESCs) or induced pluripotent stem cells (hIPSCs), undergo apoptosis upon dissociation, e.g., breaking of the cell colonies comprising hPSCs. In some embodiments, the dissociation of hPSCs into single cells activates, e.g., Rho and Rho-dependent protein kinase (ROCK), which results in the phosphorylation, e.g., activation, of Myosin. In some embodiments, the dissociation of hPSCs into single cells results in DID which can be due to, e.g., the activation of Myosin which induces, e.g., membrane blebbing. In some embodiments, DID comprises cell death (e.g., apoptosis), membrane blebbing and/or activation of myosin (e.g., hyperactivation of the actomyosin network).

Dissociation of hPSCs into single cells is important for passaging, e.g., growing, the cells for culturing (e.g., manufacturing), expansion, manipulation or maintenance of the hPSCs. In some embodiments, DID in hPSCs can be prevented, e.g., reduced, by contacting hPSCs with an inhibitor of DID, e.g., an inhibitor described herein, thereby maintaining the viability of the hPSCs, e.g., preventing apoptosis. In some embodiments, a DID inhibitor is an inhibitor of an activator of DID. In some embodiments, DID activators, include but are not limited to targets listed in Table 7, e.g., PAWR. In some embodiments, a DID inhibitor comprises a PRKC apoptosis WT1 regulator (PAWR) inhibitor, a Myosin inhibitor, or a ROCK inhibitor, In some embodiments, inhibition of ROCK prevents dissociation-induced death mediated hyperactivation of the actomyosin network, membrane blebbing and cell death of hPSCs.

In some embodiments, DID can be prevented, e.g., reduced, by contacting the hPSCs with a DID inhibitor, e.g., an inhibitor of a DID activator listed in Table 7, e.g., a PAWR inhibitor (e.g., a PAWR inhibitor described herein). Exemplary PAWR inhibitors are described in the section titled “PAWR inhibitor”.

In some embodiments, DID can be prevented, e.g., reduced, by contacting the hPSCs with a ROCK inhibitor, e.g., Y-27632 or Thiazovivin.

In some embodiments, DID can be prevented, e.g., reduced, by contacting the hPSCs with a Myosin inhibitor, e.g., blebbistatin.

### PAWR Inhibitor

This disclosure provides, inter alia, a novel role for PAWR in, e.g., dissociation-induced death (DID) of hPSCs. In embodiments, loss, e.g., genetic loss via CRISPR/Cas9, of PAWR was shown to inhibit, e.g., reduce, DID in hPSCs cultured in the absence of ROCK or Myosin inhibitors. In some embodiments, disclosed herein is a PAWR inhibitor for use in any of the methods or compositions described herein for reducing, e.g., inhibiting, DID in hPSCs, thereby maintaining the viability of the hPSCs, e.g., preventing (e.g., reducing) apoptosis.

PAWR, also known as PRKC apoptosis WT1 regulator, is a pro-apoptotic regulator (Hebbar, N., et al., (2012) Journal of Cellular Physiology, 227(12), 3715-3721). The PAWR protein is mainly cytoplasmic in the absence of apoptotic signals. In some embodiments, PAWR protein is localized in the nucleus of cancer cells. In some embodiments, PAWR localized in the cytoplasm can, e.g., bind actin (e.g., F-actin) and/or myosin. Binding of PAWR to actin and/or myosin is disclosed, e.g., in Vetterkind S. et al., (2005) Experimental Cell Research, 35(2), 392-408, and Vetterkind, S., & Morgan, K. G. (2009) Journal of Cellular and Molecular Medicine, 13(5), 887-895.

In some embodiments, PAWR can induce apoptosis by activation of the Fas pathway and inhibition of NF-kappa-B transcriptional activity. In some other embodiments, PAWR can modulate WT1 activity, e.g., by inhibiting transcriptional activation and/or enhancing transcriptional repression. In other embodiments, PAWR can down-regulate the anti-apoptotic protein BCL2.

In some embodiments, a PAWR inhibitor can be used in any of the methods or compositions described herein for reducing, e.g., inhibiting, DID in hPSCs, thereby maintaining the viability of the hPSCs, e.g., preventing (e.g., reducing) apoptosis. In some embodiments, the PAWR inhibitor comprises an inhibitor chosen from: a small molecule inhibitor of PAWR; an anti-PAWR antibody molecule; an RNAi targeting PAWR (e.g., siRNA or shRNA); an epigenetic modulator of PAWR; or a genetic modulator of PAWR (e.g., a nuclease targeting PAWR, e.g., a CRISPR/Cas9, or a CRISPR/C2c2 (e.g., CRISPR/Cas13a), a zinc-finger nuclease

(ZFN), or a Transcription activator-like effector nuclease (TALEN) targeting PAWR). In some embodiments, the PAWR inhibitor is a low molecular weight compound inhibitor of PAWR, e.g., a low molecular weight compound inhibitor of PAWR described herein. In some embodiments, the PAWR inhibitor is an anti-PAWR antibody molecule, e.g., anti-PAWR antibody described herein. In some embodiments, the PAWR inhibitor is an RNAi targeting PAWR (e.g., siRNA or shRNA); e.g., an RNAi targeting PAWR (e.g., siRNA or shRNA) described herein. In some embodiments, the PAWR inhibitor is an epigenetic modulator of PAWR, e.g., an epigenetic modulator of PAWR described herein. In some embodiments, the PAWR inhibitor is a genetic modulator of PAWR, e.g., a nuclease targeting PAWR (e.g., a CRISPR/Cas9, or a CRISPR/C2c2 (e.g., CRISPR/Cas13a), a zinc-finger nuclease (ZFN), or a Transcription activator-like effector nuclease (TALEN) targeting PAWR). In some embodiments, the PAWR inhibitor is a CRISPR/Cas9 targeting PAWR.

### PMAIP1 Inhibitor

PMAIP1, or Phorbol-12-Myristate-13-Acetate-Induced Protein 1, is also known as NOXA. PMAIP1 or NOXA is a pro-apoptotic regulator and belongs to the Bcl-2 family. PMAIP1 has been shown to be regulated by p53 and is implicated in p53-mediated cell death, e.g., apoptosis, as described, e.g., in Oda E, et al., (2000) Science Vol. 288, Issue 5468, pp. 1053-1058. In some embodiments, PMAIP1 acts as a stem cell apoptosis sensitizer, e.g., PMAIP1 increases the sensitivity of stem cells to apoptotic triggers, e.g., double strand breaks.

Without wishing to be bound by theory, it is believed that in some embodiments, a PMAIP1 inhibitor inhibits the p53 pathway, e.g., p53. In some embodiments, a PMAIP1 inhibitor disclosed herein is a p53 inhibitor. In some embodiments, a PMAIP1 inhibitor is used to inhibit the p53 pathway (e.g., cellular responses to p53), in methods of genome engineering, e.g., as discussed herein. In some embodiments, methods of genome engineering in hPSCs comprise the use of a PMAIP1 inhibitor, e.g., to inhibit the p53 pathway, e.g., cellular responses to p53. In some embodiments, the PMAIP1 inhibitor is used with an additional p53 inhibitor. In some embodiments, the PMAIP inhibitor is the only p53 inhibitor used.

In some embodiments of any of the methods of compositions disclosed herein, PMAIP1 is expressed in hPSCs, e.g., as described in Example 1. In some embodiments, PMAIP1 expression in hPSCs is not regulated by, e.g., OCT4. In some embodiments, PMAIP1 expression in hPSCs, regulates DNA damage response in hPSCs, e.g., sensitizes hPSCs to DNA damage, e.g., as described in an assay of Example 1.

In some embodiments, a PMAIP1 inhibitor can be used in methods and/or compositions for decreasing toxicity of a gene editing system and/or increasing gene editing efficiency (e.g., as a TP53 inhibitor) as described in PCT/IB2017/056791, which is incorporated by reference herein. In some embodiments, the PMAIP1 inhibitor comprises an inhibitor chosen from: a small molecule inhibitor of PMAIP1; an anti-PMAIP1antibody molecule; an RNAi targeting PMAIP1 (e.g., siRNA or shRNA); an epigenetic modulator of PMAIP1; or a genetic modulator of PMAIP1 (e.g., a nuclease targeting PMAIP1, e.g., a CRISPR/Cas9, a zinc-finger nuclease (ZFN), or a Transcription activator-like effector nuclease (TALEN) targeting PMAIP1). In some embodiments, the PMAIP1 inhibitor is a low molecular weight compound inhibitor of PMAIP1, e.g., a low molecular weight compound inhibitor of PMAIP1 described herein. In some embodiments, the PMAIP1 inhibitor is an anti-PMAIP1 antibody molecule, e.g., anti-PMAIP1 antibody described herein. In some embodiments, the PMAIP1 inhibitor is an RNAi targeting PMAIP1 (e.g., siRNA or shRNA); e.g., an RNAi targeting PMAIP1 (e.g., siRNA or shRNA) described herein. In some embodiments, the PMAIP1 inhibitor is an epigenetic modulator of PMAIP1, e.g., an epigenetic modulator of PMAIP1 described herein. In some embodiments, the PMAIP1 inhibitor is a genetic modulator of PMAIP1, e.g., a nuclease targeting PMAIP1 (e.g., a CRISPR/Cas9, a zinc-finger nuclease (ZFN), or a Transcription activator-like effector nuclease (TALEN) targeting PMAIP1). In some embodiments, the PMAIP1 inhibitor is a CRISPR/Cas9 targeting PMAIP1.

### RNAi Agents

As used herein, the term “RNAi agent,” “RNAi agent to a DID activator disclosed in Table 7, e.g., PAWR or PMAIP1”, “siRNA to a DID activator disclosed in Table 7, e.g., PAWR or PMAIP1”, “PAWR or PMAIP1 siRNA” and the like refer to an siRNA (short inhibitory RNA), shRNA (short or small hairpin RNA), iRNA (interference RNA) agent, RNAi (RNA interference) agent, dsRNA (double-stranded RNA), microRNA, and the like, which specifically binds to a target described herein, e.g., the PAWR gene, and which mediates the targeted cleavage of another RNA transcript via an RNA-induced silencing complex (RISC) pathway. In one embodiment, the RNAi agent is an oligonucleotide composition that activates the RISC complex/pathway. In another embodiment, the RNAi agent comprises an antisense strand sequence (antisense oligonucleotide). In one embodiment, the RNAi comprises a single strand. This single-stranded RNAi agent oligonucleotide or polynucleotide can comprise the sense or antisense strand, as described by Sioud 2005 J. Mol. Biol. 348:1079-1090, and references therein. Thus the disclosure encompasses RNAi agents with a single strand comprising either the sense or antisense strand of an RNAi agent described herein. The use of the RNAi agent to a target results in a decrease of target activity, level and/or expression, e.g., a “knock-down” or “knock-out” of the target gene or target sequence.

RNA interference is a post-transcriptional, targeted gene-silencing technique that, usually, uses double-stranded RNA (dsRNA) to degrade messenger RNA (mRNA) containing the same sequence as the dsRNA. The process of RNAi occurs naturally when ribonuclease III (Dicer) cleaves longer dsRNA into shorter fragments called siRNAs. Naturally-occurring siRNAs (small interfering RNAs) are typically about 21 to 23 nucleotides long and comprise about 19 base pair duplexes. The smaller RNA segments then mediate the degradation of the target mRNA. Dicer has also been implicated in the excision of 21- and 22-nucleotide small temporal RNAs (stRNAs) from precursor RNA of conserved structure that are implicated in translational control. Hutvagner et al. 2001, Science, 293, 834. The RNAi response also features an endonuclease complex, commonly referred to as an RNA-induced silencing complex (RISC), which mediates cleavage of single-stranded mRNA complementary to the antisense strand of the siRNA. Cleavage of the target RNA takes place in the middle of the region complementary to the antisense strand of the siRNA duplex.

“RNAi” (RNA interference) has been studied in a variety of systems. Early work in Drosophila embryonic lysates (Elbashir et al. 2001 EMBO J. 20: 6877 and Tuschl et al. International PCT Publication No. WO 01/75164) revealed certain parameters for siRNA length, structure, chemical composition, and sequence that are beneficial to mediate efficient RNAi activity. These studies have shown that 21-nucleotide siRNA duplexes are most active when containing 3′-terminal dinucleotide overhangs. Substitution of the 3′-terminal siRNA overhang nucleotides with 2′-deoxy nucleotides (2′-H) was tolerated. In addition, a 5′-phosphate on the target-complementary strand of an siRNA duplex is usually required for siRNA activity. Later work showed that a 3′-terminal dinucleotide overhang can be replaced by a 3′ end cap, provided that the 3′ end cap still allows the molecule to mediate RNA interference; the 3′ end cap also reduces sensitivity of the molecule to nucleases. See, for example, U. S. Pat. Nos. 8,097,716; 8,084,600; 8,404,831; 8,404,832; and 8,344,128. Additional later work on artificial RNAi agents showed that the strand length could be shortened, or a single-stranded nick could be introduced into the sense strand. In addition, mismatches can be introduced between the sense and anti-sense strands and a variety of modifications can be used. Any of these and various other formats for RNAi agents known in the art can be used to produce RNAi agents to a target described herein, e.g., PAWR. In some embodiments, the RNAi agent to a target described herein, e.g., PAWR, is ligated to one or more diagnostic compound, reporter group, cross-linking agent, nuclease-resistance conferring moiety, natural or unusual nucleobase, lipophilic molecule, cholesterol, lipid, lectin, steroid, uvaol, hecigenin, diosgenin, terpene, triterpene, sarsasapogenin, Friedelin, epifriedelanol-derivatized lithocholic acid, vitamin, carbohydrate, dextran, pullulan, chitin, chitosan, synthetic carbohydrate, oligo lactate 15-mer, natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

Kits for RNAi synthesis are commercially available, e.g., from New England Biolabs and Ambion.

A suitable RNAi agent can be selected by any process known in the art or conceivable by one of ordinary skill in the art. For example, the selection criteria can include one or more of the following steps: initial analysis of the target gene sequence and design of RNAi agents; this design can take into consideration sequence similarity across species (human, cynomolgus, mouse, etc.) and dissimilarity to other (non-target) genes; screening of RNAi agents in vitro (e.g., at 10 nM in cells); determination of EC50 in HeLa cells; determination of viability of various cells treated with RNAi agents, wherein it is desired that the RNAi agent to a target not inhibit the viability of these cells; testing with human PBMC (peripheral blood mononuclear cells), e.g., to test levels of TNF-alpha to estimate immunogenicity, wherein immunostimulatory sequences are less desired; testing in human whole blood assay, wherein fresh human blood is treated with an RNAi agent and cytokine/chemokine levels are determined [e.g., TNF-alpha (tumor necrosis factor-alpha) and/or MCP1 (monocyte chemotactic protein 1)], wherein immunostimulatory sequences are less desired; determination of gene knockdown in vivo using subcutaneous tumors in test animals; target gene modulation analysis, e.g., using a pharmacodynamic (PD) marker, and optimization of specific modifications of the RNAi agents.

In some embodiments, the present invention provides a RNAi agent to a target described herein, e.g., PAWR, and methods of using a RNAi agent to target a target described herein, e.g., PAWR. RNAi agents disclosed herein include those compositions capable of mediating RNA interference, including, inter alia, shRNAs and siRNAs. In some embodiments, the RNAi agent comprises an antisense strand and a sense strand.

An embodiment of the invention provides a composition comprising an RNAi agent comprising a first (sense) or second (antisense) strand, wherein the sense and/or antisense strand comprises at least 15 contiguous nucleotides differing by 0, 1, 2, or 3 nucleotides from the antisense strand of an RNAi agent to a target described herein, e.g., PAWR. In another embodiment, the present invention provides a composition comprising an RNAi agent comprising a sense strand and an antisense strand, wherein the antisense strand comprises at least 15 contiguous nucleotides differing by 0, 1, 2, or 3 nucleotides from the antisense strand of an RNAi agent.

In another embodiment, the present invention provides a composition comprising an RNAi agent comprising a sense strand and an antisense strand, wherein the sense strand comprises at least 15 contiguous nucleotides differing by 0, 1, 2, or 3 nucleotides from the sense strand and the antisense strand comprises at least 15 contiguous nucleotides differing by 0, 1, 2, or 3 nucleotides from the antisense strand of an RNAi agent to a target described herein, e.g., PAWR.

In one embodiment, the present invention provides particular compositions comprising an RNAi agent comprising an antisense strand, wherein the antisense strand comprises at least 15 contiguous nucleotides from the antisense strand of an RNAi agent to a target described herein, e.g., PAWR. In another embodiment, the present invention provides a composition comprising an RNAi agent comprising a sense strand and an antisense strand, wherein the sequence of the antisense strand is the sequence of the antisense strand of an RNAi agent to a target described herein, e.g., PAWR. In another embodiment, the present invention provides a composition comprising an RNAi agent comprising a sense strand and an antisense strand, wherein the sequence of the antisense strand comprises the sequence of the antisense strand of an RNAi agent to a target described herein, e.g., PAWR.

In some embodiments, the antisense and sense strand can be two physically separated strands, or can be components of a single strand or molecule, e.g., they are linked a loop of nucleotides or other linker. A non-limiting example of the former is a siRNA; a non-limiting example of the latter is a shRNA. The can also, optionally, exist single-stranded nicks in the sense strand, or one or more mismatches between the antisense and sense strands.

Additional modified sequences (e.g., sequences comprising one or more modified base) of each of the compositions above are also contemplated as part of the disclosure.

In one embodiment, the antisense strand is about 30 or fewer nucleotides in length. In one embodiment, the antisense strand forms a duplex region with a sense strand, wherein the duplex region is about 15 to 30 nucleotide pairs in length.

In one embodiment, the antisense strand is about 15 to about 30 nucleotides in length, including about 19 to about 23 nucleotides in length. In one embodiment, the antisense strand has at least the length selected from about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides and 30 nucleotides.

In one embodiment, the RNAi agent comprises a modification that causes the RNAi agent to have increased stability in a biological sample or environment.

In one embodiment, the RNAi agent comprises at least one sugar backbone modification (e.g., phosphorothioate linkage) or at least one 2′-modified nucleotide.

In one embodiment, the RNAi agent comprises: at least one 5′-uridine-adenine-3′ (5′-ua-3′) dinucleotide, wherein the uridine is a 2′-modified nucleotide; at least one 5′-uridine-5 guanine-3′ (5′-ug-3′) dinucleotide, wherein the 5′-uridine is a 2′-modified nucleotide; at least one 5′-cytidine-adenine-3′ (5′-ca-3′) dinucleotide, wherein the 5′-cytidine is a 2′-modified nucleotide; or at least one 5′-uridine-uridine-3′ (5′-uu-3′) dinucleotide, wherein the 5′-uridine is a 2′-modified nucleotide. These dinucleotide motifs are particularly prone to serum nuclease degradation (e.g. RNase A). Chemical modification at the 2′-position of the first pyrimidine nucleotide in the motif prevents or slows down such cleavage. This modification recipe is also known under the term ‘endo light’.

In one embodiment, the RNAi agent comprises a 2′-modification selected from the group consisting of: 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), and 2′-O-N-methylacetamido (2′-O-NMA). In one embodiment, all pyrimidines (uridine and cytidine) are 2′-O-methyl-modified nucleosides. In some embodiments, one or more nucleotides can be modified, or substituted with DNA, a peptide nucleic acid (PNA), locked nucleic acid (LNA), morpholino nucleotide, threose nucleic acid (TNA), glycol nucleic acid (GNA), arabinose nucleic acid (ANA), 2′-fluoroarabinose nucleic acid (FANA), cyclohexene nucleic acid (CeNA), anhydrohexitol nucleic acid (HNA), unlocked nucleic acid (UNA).

In some embodiments, the sense and/or antisense strand can terminate at the 3′ end with a phosphate or modified internucleoside linker, and further comprise, in 5′ to 3′ order: a spacer, a second phosphate or modified internucleoside linker, and a 3′ end cap. In some embodiments, modified internucleoside linker is selected from phosphorothioate, phosphorodithioate, phosphoramidate, boranophosphonoate, an amide linker, and a compound of formula (I):

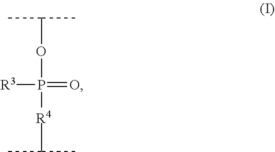

where R3 is selected from O—, S—, NH2, BH3, CH3, C1-6 alkyl, C6-10 aryl, C1-6 alkoxy and C6-10 aryl-oxy, wherein C1-6 alkyl and C6-10 aryl are unsubstituted or optionally independently substituted with 1 to 3 groups independently selected from halo, hydroxyl and NH2; and R4 is selected from O, S, NH, and CH2. In some embodiments, the spacer can be a sugar, alkyl, cycloakyl, ribitol or other type of a basic nucleotide, 2′-deoxy-ribitol, diribitol, 2′-methoxyethoxy-ribitol (ribitol with 2′-MOE), C3-6 alkyl, or 4-methoxybutane-1,3-diol (5300). In some embodiments, the 3′ end cap can be selected from any of various 3′ end caps described herein or known in the art. In some embodiments, one or more phosphates can be replaced by a modified internucleoside linker.

In one embodiment, the RNAi agent comprises at least one blunt end.

In one embodiment, the RNAi agent comprises an overhang having 1 nt to 4 nt.

In one embodiment, the RNAi agent comprises an overhang at the 3′-end of the antisense strand of the RNAi agent.

In one embodiment, the RNAi agent is ligated to one or more diagnostic compound, reporter group, cross-linking agent, nuclease-resistance conferring moiety, natural or unusual nucleobase, lipophilic molecule, cholesterol, lipid, lectin, steroid, uvaol, hecigenin, diosgenin, terpene, triterpene, sarsasapogenin, Friedelin, epifriedelanol-derivatized lithocholic acid, vitamin, carbohydrate, dextran, pullulan, chitin, chitosan, synthetic carbohydrate, oligo lactate 15-mer, natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

In one embodiment, the composition further comprises a second RNAi agent to a target described herein, e.g., PAWR. RNAi agents of the present invention can be delivered or introduced (e.g., to a cell in vitro or to a patient) by any means known in the art.“Introducing into a cell,” when referring to an iRNA, means facilitating or effecting uptake or absorption into the cell, as is understood by those skilled in the art. Absorption or uptake of an iRNA can occur through unaided diffusive or active cellular processes, or by auxiliary agents or devices. The meaning of this term is not limited to cells in vitro; an iRNA may also be “introduced into a cell,” wherein the cell is part of a living organism. In such an instance, introduction into the cell will include the delivery to the organism. For example, for in vivo delivery, iRNA can be injected into a tissue site or administered systemically. In vivo delivery can also be by a beta-glucan delivery system, such as those described in U.S. Pat. Nos. 5,032,401 and 5,607,677, and U.S. Publication No. 2005/0281781 which are hereby incorporated by reference in their entirety. In vitro introduction into a cell includes methods known in the art such as electroporation and lipofection. Further approaches are described below or known in the art.

Delivery of RNAi agent to tissue is a problem both because the material must reach the target organ and must also enter the cytoplasm of target cells. RNA cannot penetrate cellular membranes, so systemic delivery of naked RNAi agent is unlikely to be successful. RNA is quickly degraded by RNAse activity in serum. For these reasons, other mechanisms to deliver RNAi agent to target cells has been devised. Methods known in the art include but are not limited to: viral delivery (retrovirus, adenovirus, lentivirus, baculovirus, AAV); liposomes (Lipofectamine, cationic DOTAP, neutral DOPC) or nanoparticles (cationic polymer, PEl), bacterial delivery (tkRNAi), and also chemical modification (LNA) of siRNA to improve stability. Xia et al. 2002 Nat. Biotechnol. 20 and Devroe et al. 2002. BMC Biotechnol. 21: 15, disclose incorporation of siRNA into a viral vector. Other systems for delivery of RNAi agents are contemplated, and the RNAi agents of the present invention can be delivered by various methods yet to be found and/or approved by the FDA or other regulatory authorities. Liposomes have been used previously for drug delivery (e.g., delivery of a chemotherapeutic). Liposomes (e.g., cationic liposomes) are described in PCT publications W002/100435A1, W003/015757A1, and W004029213A2; U.S. Pat. Nos. 5,962,016; 5,030,453; and 6,680,068; and U.S. Patent Application 2004/0208921. A process of making liposomes is also described in W004/002453A1. Furthermore, neutral lipids have been incorporated into cationic liposomes (e.g., Farhood et al. 1995). Cationic liposomes have been used to deliver RNAi agent to various cell types (Sioud and Sorensen 2003; U.S. Patent Application 2004/0204377; Duxbury et al., 2004; Donze and Picard, 2002). Use of neutral liposomes disclosed in Miller et al. 1998, and U.S. Publ. 2003/0012812.

As used herein, the term “SNALP” refers to a stable nucleic acid-lipid particle. A SNALP represents a vesicle of lipids coating a reduced aqueous interior comprising a nucleic acid such as an iRNA or a plasmid from which an iRNA is transcribed. SNALPs are described, e.g., in U.S. Patent Application Publication Nos. 20060240093, 20070135372, and in International Application No. WO 2009082817. These applications are incorporated herein by reference in their entirety.

Chemical transfection using lipid-based, amine-based and polymer-based techniques, is disclosed in products from Ambion Inc., Austin, Tex.; and Novagen, EMD Biosciences, Inc, an Affiliate of Merck KGaA, Darmstadt, Germany); Ovcharenko D (2003) “Efficient delivery of siRNAs to human primary cells.” Ambion TechNotes 10 (5): 15-16). Additionally, Song et al. (Nat Med. published online (Fete 10, 2003) doi: 10.1038/nm828) and others [Caplen et al. 2001 Proc. Natl. Acad. Sci. (USA), 98: 9742-9747; and McCaffrey et al. Nature 414: 34-39] disclose that liver cells can be efficiently transfected by injection of the siRNA into a mammal's circulatory system.

A variety of molecules have been used for cell-specific RNAi agent delivery. For example, the nucleic acid-condensing property of protamine has been combined with specific antibodies to deliver siRNAs. Song et al. 2005 Nat Biotech. 23: 709-717. The self-assembly PEGylated polycation polyethylenimine has also been used to condense and protect siRNAs. Schiffelers et al. 2004 Nucl. Acids Res. 32: 49, 141-110.

The siRNA-containing nanoparticles were then successfully delivered to integrin overexpressing tumor neovasculature. Hu-Lieskovan et al. 2005 Cancer Res. 65: 8984-8992. The RNAi agents of the present invention can be delivered via, for example, Lipid nanoparticles (LNP); neutral liposomes (NL); polymer nanoparticles; double-stranded RNA binding motifs (dsRBMs); or via modification of the RNAi agent (e.g., covalent attachment to the dsRNA). Lipid nanoparticles (LNP) are self-assembling cationic lipid based systems. These can comprise, for example, a neutral lipid (the liposome base); a cationic lipid (for siRNA loading); cholesterol (for stabilizing the liposomes); and PEG-lipid (for stabilizing the formulation, charge shielding and extended circulation in the bloodstream). The cationic lipid can comprise, for example, a headgroup, a linker, a tail and a cholesterol tail. The LNP can have, for example, good tumor delivery, extended circulation in the blood, small particles (e.g., less than 100 nm), and stability in the tumor microenvironment (which has low pH and is hypoxic).

Neutral liposomes (NL) are non-cationic lipid based particles. Polymer nanoparticles are self-assembling polymer-based particles. Double-stranded RNA binding motifs (dsRBMs) are self-assembling RNA binding proteins, which will need modifications.

The present disclosure further provides use of a RNAi agent for the treatment of a condition associated with expression of a target protein, e.g.,a s described herein. Also provided is a use of a RNAi agent for the manufacture of a medicament for a condition associated with expression of a target protein, e.g.,a s described herein. In another embodiment, the present invention provides a method of treating a condition associated with expression of a target protein, e.g.,a s described herein, by administering to a subject in need thereof a therapeutically effective amount of a pharmaceutical composition comprising a RNAi agent described herein.

In another embodiment, a RNAi agent which inhibits the expression of a a target described herein, e.g., PAWR, for use in the treatment of a condition associated with expression of a target protein, e.g.,a s described herein is provided.

Several other molecules may be suitable to inhibit a target disclosed herein, such as low molecular weight compounds, cyclic peptides, RNAi agents, Aptamers, CRISPRs, TALENs, ZFNs, and antibodies.

### Low Molecular Weight Compounds and Therapies

In one embodiment, the disclosure comprises a low molecular weight compound inhibiting gene expression that inhibits the expression of a target described herein, e.g., PAWR.

In another embodiment, the present invention provides a molecule that inhibits the normal cellular function of the target described herein, e.g., PAWR protein.

The present disclosure thus provides use of a low molecular weight inhibitor for a target described herein, e.g., PAWR, for the treatment of a condition associated with expression of a target protein, e.g., as described herein. Also provided is a use of a low molecular weight inhibitor of a target described herein, e.g., PAWR, for the manufacture of a medicament for treating a condition associated with expression of a target protein, e.g., as described herein.

In another embodiment, the present invention provides a method of treating a condition associated with expression of a target protein, e.g.,a s described herein by administering to a subject in need thereof a therapeutically effective amount of a pharmaceutical composition comprising a RNAi agent described herein. In another embodiment, a low molecular weight inhibitor for use in the treatment of a condition associated with expression of a target protein, e.g., as described herein is provided.

The inhibitor of the present disclosure can also be, inter alia, derived from a CRISPR/Cas system, TALEN, or ZFN.

### CRISPR

By “CRISPR” or “CRISPR to a target” or “CRISPR to inhibit a target” and the like is meant a set of clustered regularly interspaced short palindromic repeats, or a system comprising such a set of repeats. By “Cas” is meant a CRISPR-associated protein. By “CRISPR/Cas” system is meant a system derived from CRISPR and Cas which can be used to silence, enhance or mutate a target described herein, e.g., the PAWR gene.

Naturally-occurring CRISPR/Cas systems are found in approximately 40% of sequenced eubacteria genomes and 90% of sequenced archaea. Grissa et al. 2007. BMC Bioinformatics 8: 172. This system is a type of prokaryotic immune system that confers resistance to foreign genetic elements such as plasmids and phages and provides a form of acquired immunity. Barrangou et al. 2007. Science 315: 1709-1712; Marragini et al. 2008 Science 322: 1843-1845. The CRISPR/Cas system has been modified for use in gene editing (silencing, enhancing or changing specific genes) in eukaryotes such as mice or primates. Wiedenheft et al. 2012. Nature 482: 331-8. This is accomplished by introducing into the eukaryotic cell a plasmid containing a specifically designed CRISPR and one or more appropriate Cas.

The CRISPR sequence, sometimes called a CRISPR locus, comprises alternating repeats and spacers. In a naturally-occurring CRISPR, the spacers usually comprise sequences foreign to the bacterium such as a plasmid or phage sequence; in the target CRISPR/Cas system, the spacers are derived from the target gene sequence. The repeats generally show some dyad symmetry, implying the formation of a secondary structure such as a hairpin, but they are not truly palindromic.

RNA from the CRISPR locus is constitutively expressed and processed by Cas proteins into small RNAs. These comprise a spacer flanked by a repeat sequence. The RNAs guide other Cas proteins to silence exogenous genetic elements at the RNA or DNA level. Horvath et al. 2010. Science 327: 167-170; Makarova et al. 2006 Biology Direct 1: 7. The spacers thus serve as templates for RNA molecules, analogously to siRNAs. Pennisi 2013. Science 341: 833-836. As these naturally occur in many different types of bacteria, the exact arrangements of the CRISPR and structure, function and number of Cas genes and their product differ somewhat from species to species. Haft et al. 2005 PLoS Comput. Biol. 1: e60; Kunin et al. 2007. Genome Biol. 8: R61; Mojica et al. 2005. J. Mol. Evol. 60: 174-182; Bolotin et al. 2005. Microbiol. 151: 2551-2561; Pourcel et al. 2005. Microbiol. 151: 653-663; and Stern et al. 2010. Trends. Genet. 28: 335-340. For example, the Cse (Cas subtype, E. coli) proteins (e.g., CasA) form a functional complex, Cascade, that processes CRISPR RNA transcripts into spacer-repeat units that Cascade retains. Brouns et al. 2008. Science 321: 960-964. In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing. Pennisi 2013. Science 341: 833-836.

The CRISPR/Cas system can thus be used to edit a target described herein, e.g., the PAWR gene (adding or deleting a basepair), e.g., repairing a damaged target gene, or introducing a premature stop which thus decreases expression of an over-expressed target. The CRISPR/Cas system can alternatively be used like RNA interference, turning off the target gene in a reversible fashion. In a mammalian cell, for example, the RNA can guide the Cas protein to the target promoter, sterically blocking RNA polymerases.

Artificial CRISPR/Cas systems can be generated which inhibit a target described herein, e.g., the PAWR gene, using technology known in the art, e.g., that described in U.S. patent application Ser. No. 13/842,859. The present disclosure thus provides a CRISPR/Cas system suitable for editing a target described herein, e.g., the PAWR gene, for use in the treatment of a condition associated with expression of a target protein, e.g., as described herein. Also provided is a use of a CRISPR/Cas system suitable for editing a target described herein, e.g., the PAWR gene for the manufacture of a medicament for treating a condition associated with expression of a target protein, e.g., as described herein. In another embodiment, the present invention provides a method of treating a condition associated with expression of a target protein, e.g., as described herein, by administering to a subject in need thereof a therapeutically effective amount of a pharmaceutical composition comprising a CRISPR/Cas system suitable for editing a target described herein, e.g., the PAWR gene.

In another embodiment, a CRISPR/Cas system suitable for editing a target described herein, e.g., the PAWR gene, for use in the treatment of a condition associated with expression of a target protein, e.g., as described herein is provided.

An inhibitory CRISPR system can include a guide RNA (gRNA) comprising a targeting domain, i.e., a nucleotide sequence that is complementary to a target DNA strand, and a second domain that interacts with an RNA-directed nuclease, e.g., cpf1 or Cas molecule, e.g., Cas9 molecule.

In some embodiments, the ability of an RNA-directed nuclease, e.g., cpf1 or Cas molecule, e.g., Cas9 molecule, to interact with and cleave a target nucleic acid is Protospacer Adjacent Motif (PAM) sequence dependent. A PAM sequence is a sequence in the target nucleic acid. In some embodiments, cleavage of the target nucleic acid occurs upstream from the PAM sequence. RNA-directed nuclease molecules, e.g., cpf1 or Cas molecules, e.g., Cas9 molecules, from different bacterial species can recognize different sequence motifs (e.g., PAM sequences). In addition to recognizing different PAM sequences, RNA-directed nucleases, e.g., cpf1 or Cas molecules, e.g., Cas9 molecules, from different species may be directed to different target sequences (e.g., target sequences adjacent, e.g., immediately upstream, to the PAM sequence) by gRNA molecules comprising targeting domains capable of hybridizing to said target sequences and a tracr sequence that binds to said RNA-directed nuclease, e.g., cpf1 or Cas molecule, e.g., Cas9 molecule.

In some embodiments, the CRISPR system comprises a gRNA molecule and a Cas9 molecule from S. pyogenes. A Cas9 molecule of S. pyogenes recognizes the sequence motif NGG and directs cleavage of a target nucleic acid sequence 1 to 10, e.g., 3 to 5, base pairs upstream from that sequence.

In some embodiments, the CRISPR system comprises a gRNA molecule and a Cas9 molecule from S. thermophilus. A Cas9 molecule of S. thermophilus recognizes the sequence motif NGGNG and NNAGAAW (W=A or T) and directs cleavage of a core target nucleic acid sequence 1 to 10, e.g., 3 to 5, base pairs upstream from these sequences. A gRNA molecule useful with S. thermophilus-based CRISPR systems may include a tracr sequence known to interact with S. thermophilus. See, e.g., Horvath et al., SCIENCE 2010; 327(5962): 167-170, and Deveau et al., J BACTERIOL 2008; 190(4): 1390-1400.

In some embodiments, the CRISPR system comprises a gRNA molecule and a Cas9 molecule from S. aureus. A Cas9 molecule of S. aureus recognizes the sequence motif NNGRR (R=A or G) and directs cleavage of a target nucleic acid sequence 1 to 10, e.g., 3 to 5, base pairs upstream from that sequence.

In some embodiments, the CRISPR system comprises a gRNA molecule and a RNA-directed nuclease, e.g., cpf1 molecule, e.g., a cpf1 molecule from L. bacterium or a cpf1 molecule from A. sp. A cpf1 molecule, e.g., a cpf1 molecule from L. bacterium or a cpf1 molecule from A. sp., recognizes the sequence motive of TTN (where N=A, T, G or C) or preferably TTTN (where N=A, T, G or C), and directs cleavage of a target nucleic acid sequence 1-25 base pairs upstream of the PAM sequence, e.g., 18-19 base pairs upstream from the PAM sequence on the same strand as the PAM and 23 base pairs upstream of the PAM sequence on the opposite strand as the PAM, creating a sticky end break.

### TALEN

By “TALEN” or “TALEN to target” or “TALEN to inhibit target” and the like is meant a transcription activator-like effector nuclease, an artificial nuclease which can be used to edit a target described herein, e.g., the PAWR gene.

TALENs are produced artificially by fusing a TAL effector DNA binding domain to a DNA cleavage domain. Transcription activator-like effects (TALEs) can be engineered to bind any desired DNA sequence, including a portion of the target gene. By combining an engineered TALE with a DNA cleavage domain, a restriction enzyme can be produced which is specific to any desired DNA sequence, including a sequence to a target described herein, e.g., a PAWR target sequence. These can then be introduced into a cell, wherein they can be used for genome editing. Boch 2011 Nature Biotech. 29: 135-6; and Boch et al. 2009 Science 326: 1509-12; Moscou et al. 2009 Science 326: 3501.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

To produce a TALEN, a TALE protein is fused to a nuclease (N), which is a wild-type or mutated FokI endonuclease. Several mutations to FokI have been made for its use in TALENs; these, for example, improve cleavage specificity or activity. Cermak et al. 2011 Nucl. Acids Res. 39: e82; Miller et al. 2011 Nature Biotech. 29: 143-8; Hockemeyer et al. 2011 Nature Biotech. 29: 731-734; Wood et al. 2011 Science 333: 307; Doyon et al. 2010 Nature Methods 8: 74-79; Szczepek et al. 2007 Nature Biotech. 25: 786-793; and Guo et al. 2010 J. Mol. Biol. 200: 96.

The FokI domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. Miller et al. 2011 Nature Biotech. 29: 143-8.

A TALEN to a target can be used inside a cell to produce a double-stranded break (DSB). A mutation can be introduced at the break site if the repair mechanisms improperly repair the break via non-homologous end joining. For example, improper repair may introduce a frame shift mutation. Alternatively, foreign DNA can be introduced into the cell along with the TALEN; depending on the sequences of the foreign DNA and chromosomal sequence, this process can be used to correct a defect in the target gene or introduce such a defect into a wt target gene, thus decreasing expression of target gene.

TALENs specific to sequences in a target described herein, e.g., the PAWR gene, can be constructed using any method known in the art, including various schemes using modular components. Zhang et al. 2011 Nature Biotech. 29: 149-53; Geibler et al. 2011 PLoS ONE 6: e19509.

The present disclosure thus provides use of a TALEN for a target described herein, e.g., PAWR, for the treatment of a condition associated with expression of a target protein, e.g., as described herein. Also provided is a use of a TALEN for the manufacture of a medicament for treating a condition associated with expression of a target protein, e.g., as described herein.

In another embodiment, the present invention provides a method of a condition associated with expression of a target protein, e.g., as described herein, by administering to a subject in need thereof a therapeutically effective amount of a pharmaceutical composition comprising a TALEN of a a target described herein, e.g., PAWR. In another embodiment, a TALEN of a target described herein, e.g., PAWR for use in the treatment of a condition associated with expression of a target protein, e.g., as described herein is provided.

### Zinc Finger Nuclease

By “ZFN” or “Zinc Finger Nuclease” or “ZFN to a target gene” or “ZFN to inhibit target gene” and the like is meant a zinc finger nuclease, an artificial nuclease which can be used to edit a target described herein, e.g., the PAWR gene.

Like a TALEN, a ZFN comprises a FokI nuclease domain (or derivative thereof) fused to a DNA-binding domain. In the case of a ZFN, the DNA-binding domain comprises one or more zinc fingers. Carroll et al. 2011. Genetics Society of America 188: 773-782; and Kim et al. Proc. Natl. Acad. Sci. USA 93: 1156-1160.

A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

Like a TALEN, a ZFN must dimerize to cleave DNA. Thus, a pair of ZFNs is required to target non-palindromic DNA sites. The two individual ZFNs must bind opposite strands of the DNA with their nucleases properly spaced apart. Bitinaite et al. 1998 Proc. Natl. Acad. Sci. USA 95: 10570-5.

Also like a TALEN, a ZFN can create a double-stranded break in the DNA, which can create a frame-shift mutation if improperly repaired, leading to a decrease in the expression and amount of a target in a cell. ZFNs can also be used with homologous recombination to mutate, or repair defects, in the target gene.

ZFNs specific to sequences in a target described herein, e.g., the PAWR gene, can be constructed using any method known in the art. Cathomen et al. Mol. Ther. 16: 1200-7; and Guo et al. 2010. J. Mol. Biol. 400: 96.

The present disclosure thus provides use of a ZFN specific to sequences in a target described herein, e.g., the PAWR gene for the treatment of a condition associated with expression of a target protein, e.g., as described herein. Also provided is a use of a ZFN specific to sequences in a target described herein, e.g., the PAWR gene for the manufacture of a medicament a condition associated with expression of a target protein, e.g., as described herein.

In another embodiment, the present invention provides a method of treating a condition associated with expression of a target protein, e.g., as described herein, by administering to a subject in need thereof a therapeutically effective amount of a pharmaceutical composition comprising a ZFN specific to sequences in a target described herein, e.g., the PAWR gene. In another embodiment, a ZFN specific to sequences in a target described herein, e.g., the PAWR gene, for use in the treatment of a condition associated with expression of a target protein, e.g., as described herein is provided.

### Antibodies

In some embodiments, the present invention provides an inhibitor, e.g., an inhibitor of a target described herein, e.g., a PAWR inhibitor, which is an antibody or epitope-binding fragment or derivative thereof, and methods of using the same. Various types of antibodies and epitope-binding fragments and derivatives thereof are known in the art, as are methods of producing these. Any of these, including but not limited to those described herein, can be used to produce an inhibitor of a target described herein, e.g., PAWR, which can be used in various methods of inhibiting the target and treating a condition associated with expression of a target protein, e.g., as described herein.

In certain embodiments of the invention, the antibody to a target described herein, e.g., PAWR, is an intrabody. Single chain antibodies expressed within the cell (e.g. cytoplasm or nucleus) are called intrabodies. Due to the reducing environment within the cell, disulfide bridges, believed to be critical for antibody stability, are not formed. Thus, it was initially believed that applications of intrabodies are not suitable. But several cases are described showing the feasibility of intrabodies (Beerli et al., 1994 J Biol Chem, 269, 23931-6; Biocca et al., 1994 Bio/Technology, 12, 396-9; Duan et al., 1994 Proceedings of the National Academy of Sciences of the United States of America, 91, 5075-9; Gargano and Cattaneo, 1997 FEBS Lett, 414, 537-40; Greenman et al., 1996 J Immunol Methods, 194, 169-80; Martineau et al., 1998 Journal of Molecular Biology, 280, 117-27; Mhashilkar et al., 1995 EMBO Journal, 14, 1542-51; Tavladoraki et al., 1993 Nature, 366, 469-72). In these cases, intrabodies work by, e.g., blocking the cytoplasmic antigen and therefore inhibiting its biological activity.

Such intracellular antibodies are also referred to as intrabodies and may comprise a Fab fragment, or preferably comprise a scFv fragment (see, e.g., Lecerf et al., Proc. Natl. Acad. Sci. USA 98:4764-49 (2001). The framework regions flanking the CDR regions can be modified to improve expression levels and solubility of an intrabody in an intracellular reducing environment (see, e.g., Worn et al., J. Biol. Chem. 275:2795-803 (2000). An intrabody may be directed to a particular cellular location or organelle, for example by constructing a vector that comprises a polynucleotide sequence encoding the variable regions of an intrabody that may be operatively fused to a polynucleotide sequence that encodes a particular target antigen within the cell (see, e.g., Graus-Porta et al., Mol. Cell Biol. 15:1182-91 (1995); Lener et al., Eur. J. Biochem. 267:1196-205 (2000)). An intrabody may be introduced into a cell by a variety of techniques available to the skilled artisan including via a gene therapy vector, or a lipid mixture (e.g., Provectin.™. manufactured by Imgenex Corporation, San Diego, Calif.), or according to photochemical internalization methods.

Intrabodies can be derived from monoclonal antibodies which were first selected with classical techniques (e.g., phage display) and subsequently tested for their biological activity as intrabodies within the cell (Visintin et al., 1999 Proceedings of the National Academy of Sciences of the United States of America, 96, 11723-11728). For additional information, see: Cattaneo, 1998 Bratisl Lek Listy, 99, 413-8; Cattaneo and Biocca, 1999 Trends In Biotechnology, 17, 115-21. The solubility of an intrabody can be modified by either changes in the framework (Knappik and Pluckthun, 1995 Protein Engineering, 8, 81-9) or the CDRs (Kipriyanov et al., 1997; Ulrich et al., 1995 Protein Engineering, 10, 445-53). Additional methods for producing intrabodies are described in the art, e.g., U.S. Pat. Nos. 7,258,985 and 7,258,986.

In one embodiment, antigen-binding proteins, such as antibodies, that are able to target cytosolic/intracellular proteins, for example, a target described herein, e.g., a PAWR protein. The disclosed antibodies target a peptide/MHC complex as it would typically appear on the surface of a cell following antigen processing of the target protein and presentation by the cell. HLA class I binds to peptides approximately 9 amino acids in length and presents them on the surface of the cell to cytotoxic T lymphocytes. The presentation of these peptides is the product of cytoplasmic cleavage by enzymes and active transport by transporter proteins. Further, the binding of particular peptides after processing and localization is heavily influenced by the amino acid sequence of the particular HLA protein. Most of these steps are amenable to in vitro characterization, allowing one to predict the likelihood that a particular amino acid sequence, derived from a larger peptide or protein of interest, will be successfully processed, transported, bound by MHC class I, and presented to cytotoxic T lymphocytes. In that regard, the antibodies mimic T-cell receptors in that the antibodies have the ability to specifically recognize and bind to a peptide in an MHC-restricted fashion, that is, when the peptide is bound to an MHC antigen. The peptide/MHC complex recapitulates the antigen as it would typically appear on the surface of a cell following antigen processing and presentation of the target protein to a T-cell.

The accurate prediction for a particular step in this process is dependent upon models informed by experimental data. The cleavage specificity of the proteasome, producing peptides often <30 amino acids in length, can be determined by in vitro assays. The affinity for the transporter complex can similarly be determined by relatively straight-forward in vitro binding assays. The MHC class I protein's affinity is highly variable, depending on the MHC allele, and generally must be determined on an allele-by-allele basis. One approach is to elute the peptides presented by the MHC protein on the cell surface to generate a consensus motif. An alternative approach entails generating cells deficient in a peptide processing step such that most or all of the MHC proteins on the cell surface are not loaded with a peptide. Many different peptides can be washed over the cells in parallel and monitored for binding. The set of peptides that do and do not bind can be used to train a classifier (such as an artificial neural network or support vector machine) to discriminate between the two peptide sets. This trained classifier can then be applied to novel peptides to predict their binding to the MHC allele. Alternatively, the affinity for each peptide can be used to train a regression model, which can then be used to make quantitative predictions regarding the MHC protein's affinity for an untested peptide. The collection of such datasets is laborious, so methods exist to combine data collected for one HLA allele with the knowledge of the amino acid differences between that particular allele and another unstudied MHC allele to predict its peptide binding specificity.

Additional methods for constructing antibodies to cytosolic peptides to a target described herein, e.g., such as PAWR, are disclosed for example in, WO 2012/135854, which is hereby incorporated by reference in its entirety. This document describes production of antibodies which recognize and bind to epitopes of a peptide/MHC complex, such as a peptide/HLA-A2 or peptide/HLA-A0201 complex. In some embodiments of the invention, the peptide is portion of a target described herein, e.g., PAWR gene.

HLA class I binds to peptides approximately 9 amino acids in length and presents them on the surface of the cell to cytotoxic T lymphocytes. The presentation of these peptides is the product of cytoplasmic cleavage by enzymes and active transport by transporter proteins. Further, the binding of particular peptides after processing and localization is heavily influenced by the amino acid sequence of the particular HLA protein. Most of these steps are amenable to in vitro characterization, allowing one to predict the likelihood that a particular amino acid sequence, derived from a larger peptide or protein of interest, will be successfully processed, transported, bound by MHC class I, and presented to cytotoxic T lymphocytes.

One such machine learning approach that combines prediction of likely proteasomal cleavage, transporter affinity, and MHC affinity is SMM (Stabilized Matrix Method, Tenzer S et al, 2005. PMID 15868101). This approach can be extended to mutations specific to an indication: a mutation leading to an amino acid change alters the peptide sequence and can lead to a peptide that produces a different score than the wildtype sequence. By focusing on such mutations and selecting those mutant peptide sequences that score highly, one can generate peptides that are presented solely in a diseased state because the sequence simply does not exist in a non-diseased individual. Cross-reactivity can be further minimized by also evaluating the wildtype sequence and selecting for downstream analyses only those peptides whose non-mutant sequence is not predicted to be processed and presented by MHC efficiently.

Once appropriate peptides have been identified, peptide synthesis may be done in accordance with protocols well known to those of skill in the art. Peptides may be directly synthesized in solution or on a solid support in accordance with conventional techniques (See for example, Solid Phase Peptide Synthesis by John Morrow Stewart and Martin et al. Application of Almez-mediated Amidation Reactions to Solution Phase Peptide Synthesis, Tetrahedron Letters Vol. 39, pages 1517-1520 1998.). Peptides may then be purified by high-pressure liquid chromatography and the quality assessed by high-performance liquid chromatography analysis. Purified peptides may be dissolved in DMSO diluted in PBS (pH7.4) or saline and stored at −80 C. The expected molecular weight may be confirmed using matrix-assisted laser desorption mass spectrometry.

Subsequent to peptide selection, binding of the peptide to HLA-A may be tested. In one method, binding activity is tested using the antigen-processing deficient T2 cell line, which stabilizes expression of HLA-A on its cell surface when a peptide is loaded exogenously in the antigen-presenting groove by incubating the cells with peptide for a sufficient amount of time. This stabilized expression is read out as an increase in HLA-A expression by flow cytometry using HLA-A2 specific monoclonal antibodies (for example, BB7.2) compared to control treated cells. In another method, presence of the peptide in the HLA-A2 antigen-presenting groove of T2 cells may be detected using targeted mass spectrometry. The peptides are enriched using a MHC-specific monoclonal Ab (W6/32) and then specific MRM assays monitor the peptides predicted to be presented (See for example, Kasuga, Kie. (2013) Comprehensive Analysis of MHC Ligands in Clinical material by Immunoaffinity-Mass Spectrometry, Helena Backvall and Janne Lethio, The Low Molecular Weight Proteome: Methods and Protocols (203-218), New York, N.Y.: Springer Sciences+Business Media and Kowalewski D and Stevanovic S. (2013) Biochemical Large-Scale Identification of MHC Class I Ligands, Peter van Endert, Antigen Processing: Methods and Protocols, Methods in Molecular Biology, Vol 960 (145-158), New York, N.Y.: Springer Sciences+Business Media). This strategy differs slightly than the normally applied tandem mass spectrometry based peptide sequencing. Heavy labeled internal standards are used for identification which results in a more sensitive and quantitative approach.

Once a suitable peptide has been identified the next step would be identification of specific antibodies to the peptide/HLA-A complex, the “target antigen”, utilizing conventional antibody generation techniques such as phage display or hybridoma technology in accordance with protocols well known to those skilled in the art. The target antigen (for example, the peptide/HLA-A02-01 complex) is prepared by bringing the peptide and the HLA-A molecule together in solution to form the complex. Next, selection of Fab or scFv presenting phage that bind to the target antigen are selected by iterative binding of the phage to the target antigen, which is either in solution or bound to a solid support (for example, beads or mammalian cells), followed by removal of non-bound phage by washing and elution of specifically bound phage. The targeted antigen may be first biotinylated for immobilization, for example, to streptavidin-conjugated (for example, Dynabeads M-280).

Positive Fab or scFv clones may be then tested for binding to peptide/HLA-A2 complexes on peptide-pulsed T2 cells by flow cytometry. T2 cells pulsed with the specific peptide or a control irrelevant peptide may be incubated with phage clones. The cells are washed and bound phage are detected by binding an antibody specific for the coat protein (for example, M13 coat protein antibody) followed by a fluorescent labelled secondary antibody to detect the coat protein antibody (for example, anti-mouse Ig). Binding of the antibody clones to human tumor cells expressing both HLA-A2 and the target can also be assessed by incubating the tumor cells with phage as described or purified Fab or scFv flow cytometry and appropriate secondary antibody detection.

An alternative method to isolating antibodies specific to the peptide/HLA-A2 complex may be achieved through conventional hybridoma approaches in accordance with protocols well known to those of skill in the art. In this method, the target antigen is injected into mice or rabbits to elicit an immune response and monoclonal antibody producing clones are generated. In one embodiment, the host mouse may be one of the available human HLA-A2 transgenic animals which may serve to reduce the abundance of non-specific antibodies generated to HLA-A2 alone. Clones may then be screened for specific binding to the target antigen using standard ELISA methods (for example, incubating supernatant from the clonal antibody producing cells with biotinylated peptide/MHC complex captured on streptavidin coated ELISA plates and detected with anti-mouse antibodies). The positive clones can also be identified by incubating supernatant from the antibody producing clones with peptide pulsed T2 cells by flow cytometry and detection with specific secondary antibodies (for example, fluorescent labelled anti-mouse IgG antibodies). Binding of the antibody clones to human tumor cells expressing both HLA-A2 and the target can also be assessed by incubating the tumor cells with supernatant or purified antibody from the hybridoma clones by flow cytometry and appropriate secondary antibody detection.

### Screening for Degrons

Provided herein, inter alia, is a method of screening for degrons (e.g., degradation domains), or a method of selecting a degron. In an embodiment, the method comprises providing human pluripotent stem cells (hPSCs) expressing a fusion protein comprising a candidate degron and PAWR; and selecting the degron when the fusion protein decreases dissociation-induced death (DID) of the hPSCs, as compared to a population of hPSCs not expressing the degron.

Provided herein are also a method of screening for compounds that regulate a degron, or a method of selecting a compound that regulates a degron. In some embodiments, the methods comprise providing hPSCs expressing a fusion protein comprising a degron and PAWR; treating the hPSCs with a candidate compound that regulates the degron; and selecting the compound when treatment of the compound inreases or decreases dissociation-induced death (DID) of the hPSCs, as compared to a population of hPSCs not treated with the compound. In some embodiments, the compound can be a stabilization compound or destabilization compound. In some embodiments, if treatment of the compound increases DID of the hPSCs, e.g., as described herein, the compound can be selected as a stabilization compound. In some embodiments, if treatment of the compound decreases DID of the hPSCs, e.g., as described herein, the compound can be selected as a destabilization compound.

In some embodiments, a degradation domain, or degron, is a domain that assumes a stable conformation when expressed in the presence of a stabilization compound, e.g., a stabilization compound described herein. In some embodiments, a degradation domain, or degron, is a domain that assumes a stable conformation when expressed in the absence of a destabilization compound, e.g., a destabilization compound described herein.

In some embodiments, a fusion protein described herein comprises a degron, e.g., a candidate degron, and PAWR molecule, e.g., a fragment of PAWR or full-length PAWR. In some embodiments, hPSCs cultured by any of the methods described herein can be modified, e.g., by a method described herein, to express a fusion protein comprising a degron, e.g., a candidate degron and PAWR molecule, e.g., a fragment of PAWR or full-length PAWR. In some embodiments, modified hPSCs can be cultured in the presence or absence of a stabilization compound, e.g., as described herein.

Without wishing to be bound by theory, it is believed that in some embodiments, a degron can be selected, e.g., identified, by selecting for hPSCs (e.g., hPSCs which have been modified to express a fusion protein comprising a candidate degron and a PAWR molecule), which show reduced dissociation-induced death. In some embodiments, reduced DID in the hPSCs is due to degradation of PAWR by the degron, e.g., by targeting PAWR for proteasomal degradation.

In some embodiments, hPSCs used in a screen to select a degron as described herein are modified, e.g., by a method described herein, to express a fusion protein comprising a degron, e.g., a candidate degron, and a PAWR molecule, e.g., a fragment of PAWR or full-length PAWR. In some embodiments, the hPSCs are modified by contacting the population of hPSCs with a nucleotide encoding a fusion protein, e.g., a plurality of nucleotides encoding distinct fusion proteins, e.g., a library of fusion proteins. In some embodiments, the library of fusion proteins encoded by the nucleotides comprises a plurality of distinct fusion proteins, e.g., encoding a different candidate degron. In some embodiments, hPSCs contacted with nucleotides comprising a library of fusion proteins expresses only one, e.g., a distinct fusion protein, e.g., a fusion protein comprising a distinct candidate degron. In some embodiments, a population of hPSCs, e.g., comprising hPSCs expressing distinct fusion proteins, e.g., a fusion proteins comprising distinct candidate degrons, can be used in a screen as described herein. In some embodiments, the distinct fusion proteins, e.g., fusion proteins comprising distinct candidate degrons, comprise PAWR molecule, e.g., a fragment of PAWR or full-length PAWR.

In some embodiments, the nucleotides comprising the library of fusion proteins further comprises a tag, e.g., a unique identifier tag, e.g., a unique nuelcotide tag comprising at least 3, 4, 5, 6, 7, 8, 9, 10, 15, 20, or 30 nucleotides. These unique tags can be used in the identification of candidate degrons obtained from a screen, e.g., a screen to select degrons as described herein.

In some embodiments, hPSCs modified to express a fusion protein comprising a candidate degron and a PAWR molecule, e.g., a fragment of PAWR or full-length PAWR, are not contacted with a stabilization compound, e.g., as described herein. In some embodiments, the screen is performed in the absence of the stabilization compound. Without wishing to be bound by theory, it is believed that in some embodiments, the absence of the stabilization compound results in degradation of PAWR by the degron, e.g., by targeting PAWR for proteasomal degradation.

In some embodiments, hPSCs modified to express a fusion protein comprising a candidate degron and PAWR, e.g., a fragment of PAWR or full-length PAWR, are contacted with a stabilization compound, e.g., as described herein. Without wishing to be bound by theory, it is believed that in some embodiments, the presence of the stabilization compound does not result in degradation of PAWR.

In some embodiments, the fusion protein further comprises a protease cleavage site, e.g., a furin cleavage site. In some embodiments, the cleavage site is cleaved by a protease selected from the group consisting of furin, PCSK1, PCSK5, PCSK6, PCSK7, cathepsin B, Granzyme B, Factor XA, Enterokinase, genenase, sortase, precission protease, thrombin, TEV protease, and elastase 1. Exemplary sequences of protease cleavage sites, including furin cleavage sites are disclosed in International Application WO 2017/181119 filed on 14 Apr. 2017, the entire contents of which is hereby expressly incorporated by reference.

In some embodiments, hPSCs modified to express a fusion protein comprising a candidate degron and PAWR, e.g., a fragment of PAWR or full-length PAWR, have been previously modified to not express endogenous PAWR, e.g., by a method described herein, e.g., CRISPR/Cas9.

In some embodiments, the hPSCs are cultured in the absence of a DID inhibitor, e.g., a DID inhibitor described herein. In other embodiments, the hPSCs have reduced DID compared to hPSCs that have not been modified to express a fusion protein comprising a candidate degron and PAWR. In some embodiments, the hPSCs have reduced DID, e.g., a similar reduction in DID compared to hPSCs cultured in the presence of a DID inhibitor, e.g., a DID inhibitor described herein.

Methods of generating degradation domains that are selectively stable in the presence of a stabilization compound are well known in the art and discussed further below. Several such domain-stabilization compound pairs have been generated to date and are featured in the present invention. These include degradation domains based on FKBP (e.g., using a “Shield” stabilization compound) as described in: A Rapid, Reversible, and Tunable Method to Regulate Protein Function in Living Cells Using Synthetic Small Molecules.” Banaszynski, L. A.; Chen, L.-C.; Maynard-Smith, L. A.; Ooi, A. G. L.; Wandless, T. J. Cell, 2006, 126, 995-1004; domains based on DHFR (e.g., using trimethoprim as a stabilization compound) as described in A general chemical method to regulate protein stability in the mammalian central nervous system. Iwamoto, M.; Bjorklund, T.; Lundberg, C.; Kirik, D.; Wandless, T. J. Chemistry & Biology, 2010, 17, 981-988; and domains based on estrogen receptor alpha (e.g., where 4OHT is used as a stabilization compound) as described in Destabilizing domains derived from the human estrogen receptor Y Miyazaki, H Imoto, L-c Chen, T J Wandless J. Am. Chem. Soc. 2012, 134, 3942-3945. Each of these references is incorporated by reference in its entirety.

The present disclosure encompasses degradation domains derived from any naturally occurring protein. Preferably, fusion proteins of the invention will include a degradation domain for which there is no ligand natively expressed in the cell compartments of interest. For example, if the fusion protein is designed for expression in T-cells, it is preferable to select a degradation domain for which there is no naturally occurring ligand present in T cells. Thus, the degradation domain, when expressed in the cell of interest, will only be stabilized in the presence of an exogenously added compound. Notably, this property can be engineered by either engineering the degradation domain to no longer bind a natively expressed ligand (in which case the degradation domain will only be stable in the presence of a synthetic compound) or by expressing the degradation domain in a compartment where the natively expressed ligand does not occur (e.g., the degradation domain can be derived from a species other than the species in which the fusion protein will be expressed).

Degradation domain-stabilization compound pairs can be derived from any naturally occurring or synthetically developed protein. Stabilization compounds can be any naturally occurring or synthetic compounds. In certain embodiments, the stabilization compounds will be existing prescription or over-the-counter medicines.

Examples of proteins that can be engineered to possess the properties of a degradation domain along with a corresponding stabilization compound are set forth in Table 21 of International Application WO 2017/181119 filed on 14 Apr. 2017, the entire contents of which is hereby expressly incorporated by reference.

Exemplary degrons derived from the Ikaros family of transcription factors, e.g., IKZF1 or IKZF3, is disclosed in Kronke, J. et al. (2014) Science 343(6168):301-5), the entire contents of which is hereby expressly incorporated by reference.

In some embodiments, a candidate degron comprises:

a furin degron (FurON) domain;

a degron derived from an FKB protein (FKBP);

a degron derived from dihydrofolate reductase (DHFR);

a degron derived from an estrogen receptor (ER);

a degron derived from IKZF1, or IKZF3, e.g., as disclosed in Kronke, J. et al. (2014) Science 343(6168):301-5); or

a degron derived from a protein listed in Table 21 of International Application WO 2017/181119 filed on 14 Apr. 2017.

In some embodiments, the degradation domain is derived from an estrogen receptor (ER). In some embodiments, the degradation domain comprises an amino acid sequence selected from SEQ ID NO: 58 of WO 2017/181119, or a sequence having at least 90%, 95%, 97%, 98%, or 99% identity thereto, or SEQ ID NO: 121 of WO 2017/181119, or a sequence having at least 90%, 95%, 97%, 98%, or 99% identity thereto. In some embodiments, the degradation domain comprises an amino acid sequence selected from SEQ ID NO: 58, or SEQ ID NO: 121 of WO 2017/181119. When the degradation domain is derived from an estrogen receptor, the stabilization compound can be selected from Bazedoxifene or 4-hydroxy tamoxifen (4-OHT). In some embodiments, the stabilization compound is Bazedoxifene. Tamoxifen and Bazedoxifene are FDA approved drugs, and thus are safe to use in human.

In some embodiments, the degradation domain is derived from an FKB protein (FKBP). In some embodiments, the degradation domain comprises an amino acid sequence of SEQ ID NO: 56 of WO 2017/181119, or a sequence having at least 90%, 95%, 97%, 98%, or 99% identity thereto. In some embodiments, the degradation domain comprises an amino acid sequence of SEQ ID NO: 56 of WO 2017/181119. When the degradation domain is derived from a FKBP, the stabilization compound can be Shield-1.

In some embodiments, the degradation domain is derived from dihydrofolate reductase (DHFR). In some embodiments, the degradation domain comprises an amino acid sequence selected from SEQ ID NO: 57 of WO 2017/181119, or a sequence having at least 90%, 95%, 97%, 98%, or 99% identity thereto. In some embodiments, the degradation domain comprises an amino acid sequence selected from SEQ ID NO: 57 of WO 2017/181119. When the degradation domain is derived from a DHFR, the stabilization compound can be Trimethoprim.

Additional exemplary proteins for generating degradation domains is described in Table 21 on pages 210-220 of International Application WO 2017/181119 filed on 14 Apr. 2017, the entire contents of which is hereby expressly incorporated by reference.

Additional exemplary degrons are described in International Patent Publication No. WO2017/024318, the entire contents of which is hereby expressly incorporated by reference.

### Target Proteins and Indications

In some embodiments, hPSCs used in any of the methods disclosed herein can be undifferentiated or differentiated, e.g., differentiated into a specific cell type, e.g., as disclosed herein. In some embodiments, hPSCs differentiated into a specific cell type may display a high nuclear/cytoplasmic ratios and prominent nucleoli. In some embodiments, hPSCs may be cultured in the presence of suitable nutrients and optionally other cells such that the hPSCs can grow and optionally differentiate.

In some embodiments, a hPSC can be differentiated, e.g., into a target cell, e.g., a target cell from any organ or tissue described herein, e.g., a cardiomyocyte. In some embodiments, a hPSC can be differentiated into a target cell by modifying the hPSC by any of the methods of modifying a hPSC as described herein. In some embodiments, the hPSC is differentiated into a target cell by expressing a target protein, e.g., Wnt3a, by any of the methods described herein.

In some embodiments, a hPSC differentiated into a target cell, e.g., a target cell from any organ or tissue described herein can be used to identify gene targets for new drugs, or to test toxicity or teratogenicity of new compounds.

In some embodiments, a hPSC differentiated into a target cell, e.g., a target cell from any organ or tissue described herein can be used as a therapy, e.g., a treatment, for transplantation to replace cell populations in a condition, e.g., a condition associated with expression of a target protein as described herein. In some embodiments, the condition can include, but is not limited to neurodegenerative or neuroinflammatory diseases (e.g., Parkinson's disease, amyotrophic lateral sclerosis, Alzheimer's disease, multiple sclerosis, Huntington's disease, frontotemporal dementia (FTD), progressive supranuclear palsy, Nasu-Hakola disease, anti-NMDA receptor encephalitis, autism, brain lupus (NP-SLE), chemo-induced peripheral neuropathy (CIPN), postherapeutic neuralgia, chronic inflammatory demyelinating polyneuropathy (CIDP), epilepsy, Guillain-Barre Syndrom (GBS), inclusion body myositis, lysosomal storage diseases, sphingomyelinlipidose (Niemann-Pick C), mucopolysaccharidose II/IIIB, metachromatic leukodystrophy, multifocal motor neuropathy, Myasthenia Gravis, Neuro-Behcet's Disease, neuromyelitis optica (NMO), optic neuritis, polymyositis, dermatomyositis, Rasmussen's encephalitis, Rett's Syndrome, stroke, transverse myelitis, traumatic brain injury, spinal cord injury, viral encephalitis, bacterial meningitis, brain and spinal cord injury, cardiac conditions (e.g., myocardial infarction), conditions associated with abnormal hematopoietic cell formation, wound healing, teeth regeneration, hair regeneration, blindness and vision impairment, metabolic disorders (e.g., pancreatic beta cell regeneration in e.g., diabetes, e.g., juvenile-onset diabetes mellitus), and proliferative disorders, e.g., cancers.

In some embodiments, the condition is a cancer, e.g., a hematological cancer (e.g., a leukemia or lymphoma), or a solid tumor. In some embodiments, the cancer is selected from the group consisting of one or more acute leukemias including but not limited to B-cell acute lymphoid leukemia (“BALL”), T-cell acute lymphoid leukemia (“TALL”), acute lymphoid leukemia (ALL); one or more chronic leukemias including but not limited to chronic myelogenous leukemia (CML), chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and any combination thereof. In some embodiments, the cancer is a solid tumor, e.g., a solid tumor described herein, e.g., prostatic, colorectal, pancreatic, cervical, gastric, ovarian, head, or lung cancer.

In some embodiments, hPSCs disclosed herein are modified to express a target protein, e.g., Wnt3a. In some embodiments, hPSCs modified to express Wnt3a can be differentiated into a target cell, e.g., cardiomyocytes. In some embodiments, hPSCs modified to express Wnt3a can be used to treat a cardiac condition, e.g., a myocardial infarction. In some embodiments, the hPSCs modified to express Wnt3a used to treat the cardiac condition may or may not have been differentiated into a target cell, e.g., cardiomyoctes.

### Sample Preparation

In some embodiments, hPSCs can be isolated from a sample, e.g., a sample from an organ or tissue, e.g., blood, skin, bone marrow, ovarian epithelium, testis, skeletal muscle, teeth, gut, liver, or brain, from a subject, e.g., a subject described herein.

Body fluid samples can be obtained from a subject using any of the methods known in the art.

Generally, a sample of an organ or tissue can be a test sample of cells or tissue that are obtained from a subject by biopsy or surgical resection. A sample of cells or tissue can be removed by needle aspiration biopsy. For this, a fine needle attached to a syringe is inserted through the skin and into the tissue of interest. The needle is typically guided to the region of interest using ultrasound or computed tomography (CT) imaging. Once the needle is inserted into the tissue, a vacuum is created with the syringe such that cells or fluid may be sucked through the needle and collected in the syringe. A sample of cells or tissue can also be removed by incisional or core biopsy. For this, a cone, a cylinder, or a tiny bit of tissue is removed from the region of interest. CT imaging, ultrasound, or an endoscope is generally used to guide this type of biopsy. In some embodiments, hPSCs can then be isolated from the sample using methods known in the art.

In some embodiments, the test sample of, for example tissue, may also be stored in, e.g., RNAlater (Ambion; Austin Tex.) or flash frozen and stored at −80° C. for later use. The biopsied tissue sample may also be fixed with a fixative, such as formaldehyde, paraformaldehyde, or acetic acid/ethanol. The fixed tissue sample may be embedded in wax (paraffin) or a plastic resin. The embedded tissue sample (or frozen tissue sample) may be cut into thin sections. RNA or protein may also be extracted from a fixed or wax-embedded tissue sample.

### Measurement of Gene Expression

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips.

In one aspect, gene expression is detected and quantitated by hybridization to a probe that specifically hybridizes to the appropriate probe for that biomarker. The probes also can be attached to a solid support for use in high throughput screening assays using methods known in the art. WO 97/10365 and U.S. Pat. Nos. 5,405,783, 5,412,087 and 5,445,934, for example, disclose the construction of high density oligonucleotide chips which can contain one or more of the sequences disclosed herein. Using the methods disclosed in U.S. Pat. Nos. 5,405,783, 5,412,087 and 5,445,934, the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

In one aspect, the expression level of a gene is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device. See U.S. Pat. Nos. 5,578,832 and 5,631,734. Alternatively any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg²⁺ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination. In one embodiment, the hybridized nucleic acids are detected by detecting one or more labels attached to the sample nucleic acids. The labels can be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g. fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, Texas red, rhodamine, green fluorescent protein, and the like), radiolabels (e.g., 3H, 125I, 35S, 14C, or 32P) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the coloured label.

The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

### Detection of Polypeptides

Expression level of PAWR or any of the stem cell associated markers described herein, can be determined by examining protein expression or the protein product. Determining the protein level involves measuring the amount of any immunospecific binding that occurs between an antibody that selectively recognizes and binds to the polypeptide of the biomarker in a sample obtained from a patient and comparing this to the amount of immunospecific binding of at least one biomarker in a control sample. The amount of protein expression of the target can be increased or reduced when compared with control expression.

A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), Western blot analysis, immunoprecipitation assays, immunofluorescent assays, flow cytometry, immunohistochemistry, HPLC, mass spectrometry, confocal microscopy, enzymatic assays, surface plasmon resonance and PAGE-SDS.

In one embodiment, a method of determining the efficacy of a PAWR inhibitor, e.g., a PAWR inhibitor described herein, is provided. In one embodiment, the method comprises measuring the level of PAWR protein in hPSCs prior to contacting the cells with the PAWR inhibitor and comparing the level of PAWR protein in hPSCs contacted with the PAWR inhibitor with the level of PAWR protein in hPSCs that were not contacted with the PAWR inhibitor.

### Kits

Kits for assessing the activity of an inhibitor of PAWR, e.g., a PAWR inhibitor disclosed herein, are provided. For example, a kit comprising nucleic acid primers for PCR or can be used for assessing PAWR inhibitor efficacy. In another example, a skit comprising an antibody that can detect PAWR can be used for assessing PAWR inhibitor efficacy.

In some embodiments, the kits disclosed herein can be used to predict and assess dissociation-induced death in hPSCs contacted with a PAWR inhibitor, e.g., a PAWR inhibitor disclosed herein. In other embodiments, kits disclosed herein can be used in any of the methods of modifying hPSCs to express a target protein, e.g., as described herein. In one embodiment, a kit disclosed herein comprises instructions for using said kit.

### EXAMPLES

### Example 1

### Genome-Scale CRISPR Screening Identified Novel Human Pluripotent Gene Networks

### Introduction

Human pluripotent stem cells (hPSCs) can be used to generate a wide variety of disease relevant cell-types and have the potential to improve the translation of preclinical research by enhancing disease models. Despite the huge potential, genetic screening using hPSCs has been limited by their expensive and tedious cell culture requirements (Chen et al., 2011), and reduced genetic manipulation efficiencies (Ihry et al., 2017). Only a few shRNAs screens have been conducted in hPSCs (Chia et al., 2010; Zhang et al., 2013), however shRNAs have a high level of off targets and do not cause a complete loss of function, which is difficult to interpret (DasGupta et al., 2005; Echeverri et al., 2006; Kampmann et al., 2015; McDonald et al., 2017). Currently, the CRISPR/Cas9 system is the genetic screening tool of choice because it can efficiently cause loss of function alleles (Cong et al., 2013; Jinek et al., 2012; Mali et al., 2013). Hundreds of genome-scale pooled CRISPR screens have been performed in immortalized human cell lines (Hart et al., 2015; Meyers et al., 2017; Wang et al., 2015). However, in hPSCs the CRISPR/Cas9 system has been primarily used for small-scale genome engineering (Merkle et al., 2015). In hPSCs the only genome-scale CRISPR screen to-date used methods developed for cancer cells, suffered from technical issues, had poor performance, and identified few developmentally relevant genes (Hart et al., 2014; Shalem et al., 2014). We addressed these technical issues by systematically tailoring the CRISPR/Cas9 system for hPSCs (Ihry et al., 2017). A doxycycline inducible Cas9 (iCas9) hPSC line was developed and stably infected with a genome-scale sgRNA library. The CRISPR infected hPSC library was banked and expanded in the absence of editing (−dox), which enabled the generation of a renewable stem cell pool with stable but inactive sgRNAs. This allowed for multiple independent screens to be conducted with the same cell-library.

In the first screen, genes that suppress or enhance hPSC fitness over long-term culture were identified. While previous screens have generated gold standard gene lists of core essential genes that reduce cell survival when mutated, little is known about the mutations that enhance survival and proliferation. Unlike core essential genes, these enhancing mutations appear to be cell type specific and no consistent lists exist for this type of gene (Hart et al., 2014). In hPSCs, karyotypic analysis has detected recurrent copy number variations (CNVs) that confer a growth advantage (Amps et al., 2011; Laurent et al., 2011); however, these studies lack gene level resolution. Recently, next generation sequencing of hundreds of hPSCs identified the recurrence of dominant negative TP53 mutations that can expand within a population of hPSCs (Merkle et al., 2017). Data obtained in this study was mined for gene knockouts that enriched in culture and many genes were identified, including components of the TP53 pathway and other known tumor suppressors. The strongest hit, PMAIP1/NOXA, which, e.g., appears to be a stem cell-specific gene conferring sensitivity to DNA damage downstream of TP53 was validated.

In the second screen, genes required for single cell cloning were identified. hPSCs have a poor survival rate after dissociation to single cells, which is detrimental for genome engineering. Multiple groups have extensively characterized death induced by single cell cloning and have demonstrated the process is similar to, but distinct from anoikis and is triggered by a ROCK/MYOSIN/ACTIN pathway (Chen et al., 2010; Ohgushi et al., 2010). To prevent death, hPSCs are passaged as clumps or treated with ROCK inhibitors (Watanabe et al., 2007). By subjecting our hPSC mutant library to single cell dissociation without ROCK inhibitors, mutations that survive single cell cloning were selected for. sgRNAs for the ROCK and myosin pathways were enriched in the surviving clones. The most enriched gene was the pro-apoptotic regulator PAWR (Burikhanov et al., 2009). Validation studies confirmed a novel role for PAWR as a component of the actin-cytoskeleton that induces membrane blebbing and cell death caused by single cell cloning. The additional novel genes identified here may further our understanding about the sensitivity of hPSCs to single cell cloning.

In the final screen, a FACS-based OCT4 assay was utilized to identify regulators of pluripotency and differentiation. Pluripotency is a defining feature of hPSCs and it allows hPSCs to differentiate into all three germ layers. OCT4/POU5F1, NANOG and SOX2 are critical transcription factors that maintain pluripotency in vivo and in vitro (Chambers et al., 2007; Masui et al., 2007; Nichols et al., 1998). OCT4 and SOX2 overexpression is commonly used to reprogram somatic cells towards the pluripotent state (Takahashi and Yamanaka, 2006; Takahashi et al., 2007; Yu et al., 2007). By isolating mutant cells with high or low OCT4 protein expression, many of the known genes involved in maintaining the pluripotent state along with genes involved with induction of differentiation were identified. The complete list of known and novel genes has been made publicly available to increase the understanding of pluripotency and differentiation.

By using a doxycycline inducible Cas9 (iCas9) hPSC line stably infected with a genome-scale lentiCRISPR library, we banked a CRISPR-hPSC library that was renewable and enabled a high number of independent screens to be performed with the same starting library. This allowed direct comparison between screens and reduced screen to screen variability. The system was rigorously tested and genes important for fitness, pluripotency, and single cell cloning of hPSCs were identified. This example discloses a resource with methods and data which is publicly available including, e.g., many novel genes that are involved in hPSC biology. This resource can serve as a parts list of genes that are, e.g., functionally important for the human stem cell state. Furthermore, the gene sets and methods disclosed herein may, e.g., increase the systematic knowledge of human pluripotent stem cell biology and may enable additional large-scale CRISPR screens in stem cells and their somatic derivatives.

### Materials and Methods

Cell lines
H1-hESCs (WA01-NIHhESC-10-0043)
H9-hESCs (NIHhESC-10-0062)
8402-iPSCs
hDFN-iPSCs
THP-1 cells stably expressing Cas9 were cultured and mutagenized as described by Feuerbach et. al., 2017. Karyotyping was performed by Cell Line Genetics (Madison, Wis.).

### Genome Engineering

Dox treated H1-iCas9 cells were subjected to three successive rounds of RNAimax delivery of the two-component synthetic crRNA/tracrRNA (IDT) pairs targeting PMAIP1, PAWR and TP53 as described by Ihry et al., 2017. PMAIP1, and PAWR were transfected with a single sgRNA while TP53 was co-transfected with 3 crRNAs. CRISPR indel analysis detect complete gene disruption for all three genes and was performed as described by Ihry et. al., 2017.

PMAIP1 crRNA 3
TCGAGTGTGCTACTCAACTC
PAWR crRNA 5
CGAGCTCAACAACAACCTCC
TP53 crRNA 1
GAAGGGACAGAAGATGACAG
TP53 crRNA 2
GAAGGGACAGAAGATGACAG
TP53 crRNA 4
GAGCGCTGCTCAGATAGCGA
P21 crRNA 1
AATGGCGGGCTGCATCCAGG
P21 crRNA 4
TCCACTGGGCCGAAGAGGCGG
P21 crRNA 6
GGCGCCATGTCAGAACCGGC

### Pooled Mutagenesis (CRISPR Nuclease/Interference)

H1-hESCs expressing a constitutive (c) Cas9-KRAB knocked-in to the AAVS1 locus were generated as described by Ihry et., al., 2017. The following lentiCRISPR were used to transduce iCas9 or cCas9-KRAB cells to generate mutant cell pools.

### CRISPR Nuclease

PAWR sgRNA 1
TTTGGGAATATGGCGACCGG
PAWR sgRNA 2
GGTGGCTACCGGACCAGCAG
PAWR sgRNA 5
CGAGCTCAACAACAACCTCC
MAPT sgRNA 1
GAAGTGATGGAAGATCACGC
ACSL4 sgRNA
TGGTAGTGGACTCACTGCAC
ARSH sgRNA
GCAGCACCGTGGCTACCGCA
BMX sgRNA
ATGAAGAGAGCCGAAGTCAG
BTK sgRNA
GGAATCTGTCTTTCTGGAGG
GABRA3 sgRNA
AAGGACTGACCTCCAAGCCC
GK sgRNA
TAGAAAGCTGGGGCCTTGGA
NRK sgRNA
CGCCTTCCTATTTCAGGTAA
TLR7 sgRNA
CAGTCTGTGAAAGGACGCTG

### CRISPR Interference

PAWR sgRNA 1
GGCGCGCTCGAGGACTCCAA
PAWR sgRNA 2
GTTGCAGGGTGGGGACCCGG
PAWR sgRNA 3
GCTGGCCGGTAGTGACTGGT
PAWR sgRNA 5
GGCTGCTGGCCGGTAGTGAC

Large-Scale Culture of hPSC

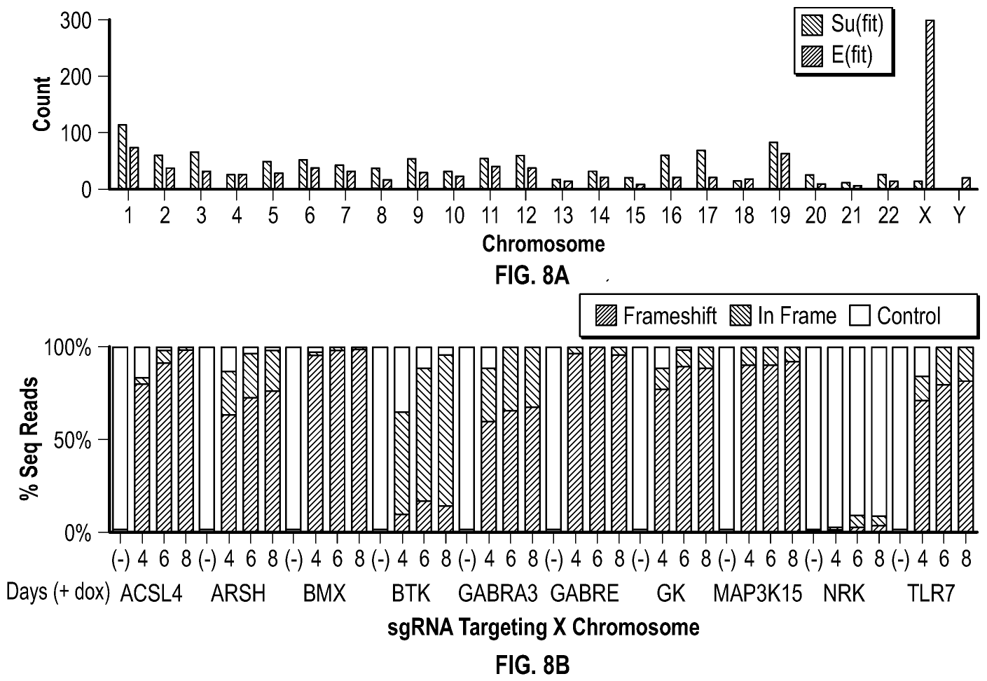

H1-hESCs with AAVS1 knock in of the iCas9 transgene were generated and cultured in TeSR-E8 media (STEMCELL TECH.-05940) on vitronectin (Gibco-A14700) coated plates as described by Ihry et. al., 2017. The large-scale culture of hPSCs is not routine. Pilot studies with a sub-genome sgRNA library used a total of 40 individual T225 flasks and was cumbersome (Ihry et. al., 2017). Daily feeding and passaging in which multiple flask needed to be pooled was time consuming and increased the risk for contamination. To minimize the manipulation required during feeding and passaging we used 5-layer CellSTACKs. The vessels were large enough to contain an entire 55,000 sgRNA at roughly 1000× coverage per sgRNA at a seeding density of 21,000 cells/cm² (Seed 66*10\hat{ }6 cells for ˜1200×). Only 4 to 8 5-layer CellSTACKs were growing at a given time during the month-long genome-scale CRISPR screen. Given the expense of E8 media, pen/strep was added for the first screen at scale. After running a lengthy genome-scale screen and becoming experienced with large-scale hPSC culture, future screens were performed without pen/strep (FIGS. 8A-8B).

lentiCRISPR Packaging

For a one layer CellSTACK 42 million HEK293T (66,000 cells/cm²) were plated in 100 mL of media (DMEM+10% FBS+1× NEAA, no pen/strep). One day after seeding, cells were transfected with single lentiCRISPR plasmids in 6-well plates or pooled lentiCRISPR plasmids in CellSTACKs. For a one layer CellSTACK 102 uL of room temp TransIT (Mirus MIR 2700) and 3680 uL Opti-MEM (Invitrogen 11058021) were mixed incubated in a glass bottle for 5 minutes at room temp. 94.5 ug of Lentiviral Packaging Plasmid Mix (Cellecta CPCP-K2A) and 75.6 ug of the lentiCRISPR plasmid library was added to the transfection mix and incubated for 15 minutes at room temp. After incubation, the mix was added to 100 mL of fresh media and the cells were fed. The next day the transfected cells received 100 ml fresh media. After 3 days of viral production supernatants were filtered (.45uM corning 430516) and aliquoted in to 1 ml tubes for storage at −80 C.

Large-Scale Transduction of hPSC

A genome-scale CRISPR screen was conducted using a 110K sgRNA library (˜5 sgRNAs per gene, split into two 55K sgRNA sub-pools, DeJesus et al., 2016). A 1000× coverage of each sgRNA was sought to offset cell loss from double strand break (DSB)-induced toxicity (Ihry et. al., 2017). To screen a sufficient number of cells, hPSCs were infected with lentiCRISPRs in 5-layer CellSTACKs. Cells were infected at .5 MOI to ensure each cell was infected with no more than a single sgRNA. After puromycin selection cells were expanded for one week without dox to be pelleted for DNA, banked or screened.

Pooled CRISPR screens rely on cells being efficiently transduced at less than or equal to 0.5 MOI. We developed a reverse transfection method for hPSCs without polybrene that resulted in an efficient transduction with low volume exposure to HEK293T lentiviral supernatants. LentiCRISPR plasmids expressed a constitutive RFP and puromycin resistance to mark and select for infected cells respectively. Viral titer of the two 55,000 sgRNA libraries expressing RFP in 6-well plates determined that less than 25 uL in 1.5 mL of media was required for 0.5 MOI. These calculations scaled appropriately in 5-layer cell stacks with 500 mL of media and approximately 50% of the cells were RFP positive in the absence of puromycin.

A genome-scale lentiCRISPR library targeting each gene 5 times has been split into 55,000 sub-pools (pool 1 and 2). To screen at 1000× per sgRNA, 264 million hPSCs are infected in 4×5-layer CellSTACKS (12,720 cm2, 21,000 cells/cm2). Cells are infected at 0.5 MOI to ensure only one sgRNA is expressed per cell (+sgRNA/puroR/RFP). After puromycin selection cells are expanded until confluent (4-6 days). At this point 40 million cells (10 million/ml) can be banked in 5 ml cryovials for use at a later date.

Banking LentiCRISPR Infected hPSC Library

One 5-layer cell stack was treated with 200 mL accutase that was evenly distributed among layers. After incubation at 37 C for 10 minutes, accutase (Gibco-A1110501) was neutralized with 200 mL E8 media. Cells were counted and pelleted to be resuspended at a concentration of 10*106 million cells per ml in a solution of 40% Tet-free FBS (Seradigm #1500-500) and 10% DMSO (Sigma D2650) and 50% E8 media. 4 ml aliquots were placed in 5 ml cryovials and frozen in a Mr. Frosty (Thermo Scientific 5100-0001) at −80 C overnight before long-term storage in liquid nitrogen. Thawing of cells banked in 5 ml cryovials showed an average viability around 85% for both lentiCRISPR pools. Viability was assayed using a Nexcelom Cellometer Auto 2000 and AO/PI (Nexcelom CS2-0106) staining solution.

The effect of freezing and thawing was tested on the representation of the sgRNAs in the library by thawing the cell library at either 700× (40 million cells per 55 k sub-pool) or 2100× (120 million cell per 55 k sub-pool) cells per sgRNA (FIG. 8B). Cells were thawed in a 37° C. water bath and transferred to a 50 mL conical with E8 media and centrifuged at 300 g for 3 minutes. Pelleted cells were resuspended in E8 media and replated at a density of 30 to 40*10⁶ cells/cm². 1 cryovial with 40 million cells was thawed and plated on a 2-layer cell stack (40/55 million=700×). 3 cryovials with 120 million cells was thawed and plated on a 5-layer cell stack (120/55 million=2100×).

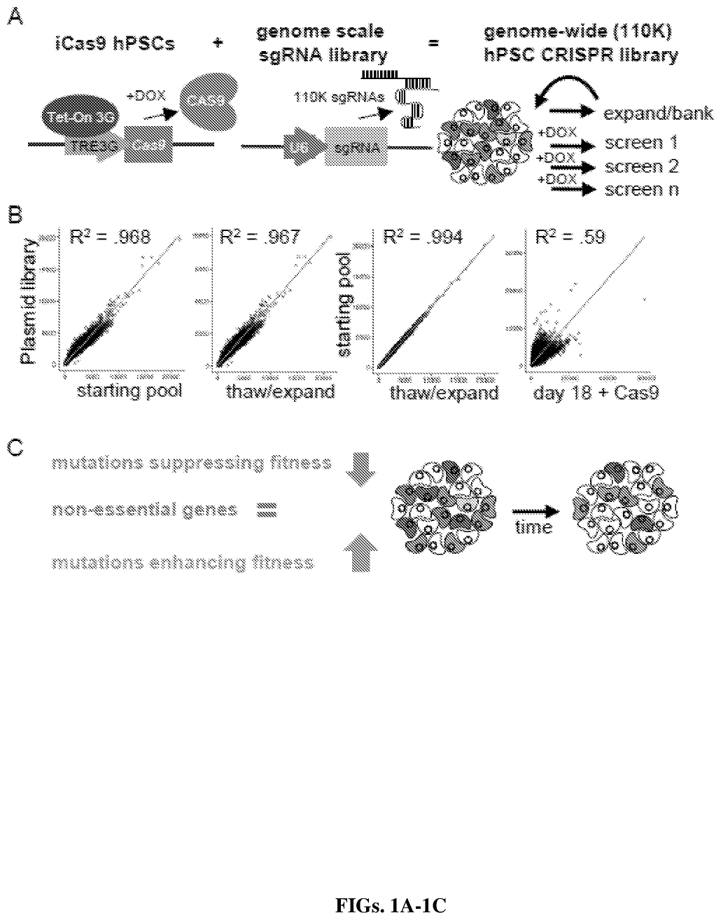

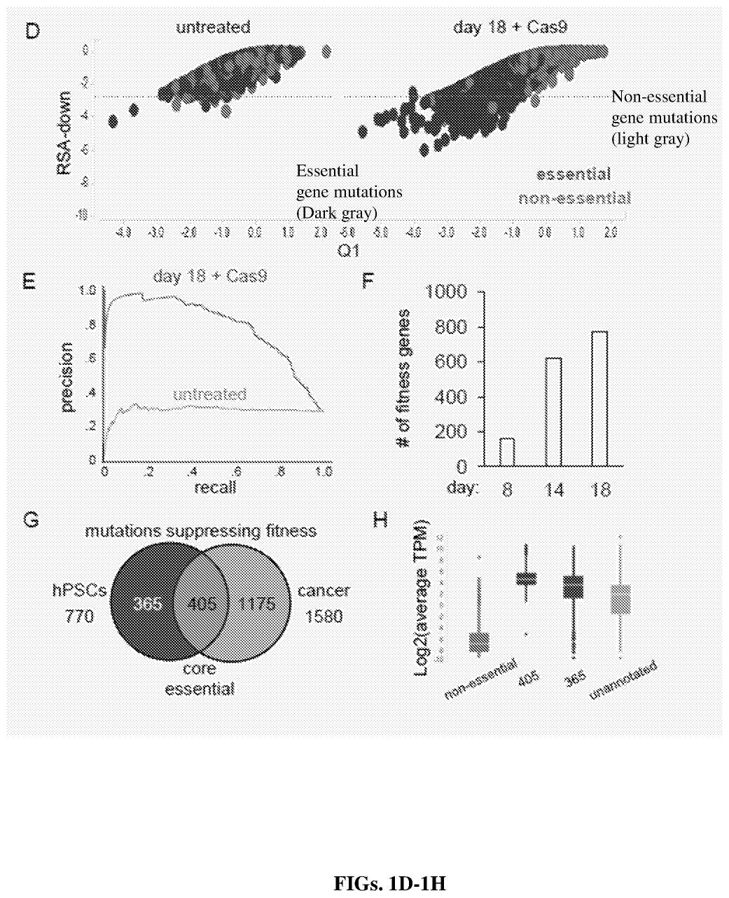

After thawing and feeding the cells for two days, DNA was isolated and analyzed by NGS to measure the representation of each sgRNA in the pool. Both day zero and freeze/thaw samples had an over 87% alignment of sequencing reads and fewer than 25 missing barcodes per replicate. Pearson correlation analysis allowed us to demonstrate that there was a high correlation between the day zero samples and the freeze/thaw samples. Calculating normalized sgRNA counts revealed a strong correlation between the cell library before (day 0) or after one freeze/thaw cycle (FIG. 1B). The iCas9 system allowed us to renormalize to the starting cell library, which further increased the correlation between the day cell library and thawed cell library (FIG. 1B). Subsequent exposure to Cas9 caused a subset of sgRNAs to enrich or dropout of the population over time and decreased the correlation (FIG. 1B). Cumulatively, this demonstrated that it is possible to expand and bank a large lentiCRISPR hPSC library that can be utilized for successive rounds of screening.

### OCT4 FACS-BASED Screen

Cells were dissociated using accutase for 10 min at 37 C to create a single cell suspension which strained using a 40-micron filter and was counted. After removing accutase, pelleted cells were resuspended in a volume of 1 million cells/mL for the staining protocol. For each replicate 55 million unsorted cells were frozen down prior to fixation. The remaining cells were fixed in 4% PFA in PBS for 10 minutes at room temperature on a rocker. Cells were spun down at 300 RCF for 3 min between each subsequent solution change. Cells were washed with 0.1% Triton-X in PBS after fixation and blocked in 2% goat serum, 0.01% BSA and 0.1% triton X in PBS for 1 hr at room temperature. Conjugated (AF488) primary antibodies specific to OCT4 (CST 5177) were diluted in blocking solution (1:200) and incubated with cells on a rocker over night at 4 C. Prior to FACS analysis cells were resuspended in PBS at a concentration of 30 million cells/mL. A total of 1.2 billion cells were sorted into OCT4low (50 million cells) and OCT4high (61 million cells) populations using an ARIA III (BD).

### DNA Isolation

For each replicate, 55 million cells (1000×) were pelleted and genomic DNA was isolated using QIAamp DNA Blood Maxi Kit (Qiagen 51194) as directed by manufacturer. Isolating genomic DNA from 4% PFA fixed cells was performed by utilizing phenol chloroform extraction. Cells were resuspended in 500 ul TNES (10 mM Tris-Cl ph 8.0, 100 mM NaCL, 1 mM EDTA, 1% SDS) and incubated overnight at 65° C. After allowing the samples to cool, 10 ul of RNase A (Qiagen 19101) and samples were incubated at 37° C. for 30 minutes. Next, 10 ul of proteinase K was added (Qiagen 19133) and incubated for 1 hour at 45° C. Following this, 500 ul of PCIA (Phenol;Cholorform;Isoamyl alcohol ph8) (Thermo 17908) was added and the samples were vortexed. Samples were then spun in a centrifuge at max speed for 2 minutes. The aqueous phase was transferred to 500 ul of PCIA and vortexed followed by a spin at max speed for 2 minutes. The aqueous phase was then transferred to 450 ul of chloroform and vortexed followed by a spin at max speed for 2 minutes. The aqueous phase was transferred to 40 ul of 3M NaAcO ph 5.2. 1 ml of 100% ethanol was added, followed by mixing and precipitation of DNA for 1 hour on ice. The samples were spun at max speed for 2 minutes, and then decanted and washed with 1 ml 70% ethanol, followed by a spin at max speed for 1 minutes. Finally, the samples were decanted and the pellet was air dried. The pellet was resuspended in 50 ul of nuclease free H20.

mRNA Expression

qPCR and RNA-seq analysis were performed as described by Ihry et. al., 2017. RNA-seq data for 14 iPSC control lines restricted to the expression for PAWR and PMAIP1 will be made available upon request.

Assaying Sensitivity to DNA Damage

Control and mutant iCas9 H1-hESCs expressing a MAPT targeting sgRNA were monitored daily post-media change using an IncuCyte zoom (Essen Biosciences). At the onset of the experiment cells were plated at density of 10,500 to 21,000 cells/cm² and cultured plus or minus dox for the duration. Confluence was calculated using the processing analysis tool (IncuCyte Zoom Software).

Assaying Survival without ROCK Inhibitor

Control and mutant iCas9 H1-hESCs were dissociated with accutase for 10 minutes. Flowmi 40-micron cell strainers (BEL-ART H13680-0040) were used to ensure a uniform single cell suspension prior to replating cells. Cells were plated at a density of 10,500 to 21,000 cells/cm² plus or minus thiazovivin (ROCK inhibitor). Timelapse images were taken in 1 hr intervals using IncuCyte zoom (Essen Biosciences). The confluence processing analysis tool (IncuCyte Zoom Software) calculated confluency for each sample.

### Immunofluorescence and Western Blotting

Immunofluorescence staining of fixed cells was performed as described by Ihry et. al., 2017. Protein lysates were made by vortexing cell pellets in RIM buffer (Thermo Scientific 89901) supplemented with Halt protease inhibitor cocktail (Thermo Scientific 78430) and phosphatase inhibitors (Thermo Scientific 1862495). Samples were incubated at 4 C for 10 minutes before centrifugation at 14,000×g for 10 minutes at 4 C. Supernatants were transferred to new tubes and quantified using the BCA protein assay kit (Thermo Scientific 23225) and a SpectraMax Paradigm (Molecular Devices) plate reader. Samples were prepared with NuPAGE LDS sample buffer 4× (Invitrogen NP0008) and NuPAGE sample reducing agent (Invitrogen NP0009) and heated for 10 minutes at 70 C. Chameleon Duo Pre-stained Protein Ladder (LiCOR P/N 928-60000) was loaded alongside 10 ug of protein per sample on a NuPAGE 4-12% Bis-Tris Protein Gels, 1.5 mm, 10-well (Invitrogen NP0335BOX). Gel electrophoresis was performed at 150 V for 1 hr in NuPAGE MOPS SDS Running buffer (20×) (Invitrogen NP0001) using a XCell SureLock Mini-Cell (Thermo Scientific E10002). Transfer was performed using an iBlot 2 dry blotting system (Thermo IB21002 and IB23002) as described by manufacturer. Blots were blocked in TBS blocking buffer (LiCOR 927-50000) for 1 hour at room temperature. The blots were then incubated with primary antibodies diluted in TBS over night at 4 C. Blots were washed 3× in PBST and incubated with secondary antibodies diluted in TBS for 2 hours at room temperature. Blots were imaged using an Odyssey CLx (LiCOR).

### Primary Antibodies

Phallodin-647 (ThermoFisher Scientific A22287)—1:40
SCRIB (Abcam ab36708)—1:100 (IF)
PRKCZ (Abcam ab59364)—1:100 (IF)
PAWR/PAR-4 (CST-2328)—1:100 (IF) 1:1000 (WB)
Cleaved Caspase-3 Asp175 (CST-9661)—1:200 (IF) 1:1000 (WB)
GAPDH (Enzo ADI-CSA-335-E)—1:1000 (WB)
aTUB (Sigma 76199)—1:10000 (WB)

### Secondary Antibodies

IRDye 800CW anti-rabbit (LiCOR 926-32211)—1:5000
IRDye 680RD anti-mouse (LiCOR 926-68070)—1:5000
AF488 conjugate Goat anti-Rabbit IgG (H+L) (ThermoFisher-A-11008)—1:500

### Results

iCas9 System is a Self-Renewing Resource Enabling Successive Genome-Wide Genetic Screens in hPSCs

A high-throughput CRISPR/Cas9 platform for hPSCs was developed which enabled successive rounds of screening from a stable library of lentiCRISPR infected hPSCs (FIG. 1A). Generating a genome-scale lentiCRISPR hPSC library enabled both the rigorous testing of CRISPR screen performance and the identification of cell type-specific regulators of the pluripotent state. In our previous work, we developed an all-in-one doxycycline (dox) inducible Cas9 (iCas9) transgene that was inactive in the absence of dox (Ihry et al., 2017). The tight control over Cas9 expression allowed us to transduce cells with lentiviruses expressing sgRNAs (lentiCRISPRs) in the absence of dox without causing on-target indels. We tested if it was possible to bank a genome-scale lentiCRISPR infected cell library (5 sgRNAs per gene, 110,000 total sgRNAs) prior to Cas9 mutagenesis (−dox). After one freeze-thaw cycle, NGS analysis revealed no bottlenecking of the library demonstrating the feasibility of banking a large lentiCRISPR hPSC library for repeated screens (FIG. 1B).

Evaluating the Performance of CRISPR Screening in iCas9 hPSCs

Next, a fitness screen was performed to evaluate the global performance of the system (FIG. 1C). The performance of the screen was benchmarked by utilizing annotated lists of core essential genes. Core essential genes are required for the survival of all cells and the corresponding CRISPR knockout causes the sgRNAs to be depleted (Hart et al., 2014, 2015). Genome-scale CRISPR screening in hPSCs has been challenging (Hart et al., 2014; Shalem et al., 2014). hPSCs have a strong DNA damage response (DDR) and Cas9-induced double strand breaks (DSBs) cause a significant cell loss (Ihry et al., 2017). Failure to account for Cas9-induced cell loss is problematic for pooled screening because it is critical to maintain representation of each sgRNA barcoded cell. Our previous work demonstrated a range of Cas9-induced cells loss between 3 to 10-fold across many sgRNAs (Ihry et al., 2017). To prevent bottlenecking of the sgRNA library the screen was conducted in hPSCs at an average of 1000 cells per sgRNA (total of 110 million infected cells). By doing this about 4-fold more cells were maintained compared to a typical cancer screen (Hart et al., 2015). During the fitness screen, DNA was sampled before and after dox exposure at days 0, 8, 14, and 18. To provide a qualitative measurement of screen performance the p-values calculated by the redundant siRNA activity (RSA) test was plotted against Q1 based z-scores for a set of core essential and non-essential genes (Hart et al., 2014; Konig et al., 2007). Before dox treatment the non-essential and core essential genes were randomly distributed within a tight cluster (FIG. 1D). After 18 days of Cas9 treatment the distribution spread and the essential genes significantly dropped-out while the non-essential genes remained constant (FIG. 1D, and Table 1-2.

Next, the Bayesian Analysis of Gene EssentiaLity (BAGEL) algorithm which calculates a Bayes factor for each gene by determining the probability that the observed fold change for a given gene is an essential gene was employed to quantify performance (Hart and Moffat, 2016).This generated a ranked list of Bayes factors for each gene, which was then used to quantify screen performance by precision versus recall analysis. In a high-performance screen, essential genes have high Bayes factor scores and the precision versus recall curve gradually drops off as analysis of the ranked list is completed. In contrast, a poor performing screen has a precision versus recall curve that rapidly drops off, indicating many false positives (non-essential genes) with high Bayes factor scores. The sample without dox exposure (untreated) had a randomly ranked Bayes factor list with non-essential and essential genes interspersed and exhibited a poor precision versus recall curve (FIG. 1E, and Table 3. In the day 18 Cas9 (+dox) treated samples, essential genes and non-essential genes segregated from each other and generated a high performing precision versus recall curve that gradually dropped off (FIG. 1E, F and Table 3).

After 18 days of Cas9 exposure 770 fitness genes were identified at a 5% false discovery rate based off of the precision calculation (FIG. 1F, and Table 4. A comparison of the set of 770 hPSC fitness genes to 1580 core essential genes from cancer lines revealed an overlap of 405 genes (FIG. 1G) (Hart et al., 2015). The remaining 365 specifically dropped out in hPSCs. Both the core and hPSC-specific essential genes were abundantly expressed in hPSCs and further supported the observation that they are required for hPSCs culture (FIG. 1H). The fitness screen in hPSCs correctly identified the dropout of core-essential genes with accuracy that is on par with CRISPR screens conducted in cancer cell lines. This demonstrated that cancer cells and stem cells share a common set of core essential genes that can be used to benchmark performance. By properly accounting for cell loss caused by Cas9 activity, a significant technical barrier was overcome that has thwarted previous attempts at genome-scale screening in hPSCs (Hart et al., 2014; Shalem et al., 2014). This demonstrated that it is possible to conduct a genome-scale CRISPR screen in hPSCs using the methods described herein.

TP53 Pathway Mutations Specifically Enrich During CRISPR Fitness Screen in hPSCs

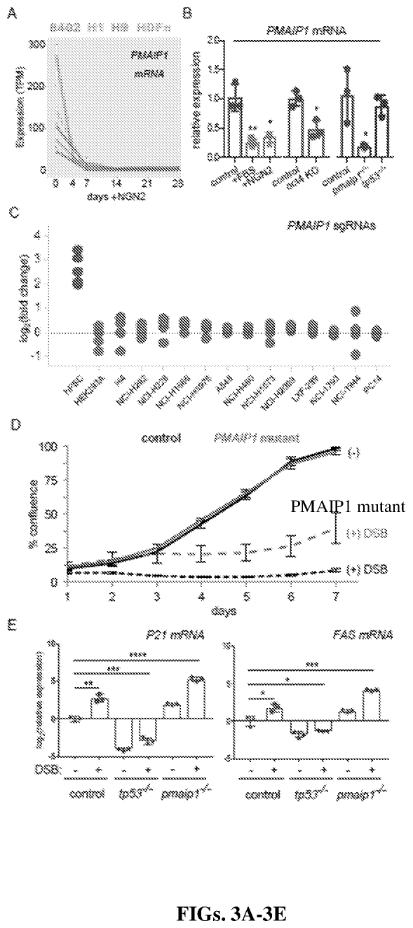

Curated list of genes that enhance fitness during a CRISPR screen do not exist, making it difficult to benchmark the enrichment results (Hart et. al., 2015). By comparing the top ˜1000 depleted (RSA-down<−2.25, Table 2) and enriched (RSA-up<−2.25, Table 5) genes, it was observed that 34.5% (318 of 922) of the enriched genes were located on the X and Y chromosomes (H1-hESCs XY, Table 2). In contrast, the depleted genes were evenly distributed across all chromosomes. The data showed that allosome targeting sgRNAs behaved similarly to non-targeting controls which enriched during a CRISPR screen in hPSCs (Ihry et al., 2017). It was observed that sgRNAs causing a single DSB on X chromosome were less toxic relative to sgRNAs inducing 2 DSBs at the MAPT locus despite being able to efficiently induce indels (FIGS. 8A-8B). Others have observed similar effects in the opposite direction. sgRNAs targeting genomic amplifications in cancer cell lines exhibit a strong depletion irrespective of the gene targets (Aguirre et al., 2016; Meyers et al., 2017; Munoz et al., 2016). Unlike cancer cell lines, H1-hESCs with a normal karyotype are very sensitive to DNA damage making the difference between 1 and 2 DSBs significant. After recognizing that the enrichment of sgRNAs on the X/Y chromosomes was related to DSB sensitivity and copy number differences in male H1-hESCs these were removed from further analysis. In the remaining list of 603 autosomal genes that were enriched (RSA-up −2.25) identified 50 tumor suppressor genes were identified (FIG. 3A, and Table 5) (Zhao et al., 2016).

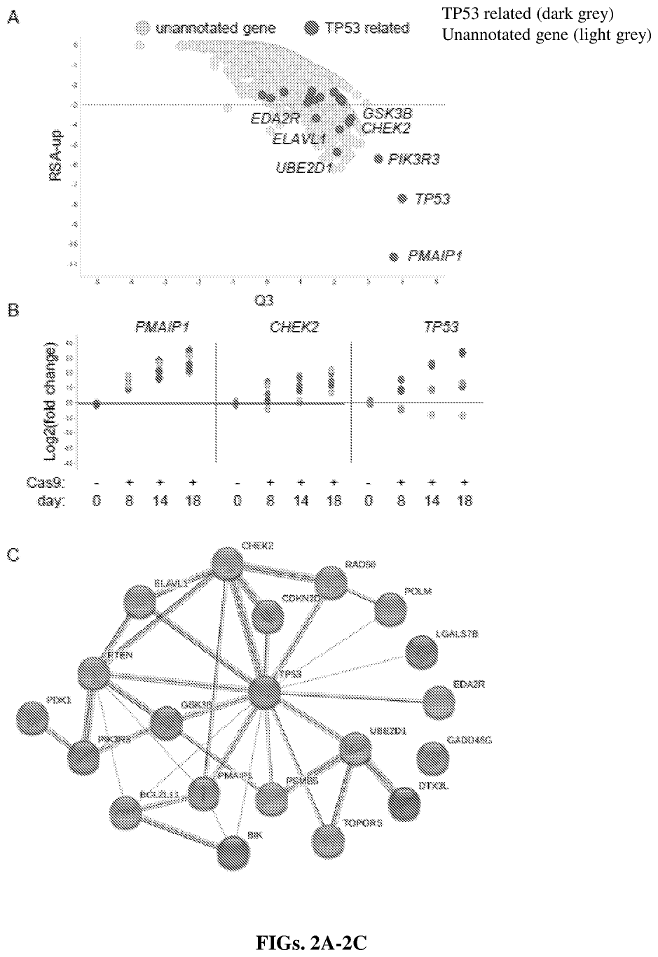

The second most enriched gene was TP53 and confirmed the selective pressure imposed by Cas9-induced DSBs in hPSCs during a CRISPR screen (FIG. 2A). Consistent with this TP53 mutants are able to suppress cell loss induced by Cas9 activity (Ihry et al., 2017). Throughout the 18-day screen, the representation of sgRNAs targeting TP53 (Chr. 17), the checkpoint kinase, CHEK2 (Chr. 22), and the proapoptotic regulator, PMAIP1 (Chr. 18), increased in a time-dependent manner (FIG. 2B). Database mining for associations with TP53 among the enriched genes identified 20 genes with direct connections to TP53 (FIG. 2C, and Table 6). We hypothesized that these genes could include additional regulators responsible for the extreme sensitivity to DNA damage in hPSCs.

PMAIP1 was the most enriched gene in the screen. PMAIP1 has been implicated in TP53-dependent cell death and functions by sensitizing cells to apoptosis by antagonizing the anti-apoptotic, MCL1, at the mitochondria (Kim et al., 2006; Perciavalle et al., 2012; Ploner et al., 2008). PMAIP1 is highly expressed in hPSCs and its expression marks the pluripotent state (Mallon et al., 2013). This observation was confirmed by examining PMAIP1 expression in 2 iPSC and 2 hESC lines. Analysis of RNA-seq experiments confirmed that PMAIP1 highly expressed during the pluripotent state and drops during neuronal differentiation using NGN2 transgene (FIG. 3A). Additionally, GTEx data revealed that PMAIP1 is not expressed in most tissues (GTEx Analysis Release V7). Although it has been demonstrated that PMAIP1 expression is maintained by OCT4 in testicular germ cell tumors (Gutekunst et al., 2013), no functional connection has been made in hPSCs. Next, experiments were performed to test whether PMAIP1 expression was maintained by the pluripotency network by differentiating cells or knocking out OCT4. Under these conditions qPCR detected a significant decrease in PMAIP1 mRNA (FIG. 3B). In thymocytes, PMAIP1 mRNA is induced by TP53 (Khandanpour et al., 2013). TP53 knock out hPSCs were tested, and no reduction in PMAIP1 mRNA was detected (FIG. 3B). hPSCs constitutively express high levels of PMAIP1 and DNA damage does not increase PMAIP1 expression, which further supports PMAIP1 mRNA is pluripotency-dependent and TP53-independent (Thry et al., 2017).

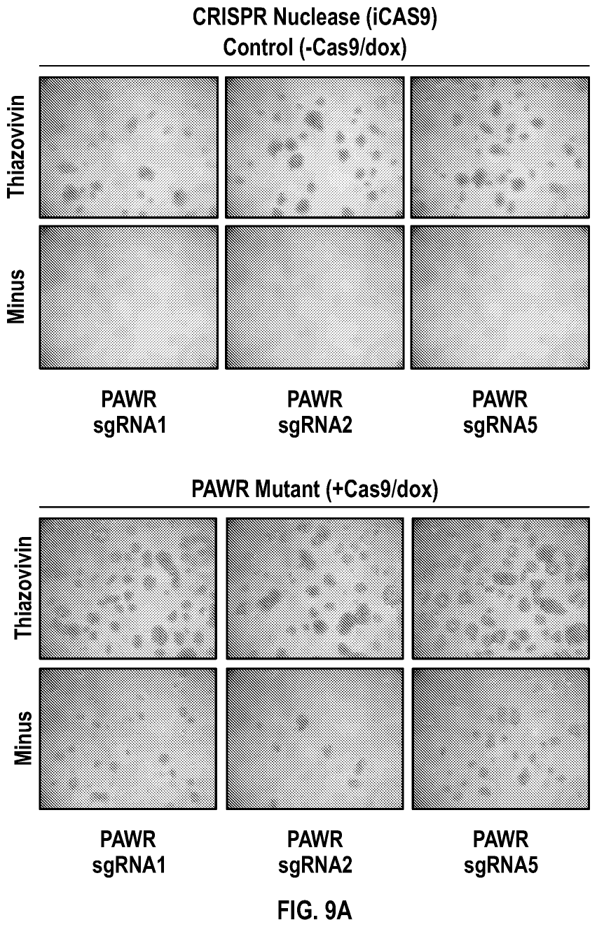

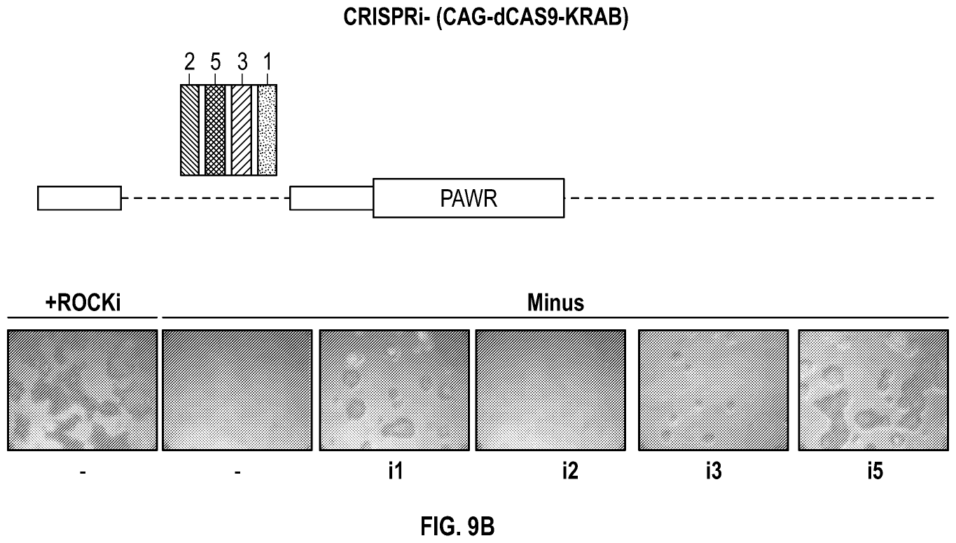

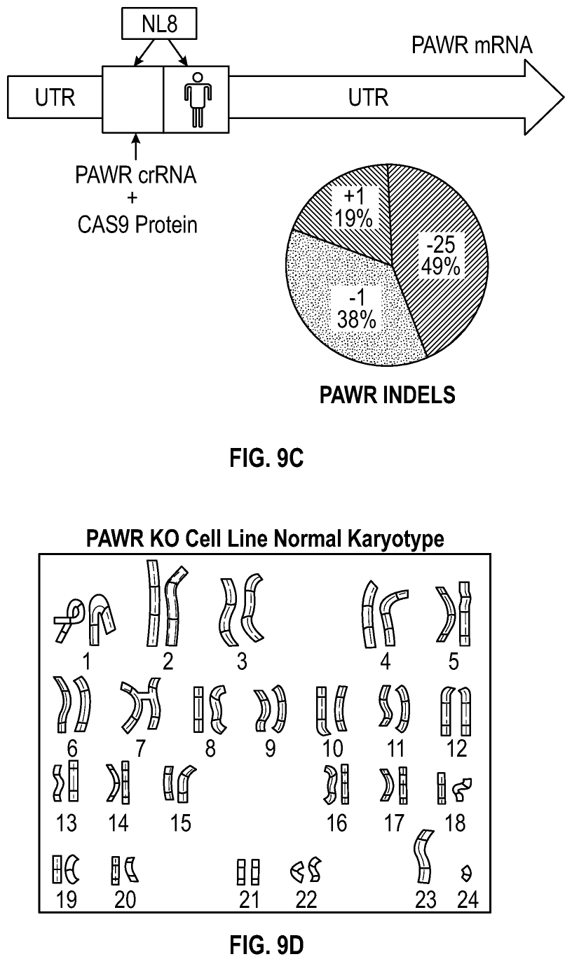

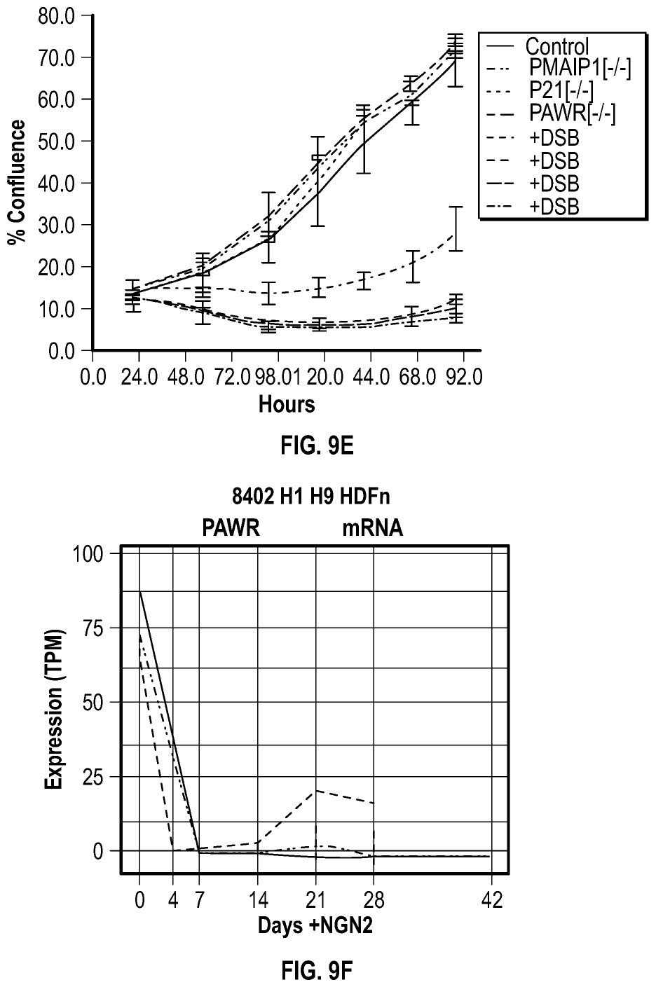

Prior work demonstrated that cancer lines have a reduced DNA damage response relative to hPSCs (Ihry et al., 2017). Consistent with this an enrichment of PMAIP1 sgRNAs was not observed in 14 independent CRISPR screens conducted in cancer cell lines despite using the same sgRNA library (FIG. 3C). This suggested that PMAIP1 is responsible for making hPSCs sensitive to DNA damage. To test the functional consequences of PMAIP1 mutations a knock out iCas9 cell line was made using transient exposure to synthetic crRNAs. Experiments were performed to test whether PMAIP1 mutants were resistant to DSB-induced death by using lentiCRISPRs to deliver a sgRNA targeting MAPT, a neuronal gene not expressed in hPSCs. In the absence of Cas9 (-dox) both control and PMAIP1 mutant iCas9 cells grew at a similar rate while expressing a sgRNA. In the presence of Cas9 (+dox) and a sgRNA, control cells died while PMAIP1 mutants were able to survive despite efficient DSB induction (FIG. 3D, and FIGS. 9A-9F). qPCR analysis of the TP53 target genes P21 and FAS detected an elevated expression in PMAIP1 mutants compared to controls (FIG. 3E). Despite having an active TP53, PMAIP1 mutants survive. This indicated that PMAIP1 is downstream of TP53 activation and is consistent with its known role as a sensitizer to apoptosis (Ploner et al., 2008). In the retinal pigment epithelial cell line RPE-1, Cas9 activity causes TP53-dependent cell cycle arrest and genome-scale screening in RPE-1 cell detected an enrichment of both TP53 and its target P21/CDKN1A a cell cycle regulator (Haapaniemi et al., 2017). In hPSCs cell death is the predominant response to DNA damage. Unlike PMAIP1 mutants, P21 mutants (>80% indels) were unable to suppress DSB-induced toxicity (FIG. 9E). Also, P21 sgRNAs did not enrich in H1-hESCs which exhibited an apoptotic response to DNA damage and likewise PMAIP1 sgRNAs were not enriched in the cell cycle arresting RPE-1 cell screen (Table 5) (Haapaniemi et al., 2017). Overall, these results indicated that PMAIP1 plays a role in the sensitivity of hPSCs to DNA damage and highlights the ability of genome-scale CRISPR screens to identify cell-type specific genes important for maintaining the pluripotent state.

### Genetic Screen for Suppressors of Dissociation-Induced Death

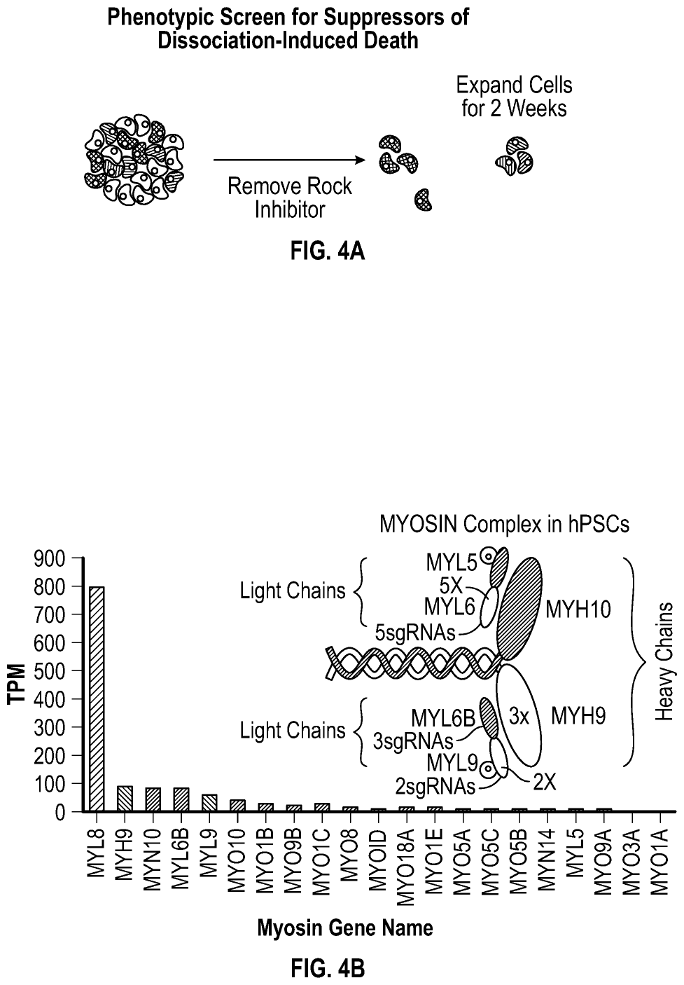

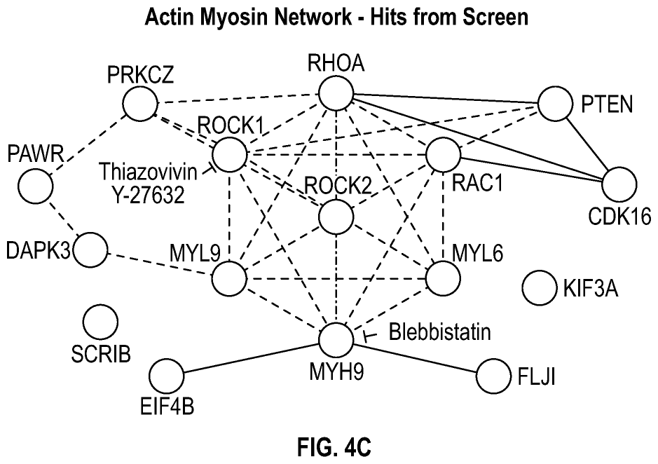

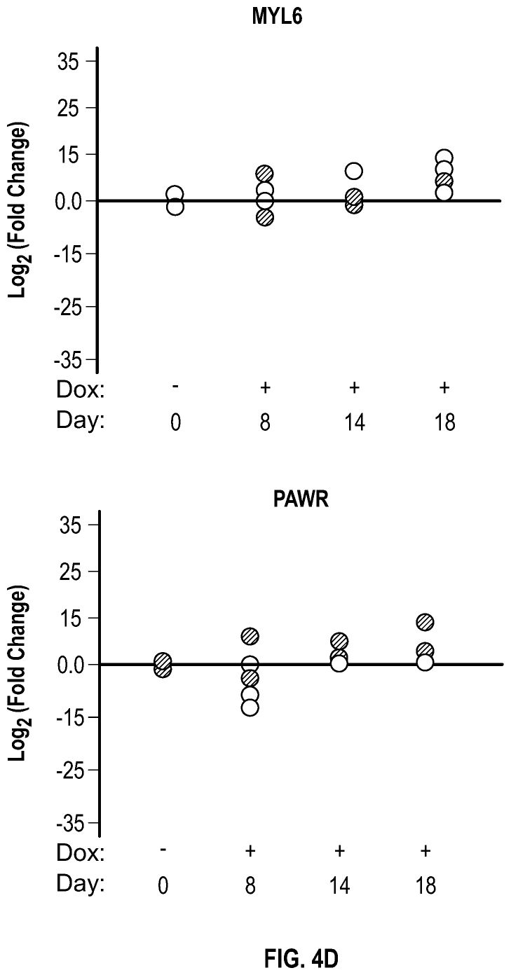

Next, the ability to identify phenotypic regulators of human developmental processes was tested. hPSCs, unlike mESCs, are very sensitive to dissociation and die in the absence of ROCK-inhibitors (Ohgushi et al., 2010). During dissociation of hPSCs, Rho and ROCK become activated. This leads to the phosphorylation of MYOSIN, which causes membrane blebbing and cell death. To promote survival, inhibitors that target ROCK (Y-27632/thiazovivn) or MYOSIN (blebbistatin) are used routinely during hPSC passaging (Chen et al., 2010; Watanabe et al., 2007). In the absence of ROCK inhibitors very few cells survive dissociation. Importantly, this phenotype is developmentally rooted. hPSCs are epiblast-like and cells that fail to incorporate into the polarized epithelium of the epiblast undergo cell death in the embryo (Ohgushi et al., 2010). To gain a deeper understanding of the genes involved, suppressors of dissociation-induced death were screened for. During the day 14 passage of the fitness screen an additional replicate of the genome-scale mutant cell library was plated in the absence of thiazovivin (FIG. 4A).

The majority of the cells died during this process and the surviving cells were maintained for two weeks until large colonies were visible. DNA was isolated, analyzed by NGS and identified 76 genes with 2 or more independent sgRNAs surviving dissociation without thiazovivin (FIG. 4C and Table 7). As expected multiple sgRNAs targeting ROCK1 and MYH9, the genetic targets of ROCK inhibitors and blebbistatin, were recovered. Myosin is a hexameric motor protein that is comprised of 6 subunits with 3 subtypes. The screen recovered 3 sgRNAs for MYH9 a myosin heavy chain, 5 sgRNAs for MYL6 a non-phosphorylated myosin light chain, and 2 sgRNAs for the ROCK target MYL9/MLC2 a phosphorylated myosin light chain. There are many myosin proteins and the screen described herein identified 3 out of 5 of the most abundantly expressed hPSCs (FIG. 4B). This data supports the importance of myosin activation in membrane blebbing and dissociation-induced death. A number of additional genes, e.g., with roles in the actin/myosin network or cytoskeleton, including DAPK3, PAWR, OPHN1, FLII and KIF3A were identified (FIG. 4D). Overall STRING-DB analysis detected a connected set of genes with ties to the actin/myosin regulatory network (FIG. 4D). In general members of this network did not enrich during the fitness screen suggesting, e.g., that they specifically regulate dissociation-induced death and not fitness (FIG. 4E).

### PAWR is Required for Dissociation-Induced Death

For follow-up studies, experiments were performed focusing on the strongest hit from the screen, PAWR a pro-apoptotic regulator (Hebbar et al., 2012). PAWR has no known biological role in the early embryo or during dissociation-induced death of hPSCs. The screen recovered all 5 sgRNAs targeting PAWR in the genome-scale library and the barcode reads were highly abundant (FIG. 4C). Unlike sgRNAs for TP53 pathway that enriched throughout the CRISPR screen, PAWR and MYL6 had no effect on fitness in the presence of thiazovivin (FIG. 4E). We repeated the results using 3 independent lentiCRISPRs to knock out PAWR in iCas9 expressing H1-hESC cells. In the absence of thiazovivin, control cells did not survive (FIGS. 9A-9F). In contrast, PAWR mutants were able to survive without thiazovivin (FIGS. 9A-9F). Independently, CRISPRi sgRNAs targeting PAWR also promoted survival in the absence of thiazovivin (FIGS. 9A-9F).

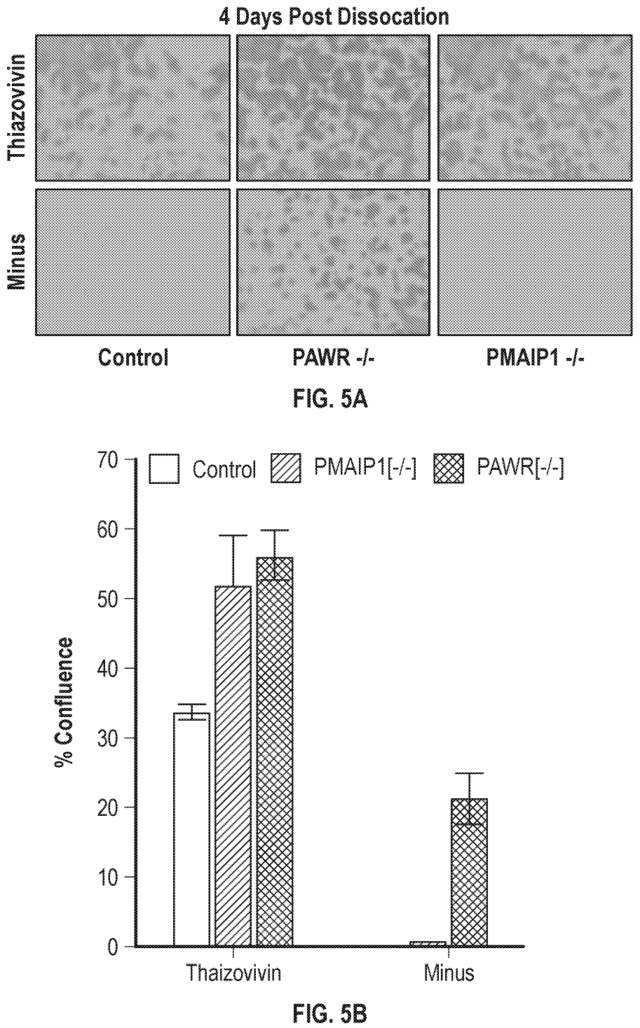

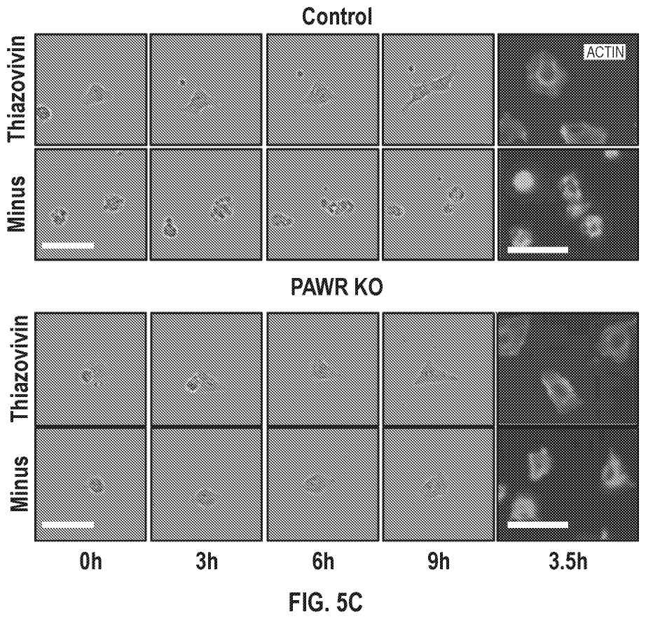

To conduct detailed analysis of PAWR mutants cells were exposed to Cas9 RNPs targeting PAWR and a knockout cell line with a normal karyotype was generated (FIGS. 9A-9F). It was observed that the suppression of dissociation-induced death is specific to PAWR mutants and PMAIP1 mutants were unable to survive passaging without thiazovivin (FIG. 5A-B). Conversely, PAWR mutants were unable to survive DSB-induced toxicity and further demonstrated the specificity of the respective phenotypes (FIG. S9E). Next, it was examined how PAWR mutants survive by taking time-lapse images. After single cell dissociation and treatment with thiazovivin, both control and PAWR mutants survived as single cells by extending cell projections which promoted attachment and survival (FIG. 5C). Control cells without thiazovivin exhibited membrane blebbing and subsequently died (FIG. 5C). Conversely, PAWR mutants without thiazovivin had greatly reduced blebbing and survived as single cells (FIG. 5C). The cytoskeletal organization was further examined using phalloidin to stain filamentous (F—) ACTIN. The thiazovivin treated cells had an increased surface area, a fanned-out shape with actin stress fibers, and a large circular adhesion belt-like structure (FIG. 5C). In the absence of thiazovivin, control cells had many small actin rings which marked membrane blebs. PAWR mutants without thiazovivin exhibited reduced membrane blebbing and small actin rings (FIG. 5C).

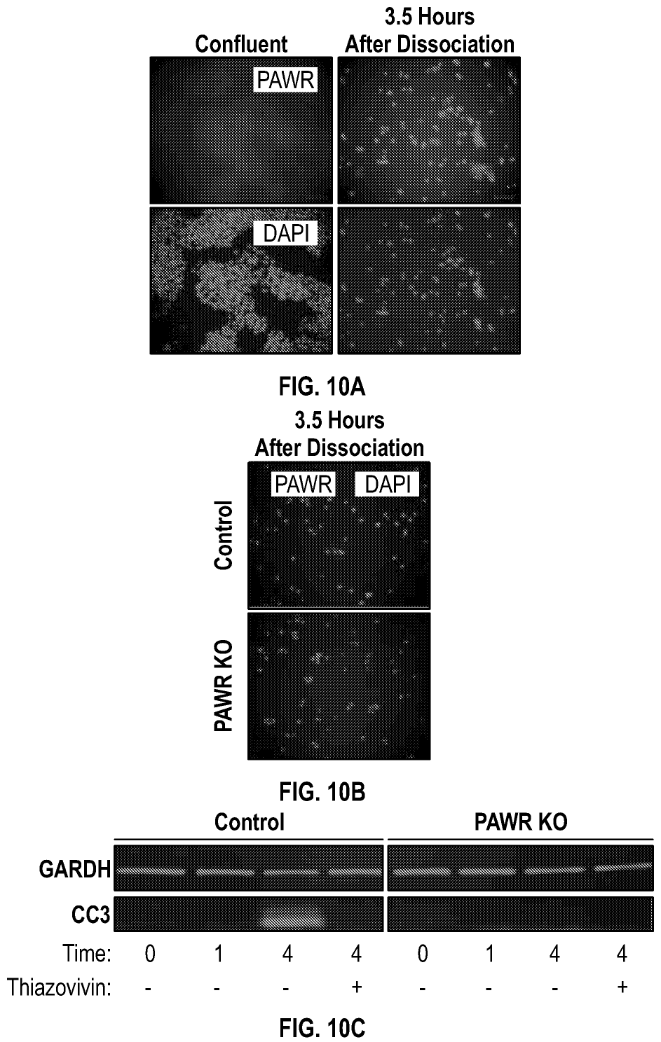

Molecularly, PAWR has dual roles as a transcriptional repressor that causes cell death or as an actin binding protein that regulates contractility (Burikhanov et al., 2009; Johnstone et al., 1996; Vetterkind and Morgan, 2009). The molecular function of PAWR in dissociated hPSCs was investigated using a specific antibody. Despite having abundant PAWR mRNA, PAWR protein is post-trancriptionally regulated and is induced by dissociation in hPSCs (FIGS. 9A-9F and FIGS. 10A-10C). Immunofluorescence did not detect PAWR protein in the nucleus, however, localization of PAWR with F-ACTIN in both thiazovivin treated and untreated cells was observed (FIG. 5D). PAWR localized to adhesion belt-like structures in the presence of thiazovivin and to membrane blebs in the untreated cells after dissociation. Additional hits from the screen, PRKCZ and SCRIB, also localized to membrane blebs. Both PRKCZ and SCRIB are expressed in the early mouse embryo and exhibit a ROCK-dependent cell polarity (Kono et al., 2014). Cumulatively, a novel role for PAWR in dissociation-induced death was identified in these studies. PAWR mutants survived dissociation without ROCK inhibitors because of a failure to initiate membrane blebbing and downstream caspase activation (FIGS. 10A-10C) (Ohgushi et al., 2010). Furthermore, PAWR is a known proapoptotic factor. Data disclosed herein demonstrated that PAWR protein is induced upon dissociation and colocalized with the ACTIN network that is, e.g., responsible for initiating membrane blebbing and subsequent cell death.

### FACS-Based Screen for Regulators of Pluripotency

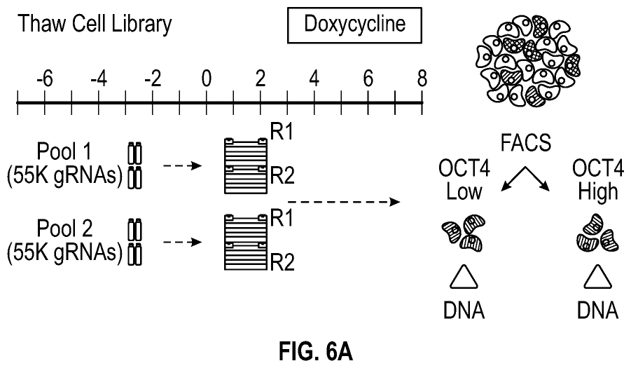

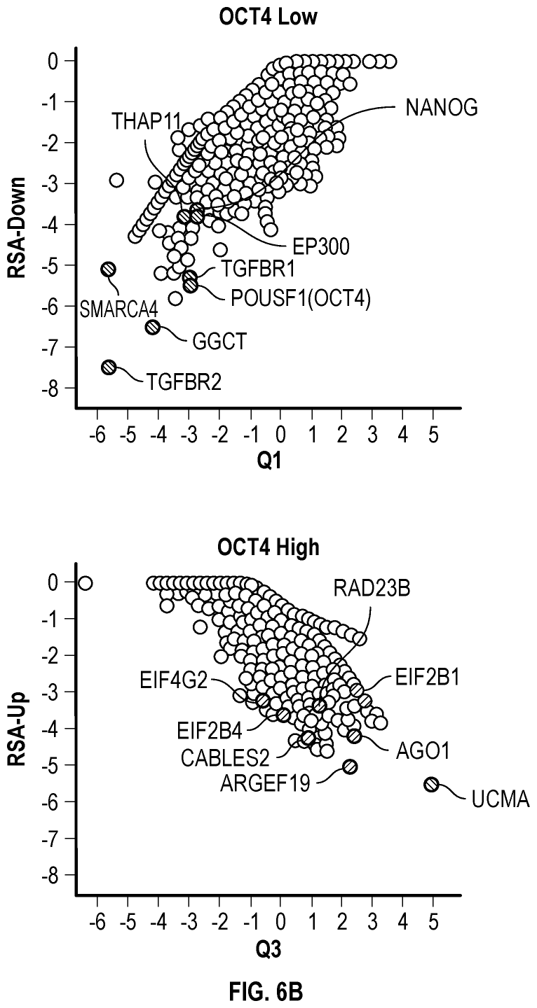

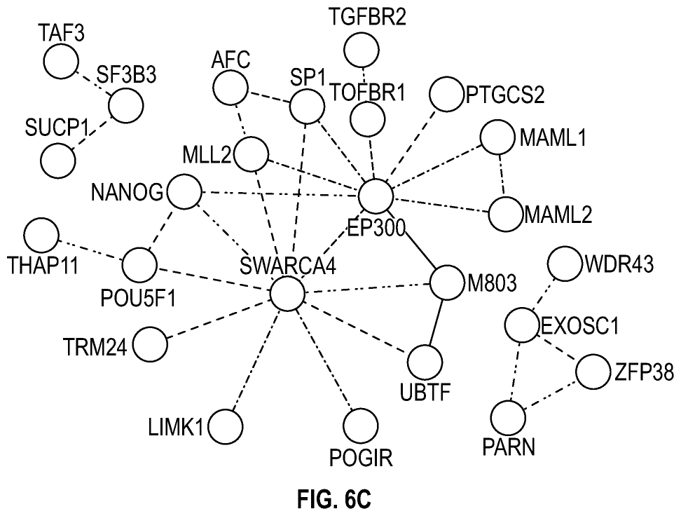

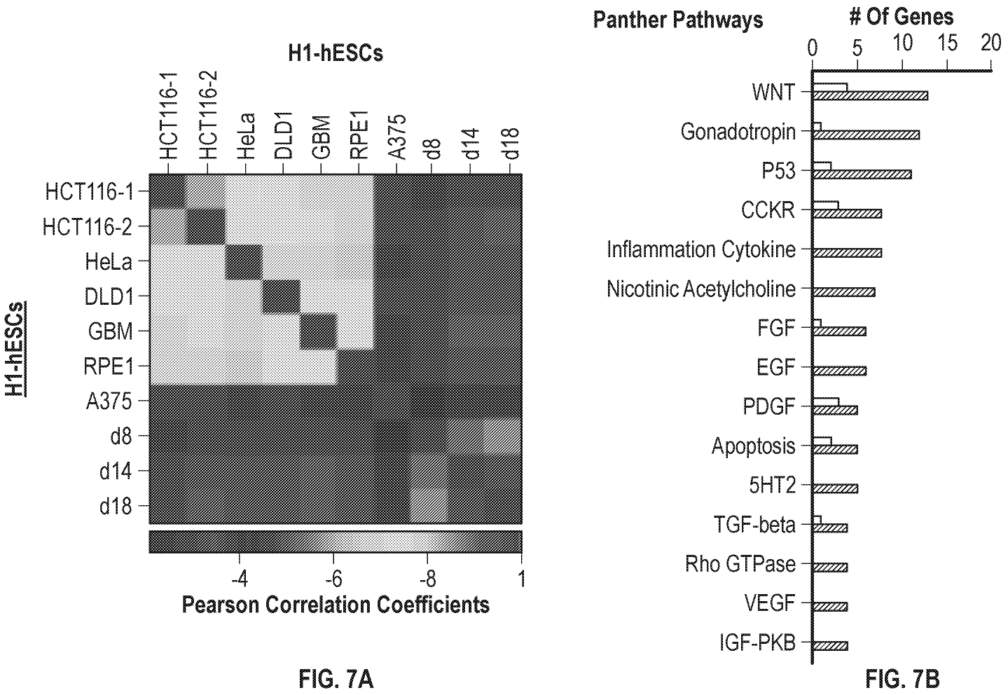

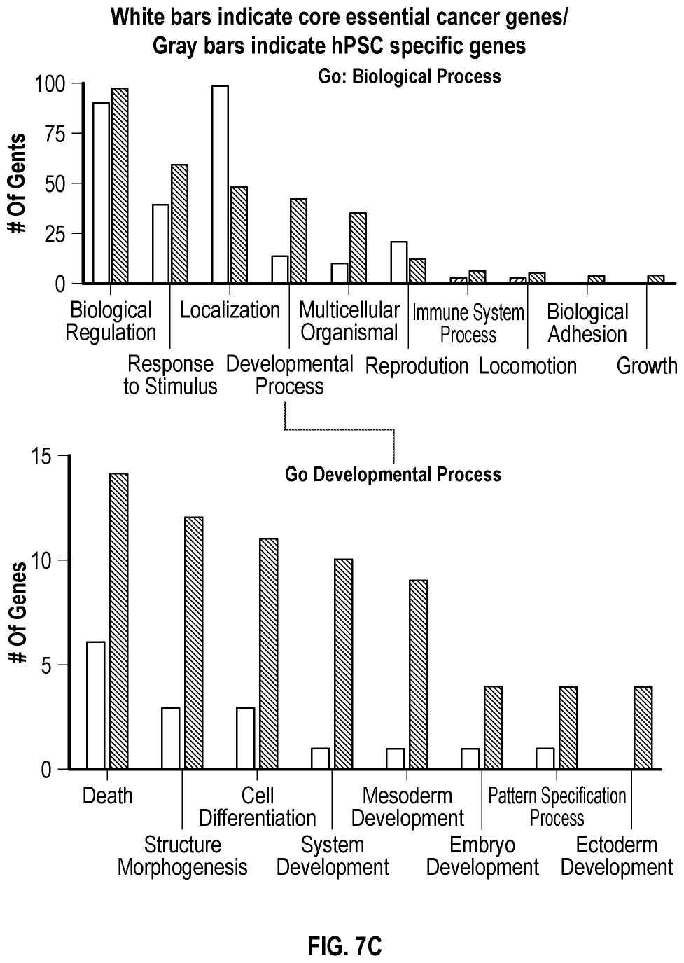

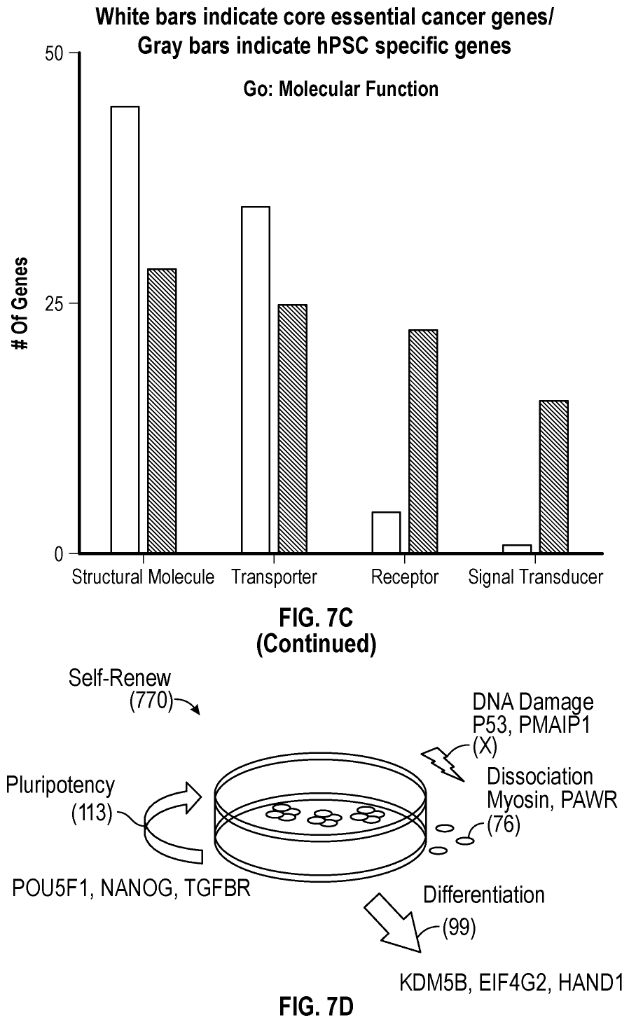

Although our fitness screen detected a significant hPSC-specific dropout of OCT4, other critical regulators of pluripotency like NANOG did not appear to affect fitness (FIG. 1E, and Table 4). A genome scale fitness screen in mESCs had similar results and only reported the dropout of three genes regulating blastocyst development (Koike-Yusa et al., 2014). Pluripotency and cellular fitness of hPSCs may not be related and this indicates, e.g., that some differentiated cell types may not exhibit changes in fitness when cultured in the pluripotent media. To specifically identify regulators of human pluripotency a FACS-based pooled screen using an OCT4 antibody was conducted. One year after conducting the first fitness screen, a genome-scale CRISPR cell library was thawed and expanded prior to conducting the screen. The cells were mutagenized with Cas9 for 8 days prior to FACS sorting, which was used to separate the OCT4^{HIGH} and OCT4^{LOW} expressing populations (FIG. 6A). Log 2(fold change) was calculated by comparing the OCT4^{LOW} group to the OCT4^{HIGH} group. The p-values calculated by the RSA test were plotted against Q1 and Q3 based z-scores for OCT4^{LOW} and OCT4^{HIGH} respectively (FIG. 7B, and Table 8). Importantly, a significant enrichment of OCT4 and NANOG targeting sgRNAs was detected in the OCT4^{LOW} group. An enrichment of TGFBR1/2 genes required to maintain the culture of hPSCs and the chromatin regulators EP300 and SMARCA4/BRG1 which regulate OCT4 expression and function of hPSCs was also detected (Chen et al., 2011; King and Klose, 2017; Singhal et al., 2010; Wang et al., 2012). Using STRING-db a core network of genes connected to OCT4 were identified in the OCT4^{LOW} group highlighting the ability of phenotypic CRISPR screening to identify relevant gene networks (FIG. 6C). Additionally, the OCT4^{HIGH} group identified factors that promote differentiation such as, HAND1, KDM5B, and EIF4G2, (Table 8) (Hough et al., 2006; Kidder et al., 2013; Yamanaka, 2000). In addition to EIF4G2 two translational regulators, EIF2B1 and EIF2B4, were identified in the OCT4^{HIGH} group. Many of the genes in these lists have published roles in regulating pluripotency, reprograming or embryonic development and further investigation of the less studied genes may, e.g., reveal novel insights into the human pluripotent state (Tables 8-9).

Identification of hPSCs Specific Fitness and Pluripotency Gene Networks

Extensive CRISPR screening in cancer cell lines has provided a wealth of knowledge about their genetic dependencies, however, the static state of these cells has revealed less about genes with developmental functions (Hart et al., 2015). To compare hPSC results to cancer cell lines pairwise Pearson correlation coefficients using Bayes factors distributions was conducted (cancer data from Hart et al., 2015). The analysis revealed that hPSCs formed a distinct cluster (FIG. 7A) and is consistent with the partial overlap between core essential genes in cancer and fitness genes in hPSCs (52% FIG. 1E). To focus on gene networks specific to hPSCs bioinformatics analysis was conducted comparing 829 core essentials cancer genes (essential for 5/5 cell lines, Hart et al., 2015) to hPSC-specific gene sets identified by the screen described herein. A total of 653 hPSC-specific genes were obtained from the fitness screen (365), dissociation-induced death screen (76), and the OCT4 FACS screen (212) (Table 10). The gene lists were analyzed using the PANTHER classification system (pantherdb.org). PANTHER pathway analysis identified a greater diversity of 92 enriched pathways in hPSCs and only 38 in the cancer lines (FIG. 7B). In accordance with this an increase in the number of genes with receptor and signal transducer activity molecular functions was also detected (FIG. 7C). The hPSC enriched pathways included several expected regulators; FGF, TGF-Beta, and WNT (FIG. 7B). FGF2 and TGFβ are critical components of E8 media and are required to maintain pluripotency in vitro (Chen et al., 2011). WNT signaling regulates both differentiation and pluripotency in ESCs (Sokol, 2011). Activation of EGF, PDGF, and VEGF was also observed to be important for the maintenance of the pluripotent state in (FIG. 7B) (Brill et al., 2009). hPSCs also exhibited increases in P53 and CCKR/Rho GTPases pathways which regulate apoptosis and have critical roles in determining the sensitivity of hPSCs to DNA damage and enzymatic dissociation (FIG. 7B). Examination of the biological processes gene ontology revealed an increase in the number of development and multicellular organism genes (FIG. 7C). Furthermore, sub-dividing the developmental process category revealed enrichment of genes regulating cell death, differentiation and early developmental stages (FIG. 7C). Globally, these results highlight the identification of cell type-specific genes regulating different aspects of the pluripotent state. By screening for regulators of three fundamental processes governing the culture of hPSCs, the experiments described herein identified known regulators, in addition to novel genes involved in stem cell biology (FIG. 7D).

TABLE 4
hPSC fitness genes

| | |
|---|---|
| 770 fitness | RPS8; POLR3B; CDK12; DAP3; RPL7A; ZNF648; MECR; H3F3A; MCM2; |
| genes | RPL31; RPS3A; DPAGT1; APITD1; CENPW; ZNF322; RPL9; CDIPT; SNRPF; |
| (depleted) | DARS2; HSPD1; SNRPD1; CBWD6; TRIM24; RNF168; ITGAV; XPO5; EXOSC1; |
| | USO1; MRPL28; WDR1; TBRG4; RPS25; CENPM; WDR43; DDX54; RPL36; |
| | CYCS; LRPPRC; IARS; ANAPC10; GABPA; EIF3J; RAD9A; UTP23; COPS5; |
| | PSMB7; POLR1A; FDXR; ICT1; RFT1; RPS16; ATP5A1; CTAGE4; NOP58; POLR2D; |
| | RPL27A; ESPL1; DNTTIP2; DDB1; NAA25; HDAC3; PSMA7; MYC; RPLPO; |
| | RPS13; FEN1; CTAGE9; ZCCHC14; POLR3A; CCDC84; MRPL57; WDR92; |
| | MRPL4; MRPL34; RNGTT; ZBTB11; RRP12; RPL32; NOC4L; LRWD1; RTEL1; |
| | RPAP1; GTF2H1; TPX2; SHQ1; MED30; NCBP1; URB2; DHX30; EXOSC2; RBM8A; |
| | GTPBP10; UBE2G2; TTL; RPP40; PPP2R2A; RPL39L; PSMD2; WAPL; PAFAH1B1; |
| | SERPING1; HSPA5; PPP1R12A; POLR3H; ACTL6A; TIMM10; RFC3; |
| | NOP56; DTL; ELAC2; RPRD1B; RPS20; ZNF131; NDUFB6; EIF1AD; NDUFB4; |
| | HEATR1; UBL5; TUT1; TRMT112; MFN2; GNL2; RPL3; SUPV3L1; MICU3; |
| | MAD2L2; ENO1; WARS; NDUFA8; SCAP; MRPL33; NDUFA4; TOP2A; MAVS; |
| | ZSCAN2; YBEY; CCDC180; HSPE1; CTC1; ALDOA; EEF2; EIF3A; TRAPPC1; |
| | YTHDC1; ZFAT; RPL18; NOL10; HMGCS1; MTRNR2L8; PHF5A; ATRIP; |
| | TBCA; DHFR; WDR76; ATP5D; DDX1; MRPS5; ANKRD20A4; ZNF837; |
| | UBE2S; KL; MRPS16; FECH; NDUFA10; MRPS6; RPL35A; ZGLP1; PGAM1; |
| | SARS; RPLP2; ZNF207; POLE; AGAP2; EIF1AX; NUP85; TARS2; GR5F1; |
| | RUVBL1; DNM1L; SYNC; RPL13; PPP4R2; RPS3; SART1; TANGO6; RPS19; TECR; |
| | SKA3; ELN; NCKAP1; HNRNPC; DDR1; HMGB1; SAR1A; COPS6; CDK9; FTSJ2; |
| | G6PD; CCT4; LUC7L3; RBM14- |
| | RBM4; TIMM23; ALG1; ADRM1; ACLY; BRIP1; PSMA1; COX6B1; RNASEH2A; |
| | SPATA31A3; EIF3CL; RACK1; PITRM1; FADS3; POLR3E; RPL38; CXXC1; |
| | NOB1; FANCD2; NUP50; SF3B5; AKIRIN2; SNRPE; NOM1; EDC4; EIF3C; |
| | C6orf136; RPS6; SNAPC3; GUK1; NDUFA2; PWP1; SLC5A5; PSMC3; SF3B3; |
| | F2; NOP2; PPRC1; MDM2; TM9SF1; OSBPL7; SNU13; PSMD14; PARP1; ARPC4; |
| | AGBL5; POLR1B; STAT5A; KAT8; LIN37; PPP6C; CBWD5; PPP1R16A; |
| | RBM39; DNM2; HCFC1; RPL17; PRPF3; TXNRD1; CBWD3; MRPS11; MED8; |
| | LSM8; SDHD; POP1; STRA13; NSA2; NDC80; PLEKHN1; CAND2; RAD54L; PRUNE; |
| | UCHL5; PAK1IP1; ECD; CDC25A; SAMD4B; ZNF250; SNRNP35; DGCR8; IPO4; |
| | UPF2; FARSA; MIPEP; INTS9; HIST1H2BC; RPL4; MYT1L; FBXO5; EIF4A3; |
| | PTBP1; UBE2Z; MLLT4; MED11; ATP6V1F; SSU72; RPS29; SF3A2; PDCL3; |
| | SPC24; PGK1; PSMD4; UBE2C; CCT5; HSPA14; KAT5; HIST1H2BL; PSMD7; |
| | LRP10; SLC25A51; SLC6Al2; VRTN; DCAF7; NAT9; LRIT2; RPL12; RAPH1; |
| | PSMB4; CREB1; SNAP23; PTMA; HSPA9; GPR31; ARMC12; HJURP; SGF29; |
| | POLR2J; ANKRD35; ZNF622; COQ6; UROD; CDC7; YARS2; SUPT6H; COX17; |
| | NUP214; ERCC1; ARL8B; ATR; TUBA1B; SEPSECS; ILF2; TIMM44; PUM3; |
| | POLQ; RCC1; SNRNP40; TDP1; NUP98; CCNG1; KIAA1211; UBE2I; RBMX; |
| | PTPMT1; SRSF2; CPSF2; DDX23; POU5F1; MCM4; TINTF2; SULT1A4; SULT1A3; |
| | USP5; PPP1CA; POP4; PMVK; PPP4C; ANAPC15; HIST1H2BD; DHPS; |
| | CPSF3L; LAMTOR2; ETF1; EXOSC8; VAC14; MRPL24; HHAT; CHMP2A; PPIAL4D; |
| | METTL1; WAC; NUP43; CHMP3; MARS; ARL2; SLC4A11; PIK3C2B; |
| | PSMG3; HIST1H2BN; HIST2H3C; URM1; NSF; GEMIN5; ELP6; HIST2H3A; |
| | CSMD2; MMS22L; SDE2; BRAT1; PUF60; POLD2; TRAF4; BUD31; PPP2R1A; |
| | BUB1; SERPINE3; MROH7; REV3L; POLR3G; RPS11; CCT8; PTCH2; HYPK; |
| | FANCL; MBTPS1; NARS2; ATG2A; U2AF1; RBM14; MUC1; JMJD6; ElF3H; |
| | MAPRE1; NCL; RPP21; GPR61; QRICH1; CLCN6; UFL1; CDCA8; CLEC11A; |
| | UPF1; TGFBRAP1; DHX16; PARG; ACTR1A; MCM5; PKM; PSTK; SRSF7; NOL6; |
| | PHYKPL; ZSCAN10; ACTL6B; BRD9; NR2C2AP; RPLP1; KANSL3; DCLRE1B; |
| | CDC45; RASA3; ELL; EXOSC7; EIF1; GPS1; GATAD2A; STT3A; MRPS34; |
| | CDC16; ATP1A1; CD27; TUFM; SPTLC2; RNF25; ISY1- |
| | RAB43; TADA2A; ADAM12; ZMIZ1; DKC1; FIGNL1; PDCD2; DHODH; ANAPC5; |
| | POLR2C; DDX56; ACTR8; SLC27A4; TRAPPC5; SPAG11A; COASY; TRIM68; |
| | PTCD3; WNT3; HIST1H2AJ; PRPF38B; KNTC1; ATP6V0C; UNC93A; KIF19; |
| | SPRY2; SMG7; PYROXD1; DNAI1; RPL29; KDM4A; TUBGCP2; ASNA1; |
| | SRBD1; RPN1; MUC3A; THAP4; TRIM74; PSMC4; RPS15; FASN; CEP192; SUDS3; |
| | DGKI; MRPS25; CDC123; TUBD1; KANSL2; DDX18; PCNA; TXNL4A; |
| | PRMT1; NCBP2; KIF18A; H2AFZ; ACTR3; VPS13D; MT2A; SETD1B; SLC7A5; |
| | ZBTB42; TAZ; STIP1; RPL5; WDR46; GNB1L; MRPL47; CEPT1; HIST1H2AI; |
| | UBTF; SNRPD2; COX14; POLR2L; PRODH; GAPDH; WDR89; PSMB6; TXNDC17; |
| | ERCC2; RPL27; MFSD12; SNRPG; PAM16; ESF1; USP8; IRS2; ZNF536; |
| | TPI1; USP1; PPP2CA; EXOSC4; RPL7; RCL1; CHORDC1; BRF2; SP1; RPL19; |
| | HGS; TWISTNB; BRSK2; ENY2; ALG11; SF3A1; RAD51D; ZNF581; TBC1D10C; |
| | SMC2; HSD17B10; FOXD4L6; AURKAIP1; RPS9; MRPL46; SIN3A; NOL12; |
| | CCAR2; SDK1; RPL14; RPL35; WRB; ZNF445; MYLK2; HNRNPK; FGA; TTC37; |
| | MRPS18A; ALYREF; MCMBP; AUNIP; CCDC94; XRN2; SNX17; KNDC1; |
| | CPSF3; CCDC58; AURKB; HARS2; CTIF; URB1; GPN2; ZNF446; ACTB; FAM92B; |
| | CCT7; PPP1CC; GGPS1; KCNJ10; STAC3; RNF103- |
| | CHMP3; TIMELESS; SACM1L; CCNF; PSMA2; LMOD3; SKIV2L2; NAE1; TUBA8; |
| | MYCBP; AAAS; RPL10; CLEC18C; EIF2S1; ANAPC4; KCTD21; THAP12; |
| | KIN; FNTB; ACIN1; RFC4; CCT3; SKIV2L; HNRNPU; RPL11; BTBD2; ARFRP1; |
| | ZC3H11A; TAF3; CHAF1A; TNPO1; SYVN1; PPP1R2; CTR9; COX20; DDX6; |
| | SRSF3; EXOSC5; SPCS2; SRSF1; FASTKD5; COX4I1; RRP7A; FAM136A; |
| | WDR5; ZNF574; CUL5; MCM9; RIC8A; TUBGCP6; PRELID1; LONP1; S100P; |
| | SEMA6B; MCM6; ASUN; STRIP1; MBOAT7; KLHL3; MYH4; EIF6; NUP133; |
| | CHD4; HAUS3; FBXW7; HIST2H3D; EIF2B2; DOHH; CDHR1; SRRT; CMTM5; |
| | HMGCR; USP19; SPTBN4; SAMM50; POLR2I; KPNB1; CD200; OSBPL9; |
| | NDOR1; UNC45A; MRPL20; TSR2; ARIH2; SP8; RDH10; AFG3L2; HLA- |
| | DOB; EIF2B1; AK8; SETD6; PPP2R3C; MAP3K1; PURG; CSDC2; MRPL48; |
| | SYTL1; CD3EAP; HIST2H2BE; COX11; MST1; NSMCE3; TARS; MRPL53; RPS4X; |
| | DNM1; TUBGCP3; RBM19; NUP155; NCAPH; MED18; ZMYND8; RBX1; FOXD4L4; |
| | FRG2C; FAM72B; DNAJC21; CCDC130; HIST1H3B; TMED9; MBD1; |
| | HSPB2; LRCH2; EXOC7; PNN; HAUS5; CBWD1; USP17L21; IL27; DDX47; |
| | ARAP1; AHCTF1; SS18L2; RFC5; TFAP4; SZRD1; RPS14; TXN2; SLC30A9; |
| | CKAP5; EWSR1; MLX; FXN; FAAP100; ADAMTS13; GMEB1; UQCRH; SMAP2; |
| | CEP57; ORC4; BPTF; ZNF347; SALL1; RABGGTB; MRPL21; RPA1; PFDN6; |
| | SNAPC5; MMS19; |
| 405 core | ACLY; ACTL6A; ACTR1A; ACTR3; ACTR8; AFG3L2; AKIRIN2; ALDOA; ALG1; |
| fitness | ALG11; ALYREF; ANAPC10; ANAPC15; ANAPC4; ANAPC5; ARFRP1; |
| genes | ARL2; ARPC4; ASNA1; ATP1A1; ATP5A1; ATP5D; ATP6V0C; ATR; ATRIP; |
| (depleted) | AURKAIP1; AURKB; BRAT1; BRF2; BUD31; CCDC130; CCDC84; CCDC94; |
| | CCT3; CCT4; CCT5; CCT7; CD3EAP; CDC123; CDC16; CDC25A; CDC45; CDCA8; |
| | CDK12; CDK9; CENPM; CENPW; CEP192; CEP57; CHAF1A; CHD4; CHMP2A; |
| | CHMP3; CKAP5; COASY; COPS5; COPS6; COX4I1; COX6B1; CPSF2; CPSF3; |
| | CPSF3L; CTC1; CTR9; DAP3; DARS2; DCAF7; DCLRE1B; DDB1; DDX1; |
| | DDX18; DDX23; DDX47; DDX54; DDX56; DDX6; DGCR8; DHFR; DHODH; |
| | DHPS; DHX16; DKC1; DNM1L; DNM2; DNTTIP2; DPAGT1; DTL; ECD; EEF2; |
| | EIF1; EIF1AD; EIF2B1; EIF2B2; EIF2S1; EIF3A; EIF3H; EIF3J; EIF4A3; |
| | EIF6; ELAC2; ELL; ELP6; ENO1; ERCC1; ERCC2; ESF1; ESPL1; ETF1; EWSR1; |
| | EXOSC1; EXOSC2; EXOSC4; EXOSC5; EXOSC7; EXOSC8; FARSA; FEN1; FNTB; |
| | G6PD; GEMIN5; GGPS1; GNB1L; GNL2; GPN2; GPS1; GUK1; HARS2; HAUS3; |
| | HAUS5; HCFC1; HDAC3; HEATR1; HGS; HJURP; HMGCR; HMGCS1; HNRNPK; |
| | HNRNPU; HSD17B10; HSPA14; HSPA5; HSPA9; IARS; ILF2; INTS9; |
| | ITGAV; KANSL2; KANSL3; KAT8; KIF18A; KIN; KNTC1; KPNB1; LAMTOR2; |
| | LONP1; LRPPRC; LUC7L3; MAD2L2; MARS; MCM2; MCM4; MCM5; MCM6; |
| | MCMBP; MDM2; MECR; MED11; MED18; MED30; METTL1; MFN2; MIPEP; |
| | MMS19; MMS22L; MRPL20; MRPL21; MRPL28; MRPL34; MRPL4; MRPL46; |
| | MRPL47; MRPL53; MRPS11; MRPS18A; MRPS25; MRPS34; MRPS5; MRPS6; |
| | MYC; NAA25; NAE1; NARS2; NCAPH; NCBP1; NCBP2; NCKAP1; NCL; |
| | NDC80; NDOR1; NDUFA2; NDUFB6; NOB1; NOC4L; NOL10; NOL12; NOL6; |
| | NOM1; NOP2; NOP56; NOP58; NR2C2AP; NSA2; NSF; NUP133; NUP155; NUP214; |
| | NUP43; NUP85; NUP98; ORC4; PAFAH1B1; PAK1IP1; PCNA; PFDN6; PGK1; |
| | PKM; PMVK; PNN; POLD2; POLE; POLR1A; POLR1B; POLR2C; POLR2D; |
| | POLR2I; POLR2L; POLR3A; POLR3B; POLR3H; POP1; POP4; PPP1CA; PPP2CA; |
| | PPP2R1A; PPP4C; PPP6C; PPRC1; PRMT1; PRPF3; PRPF38B; PSMA1; |
| | PSMA2; PSMA7; PSMB4; PSMB6; PSMB7; PSMC3; PSMC4; PSMD14; PSMD2; |
| | PSMD4; PSMD7; PSMG3; PSTK; PTCD3; PTPMT1; PUF60; QRICH1; RABGGTB; |
| | RAD51D; RAD9A; RBM14; RBM19; RBMX; RCC1; RCL1; RFC3; RFC4; |
| | RFC5; RFT1; RIC8A; RNASEH2A; RNF168; RNGTT; RPA1; RPAP1; RPL10; |
| | RPL11; RPL12; RPL13; RPL18; RPL19; RPL27A; RPL29; RPL3; RPL31; RPL38; |
| | RPL4; RPL7; RPL9; RPLPO; RPLP2; RPN1; RPP21; RPP40; RPS11; RPS13; |
| | RPS29; RPS4X; RPS9; RRP12; RTEL1; RUVBL1; SACM1L; SAMD4B; SAMM50; |
| | SARS; SART1; SEPSECS; SF3A1; SF3A2; SF3B3; SF3B5; SHQ1; SINT3A; SKA3; |
| | SKIV2L2; SLC30A9; SMC2; SNAPC3; SNRNP35; SNRPD2; SNRPE; SNRPF; SPC24; |
| | SRBD1; SRRT; SRSF1; SRSF2; SRSF7; SS18L2; SSU72; STRIP1; SUDS3; |
| | SUPT6H; SUPV3L1; SYVN1; TANGO6; TARS; TARS2; TBCA; TIMELESS; TIMM10; |
| | TIMM23; TIMM44; TINF2; TNPO1; TOP2A; TPX2; TRAPPC1; TRAPPC5; |
| | TRMT112; TSR2; TUBD1; TUBGCP2; TUBGCP3; TUBGCP6; TUFM; TUT1; |
| | TWISTNB; TXN2; TXNL4A; TXNRD1; U2AF1; UBE2I; UBL5; UBTF; UNC45A; |
| | UPF1; UPF2; URB1; URB2; URM1; UROD; USO1; USP5; USP8; UTP23; VPS13D; |
| | WARS; WDR43; WDR46; WDR5; WDR92; WRB; XPO5; XRN2; YARS2; |
| | YBEY; YTHDC1; ZBTB11; ZNF131; ZNF207; ZNF574; ZNF622; |
| 365 stem | AAAS; ACIN1; ACTB; ACTL6B; ADAM12; ADAMTS13; ADRM1; AGAP2; AGBL5; |
| fitness | AHCTF1; AK8; ANKRD20A4; ANKRD35; APITD1; ARAP1; ARIH2; ARL8B; |
| genes | ARMC12; ASUN; ATG2A; ATP6V1F; AUNIP; BPTF; BRD9; BRIP1; BR5K2; |
| (depleted) | BTBD2; BUB1; C6orf136; CAND2; CBWD1; CBWD3; CBWD5; CBWD6; |
| | CCAR2; CCDC180; CCDC58; CCNF; CCNG1; CCT8; CD200; CD27; CDC7; CDHR1; |
| | CDIPT; CEPT1; CHORDC1; CLCN6; CLEC11A; CLEC18C; CMTM5; COQ6; |
| | COX11; COX14; COX17; COX20; CREB1; CSDC2; CSMD2; CTAGE4; CTAGE9; |
| | CTIF; CUL5; CXXC1; CYCS; DDR1; DGKI; DHX30; DNAI1; DNAJC21; |
| | DNM1; DOHH; EDC4; EIF1AX; EIF3C; EIF3CL; ELN; ENY2; EXOC7; F2; |
| | FAAP100; FADS3; FAM136A; FAM72B; FAM92B; FANCD2; FANCL; FASN; FASTKD5; |
| | FBXO5; FBXW7; FDXR; FECH; FGA; FIGNL1; FOXD4L4; FOXD4L6; |
| | FRG2C; FTSJ2; FXN; GABPA; GAPDH; GATAD2A; GMEB1; GPR31; GPR61; |
| | GRSF1; GTF2H1; GTPBP10; H2AFZ; H3F3A; HHAT; HIST1H2AI; HIST1H2AJ; |
| | HI5T1H2BC; HI5T1H2BD; HIST1H2BL; HIST1H2BN; HI5T1H3B; HIST2H2BE; |
| | HIST2H3A; HIST2H3C; HIST2H3D; HLA- |
| | DOB; HMGB1; HNRNPC; HSPB2; HSPD1; HSPE1; HYPK; ICT1; IL27; IPO4; |
| | IRS2; ISY1- |
| | RAB43; JMJD6; KAT5; KCNJ10; KCTD21; KDM4A; KIAA1211; KIF19; KL; |
| | KLHL3; KNDC1; LIN37; LMOD3; LRCH2; LRIT2; LRP10; LRWD1; LSM8; MAP3K1; |
| | MAPRE1; MAVS; MBD1; MBOAT7; MBTPS1; MCM9; MED8; MFSD12; |
| | MICU3; MLLT4; MLX; MROH7; MRPL24; MRPL33; MRPL48; MRPL57; MRPS16; |
| | MST1; MT2A; MTRNR2L8; MUC1; MUC3A; MYCBP; MYH4; MYLK2; |
| | MYT1L; NAT9; NDUFA10; NDUFA4; NDUFA8; NDUFB4; NSMCE3; NUP50; |
| | OSBPL7; OSBPL9; PAM16; PARG; PARP1; PDCD2; PDCL3; PGAM1; PHF5A; |
| | PHYKPL; PIK3C2B; PITRM1; PLEKHN1; POLQ; POLR2J; POLR3E; POLR3G; |
| | POU5F1; PPIAL4D; PPP1CC; PPP1R12A; PPP1R16A; PPP1R2; PPP2R2A; PPP2R3C; |
| | PPP4R2; PRELID1; PRODH; PRUNE; PTBP1; PTCH2; PTMA; PUM3; PURG; |
| | PWP1; PYROXD1; RACK1; RAD54L; RAPH1; RASA3; RBM14- |
| | RBM4; RBM39; RBM8A; RBX1; RDH10; REV3L; RNF103- |
| | CHMP3; RNF25; RPL14; RPL17; RPL27; RPL32; RPL35; RPL35A; RPL36; RPL39L; |
| | RPL5; RPL7A; RPLP1; RPRD1B; RPS14; RPS15; RPS16; RPS19; RPS20; RPS25; |
| | RPS3; RPS3A; RPS6; RPS8; RRP7A; S100P; SALL1; SAR1A; SCAP; SDE2; |
| | SDHD; SDK1; SEMA6B; SERPINTE3; SERPING1; SETD1B; SETD6; SGF29; |
| | SKIV2L; SLC25A51; SLC27A4; SLC4A11; SLC5A5; SLC6A12; SLC7A5; SMAP2; |
| | SMG7; SNAP23; SNAPC5; SNRNP40; SNRPD1; SNRPG; SNU13; SNX17; SP1; |
| | SP8; SPAG11A; SPATA31A3; SPCS2; SPRY2; SPTBN4; SPTLC2; SRSF3; STAC3; |
| | STAT5A; STIP1; STRA13; STT3A; SULT1A3; SULT1A4; SYNC; SYTL1; |
| | SZRD1; TADA2A; TAF3; TAZ; TBC1D10C; TBRG4; TDP1; TECR; TFAP4; TGFBRAP1; |
| | THAP12; THAP4; TM9SF1; TMED9; TPI1; TRAF4; TRIM24; TRIM68; |
| | TRIM74; TTC37; TTL; TUBA1B; TUBA8; TXNDC17; UBE2C; UBE2G2; UBE2S; |
| | UBE2Z; UCHL5; UFL1; UNC93A; UQCRH; USP1; USP17L21; USP19; VAC14; |
| | VRTN; WAC; WAPL; WDR1; WDR76; WDR89; WNT3; ZBTB42; ZC3H11A; |
| | ZCCHC14; ZFAT; ZGLP1; ZMIZ1; ZMYND8; ZNF250; ZNF322; ZNF347; |
| | ZNF445; ZNF446; ZNF536; ZNF581; ZNF648; ZNF837; ZSCAN10; ZSCAN2; |
| 950 genes | PMAIP1; TP53; OFD1; ZNF729; PLEKHA1; CYP2D6; NXT2; PSG5; PIK3R3; KIFAP3; |
| fitness screen | CCDC6; HYPM; UBE2D1; ZMAT1; CFHR3; GPR50; SLCO1B7; KRT6A; |
| (enriched) | PABPC5; STRC; UBE2NL; PPM1B; GPR34; APPBP2; USP17L10; SARM1; SUFU; |
| | LOC389895; CMC4; SYTL5; TAS2R43; RARA; ZNF275; TEX13C; OR4S2; |
| | PRRG1; ZCCHC16; MAGIX; IL13RA2; GRK6; HSPB11; ZNF180; ZNF846; LILRB3; |
| | NF2; KDM5C; PABPC1L2A; PABPC1L2B; USP6NL; STATH; SPANXN4; |
| | SDCCAG8; UGT2B17; HHLA2; HMGN5; RAB9A; BRS3; F9; SPCS1; GSTT1; |
| | ELAVL1; FOXI3; ZC4H2; PRAMEF1; ADH1B; FAM127B; ZNF555; ARL13A; |
| | ZNF443; MYL10; S100G; SATL1; ACSM2B; MAGEB18; BHLHB9; DEFB121; |
| | MAP7D2; RPGR; PRKX; MICU2; NRK; ACTL10; IFNL2; ZNF845; FIGF; FOXQ1; |
| | CSNK2A3; KCNE5; SH3BGRL; CTXN3; ZNF679; S100A7A; KIAA2026; |
| | SMIM9; SFTPA2; SPOP; IFRD1; LANCL3; GPR174; CDKL5; HLA- |
| | DRB1; APLN; TSPAN8; MAOA; JADE3; AVPR1A; ZNF716; ZNF302; HLA- |
| | DRB5; CHEK2; ZNF676; CDX4; CDY1; CDY1B; STK40; ERI2; KRTAP9- |
| | 6; PTGFR; IFT80; PGRMC1; RBM15; FAM9A; ENOX2; LGALS8; FAM9C; MPC1L; |
| | HPR; ANKRD30B; LOC649238; PUDP; GSK3B; EDA2R; RBP7; DIRC1; PBOV1; |
| | CD40LG; NECTIN3; IFT88; C5orf63; SERPINB3; AHR; SLC25A14; DUSP9; |
| | OR1C1; KDELC2; ZNF721; PCNX4; RNF128; PLGRKT; CASK; RS1; ACTRT1; |
| | CYP2A6; NRG4; PRKCQ; AKAP4; FUT3; MAMLD1; EGFL6; DHRS4; LONRF3; |
| | SYCP2; RLIM; LCE3C; MAP3K15; GAGE10; C8orf48; NOM03; TXNDC8; |
| | OR13H1; TIGD2; PSG2; EFHC2; MIR1307; SIGLEC11; TTC21B; LTA; CCL4; |
| | KLHL15; TNFRSF19; NROB1; SRD5A1; POTEC; VSIG4; HSBP1L1; NXF5; LDOC1; |
| | ADGRF2; N4BP2L2; SERPINTB11; ZCCHC12; ZNF611; PTGS1; ZNF839; |
| | AMY2A; DPYD; RPL36A; TMEM257; LCA10; ITGAD; CXorf56; ZNF713; APOBEC3F; |
| | BBIP1; CD63; ZNF75D; LCE4A; FAM111A; AMELY; MBNL3; MSL3; |
| | SPEN; FKBP5; VPS11; FAM127A; DCAF12L1; TMEM164; ZBED5; SALL2; |
| | CYBB; ZNF799; PTPN12; STXBP4; SLFN12L; ZNF280C; ZNF645; TMEM99; |
| | PLP1; DCAF8; EFCAB9; CEP83; DGKK; STAT2; KLRK1; TBCEL; FRA10AC1; |
| | SULT1A2; TCEAL7; CXCL10; CXorf58; PCDHA8; INPPL1; ZNF711; SAT1; |
| | PRORY; BCOR; GBP1; TRIM74; DOCK11; XKR3; MS4A13; MS4A13; TUBA3E; |
| | KLF2; EIF1AY; TLR3; ZNF559; SLC39A3; PADI4; CFAP43; LDHA; HIST3H3; |
| | OR6A2; DMD; ZNF740; ARMCX5; ARMCX1; SUGT1; SIX6; PRH2; MEIG1; |
| | ARHGAP28; MXRA5; HLA- |
| | DQA1; TSACC; XK; ZNF182; IL1RAPL1; MIR604; FAM47A; FAAH2; CCDC79; |
| | REPS2; SLC22A8; MPHOSPH6; C9orf84; RNASE2; BMX; DYNLT3; NPIPB3; |
| | CD46; ARHGEF17; CNGA2; LPAR3; KRTAP9- |
| | 3; BEND2; SLC6A14; HNRNPCL2; SEMG1; TAAR8; TTC22; CSHL1; CPXCR1; |
| | HECW2; GPC4; THEM6; YIPF6; GLYATL1; TPST2; ZNF208; AP3S2; PDLIM5; |
| | RAB40A; DCAF8L1; EPHA7; FAHD2A; KRT39; PJA1; PLCXD3; IFT74; RGS5; |
| | CCDC152; RAI2; QARS; ATG4A; KIR2DL1; EVX2; DBX2; MID1; RGAG4; |
| | RHCE; RAB33A; EN2; PSG11; PRR21; GBX2; CSDE1; ST6GAL2; SLITRK4; |
| | ZNF548; SNTB1; SERPINB7; ZNF559- |
| | ZNF177; KLRC3; BCORL1; YPEL2; DNAJC19; GPR82; ELM01; DYNC2111; |
| | FOXR2; CRYGD; ZIC3; TRIP12; PRM3; CNTNAP3B; POTEE; MIR3621; SLC12A2; |
| | ZC3H12B; KHDC1; SAA1; YOD1; DEFB128; TCEAL9; NPY5R; SFTPA1; |
| | CYSLTR1; P2RX6; STEAP2; COX7B2; MAGEA1; LPAR4; HRASLS; MAGED1; |
| | BMP15; P2RY10; PIEZO2; AMMECR1; ZNF665; GRPR; UBALD2; DCTD; RPS4Y2; |
| | BST2; TAS1R2; PXMP2; TVP23B; CCDC160; MAGEB10; DNASE2B; |
| | SH2D1A; F8A1; F8A3; F8A2; METTL11B; TGIF2LY; HCAR3; TSPOAP1; |
| | MAP7D3; PDZD4; EYA4; FAM47C; CDH13; COL10A1; LCE3B; GYPB; PATE1; |
| | KLHL23; IDH3B; OPN1LW; ARHGAP26; TCEANC; ZNF649; PLCL1; KLRF1; |
| | PFKFB1; DAB1; RAB41; TOPORS; ACTR10; ANKRD20A1; IFNA7; RAD50; |
| | GRIA3; CYLC1; SPANXN5; CELA3A; GZMK; CMTR2; BEX3; IAPP; PRKAA2; |
| | SEC24A; SLC16A2; SPATS2L; SP140L; CXCL11; SCRT1; FBX042; SPANXN3; |
| | CCL15; TPD52L2; PACRGL; TMEM47; NAP1L3; TMEM231; HSFX1; RAB9B; |
| | AQP4; RAD21L1; L3MBTL3; OVCH2; SP140; SHTN1; SH3KBP1; ROPN1B; |
| | BIRC8; ZMYM2; LRRFIP2; OR4C15; F8; RASA1; MAGEA3; PSG8; OR51L1; |
| | FRMD7; NCK1; COX6C; GPC3; HLA- |
| | B; CEACAM3; TMEM86A; HTR2C; MAGEH1; STEAP1; ZNF628; PRELID3A; |
| | ASICS; FLG2; ANKRD26; SPA17; CES4A; ZNF81; ADAM21; RBMXL2; HCAR2; |
| | HNF4G; NELL2; UBE2L6; BRWD1; ZNF16; KRTAP10- |
| | 4; UBL4A; ZAK; MSX1; NLN; ZC3H12D; NUDT6; CLEC9A; TNFSF10; MUC21; |
| | CUL3; NAP1L2; GLA; KIF3A; CABP5; UBE2E1; SLC15A5; DUSP16; AP1AR; |
| | SSX2B; SSX2; WDR34;; LHB; NHSL2; GDI1; LILRA2; CEACAM21; PIBF1; |
| | NHLH1; HP; SLITRK2; DCAF12L2; GSKIP; MTX1; STRAP; CCL23; PRAMEF2; |
| | CNGA1; ZNF345; TAS2R50; RP2; HADHB; LGALS7B; LGALS7; CHM; |
| | TMEM185A; CANX; KRTAP20- |
| | 1; MIR1294; SPACA5; SPACA5B; CST3; CCNB3; PLS3; IGSF1; CHST9; APOOL; |
| | CDKN2D; TMBIM4; CEP19; DTX3L; CYP11B2; ZNF256; STK24; SPANXB1; |
| | TMEM35; PDK1; USP3; SGTB; TMEM31; ARMCX4; F5; PASD1; CAPN8; |
| | ZNF175; TBC1D15; ZNF638; TC2N; ADGRG4; HLA- |
| | E; GABRE; ARL5A; HNMT; IFT81; OR4K14; ZNF442; TMEM109; CFL2; VGLL1; |
| | KMT5B; PDE4D; TRIM71; PDCL2; HDX; PHF6; CNTLN; CTPS2; AGAP3; |
| | HHEX; SMIM10; GADD45G; FTHL17; SP5; SYT16; PAGE5; CLEC2B; POLM; |
| | DDX60; OTC; LRRC6; ZXDA; STEAP1B; HNRNPH2; CTLA4; FAXC; SLC1A7; |
| | LSM5; NATD1; ITSN2; OR52N4; CNKSR2; FHL1; ELF4; USP27X; PCDHGA9; |
| | GUCA1C; TANC1; FOPNL; NKX2- |
| | 6; LAYN; NBAS; SPATS1; SPEM1; AMN1; LIN28A; CR2; IFIT3; MAGT1; PDK3; |
| | ARL4C; DYX1C1; SERPINA7; HIST3H2A; OR4K5; CCDC188; CDK2AP1; ZNF491; |
| | OR52R1; OPHN1; S100A7; SPINT3; RIMBP3; CTAG1B; CTAG1A; HOMER2; |
| | TMEM27; DTX2; ZNF146; PQLC3; TCF7L2; METTL9; RNASE12; PPFIA1; |
| | FLAD1; HAUS6; MYL1; PPP1R42; BPY2; BPY2B; BPY2C; DAZ1; DAZ4; |
| | DAZ2; DHRS4L2; RGCC; SMCO2; ADGRL2; MOS; ORM1; SND1; MIR615; RSG1; |
| | PRDM5; NCR1; CASP6; ICOS; TMEM161B; ZDHHC15; TCEAL2; RNF43; |
| | GYG2; POU3F4; ACSL4; C7orf62; DUSP6; PCDH11Y; TMSB4Y; CDK16; CXCL5; |
| | SOX1; PSMB5; TKTL1; MIR4645; NMRK1; ARSH; ERAS; GPRASP1; MKKS; |
| | FAM200B; SSR4; TNFSF9; PATE4; PTCHD1; LYSMD3; CCDC121; RGS17; |
| | IL6ST; RHEBL1; FLT3LG; PRDM8; LRRD1; PLEKHB2; AFF2; ENO4; GABRA3; |
| | SPRED1; SLCO1A2; ZAN; ZAN; FAM163B; IGFL3; DGKH; MAP6; MUM1L1; |
| | SAGE1; SHROOM4; RHOBTB3; CCDC87; CRYGB; GK; PPP3CB; NYX; |
| | ZNF483; CPNE3; GPR22; SLC10A3; KRTAP21-2; GSTA2; FXYD6- |
| | FXYD2; MECP2; KIR2DL3; SYAP1; COL27A1; CAPN6; WDR72; SLC16A5; |
| | STK38L; MTM1; ZDHHC9; FSD1L; S100Al2; IL18R1; SPDYE2; FGG; LDLR; |
| | MOB2; ATXN3L; DLX1; SMARCA1; STK26; OR5D14; FPR2; CD274; SBNO2; |
| | LRRC39; CORO2B; ARMC10; CEP76; AGAP7P; AGAP7P; MNDA; MAMDC4; |
| | ADGRB1; KIAA1107; LILRA6; GALR1; KRTAP1-1; ZNF470; JOSD2; HLA- |
| | DQA2; CHPT1; PIGK; DPP9- |
| | AS1; CYP4Z1; TLR1; ATP5J; ATP5J; TMEM56; CFHR2; EIF2AK1; DEDD; FGFR4; |
| | EXT1; MIR934; MORC4; MCF2L2; MYOT; NLGN3; SPTAN1; WAS; DND1; |
| | FAM160A2; SLC32A1; CXorf40A; CXorf40B; SLC5A4; ACSM2A; SDHB; |
| | C5orf28; C5AR2; SLC30A4; ZNF285; LPAR6; GJB7; NDP; BEX5; EPOR; |
| | GATA6; TAF7L; TRIM50; ZNF728; PHF14; TRDMT1; COX8A; FAM46D; KRTAP19- |
| | 2; DSC3; FOXD3; TBL1Y; EPHA10; TBX22; THUMPD3; CNRIP1; MIR432; |
| | CYP2A13;; OR4C11; SLC27A2; KRTAP10- |
| | 5; SRRM3; ARL14EPL; FBXO45; ZNF449; RYBP; ZNF669; CHCHD4; MOSPD1; |
| | ZFY; PDCD1; LAGE3; SOC55; NXF3; ZNF573; CCDC122; FAT4; POMZP3; |
| | APNK2; SEMA3A; KIAA1211L; KCNN2; PHOSPHO2- |
| | KLHL23; PRKACB; DDAH1; PBDC1; CLDN2; DLG3; SLC25A53; CNNM3; |
| | AOC3; PIH1D3; ARMCX5- |
| | GPRASP2; LCE1F; CRTC3; SLC25A23; RUNX2; FABP2; TH; PTEN; PSD3; |
| | ZNF630; ANKRD31; ZADH2; ARR3; LOC100288336; TAB2; MAGEC1; BEX2; |
| | UBE2G1; KCNAB3; UNC13C; NMUR1; FZD8; SNX12; RAVER1; KRT81; LOC730183; |
| | ARMCX6; CFHR1; KIAA1644; HOXB7; BCL2L11; CHRDL1; SPPL2B; |
| | HPGD; CHAT; PCDHA13; BNC2; RORC; MAGEC2; LELP1; CT45A5; CYP3A43; |
| | SSX7; BBS2; HMBOX1; PCSK1N; GUCY1B3; HOXC8; RBM41; HTR1F; RSF1; |
| | CBR1; MOSPD2; TCTE3; BCAP31; CYP39A1; NLGN4X; RFPL2; CHRNA3; |
| | SPAG1; ZCCHC18; ACRV1; ADAM2; ZNF420; GPR160; HEPHL1; ZCRB1; |
| | HOXC9; FGF13; ARL6; KCTD4; TMLHE; OR10J1; FBXW11; BIK; NFIB; FCGR2B; |
| | CACNG6; ADGRA2; |

TABLE 6
TP53 related genes

| | p53 | |
|---|---|---|
| symbol | enriched | chromosome |
| CDKN2D | yes | 19 |
| CHEK2 | yes | 22 |
| DTX3L | yes | 3 |
| EDA2R | yes | X |
| ELAVL1 | yes | 19 |
| GADD45G | yes | 9 |
| GSK3B | yes | 3 |
| LGALS7B | yes | 19 |
| PDK1 | yes | 2 |
| PIK3R3 | yes | 1 |
| PMAIP1 | yes | 18 |
| POLM | yes | 7 |
| PSMB5 | yes | 14 |
| PTEN | yes | 10 |
| RAD50 | yes | 5 |
| TOPORS | yes | 9 |
| TP53 | yes | 17 |
| UBE2D1 | yes | 10 |
| BIK | yes | 22 |
| BCL2L11 | yes | 2 |

TABLE 7
DID activator, loss of which results in suppression of DID

| SYMBOL | CATEGORY |
|---|---|
| ACO1 | DID activator |
| MPHOSPH6 | DID activator |
| SUPT20H | DID activator |
| ABCC11 | DID activator |
| ALPP | DID activator |
| AMY2A | DID activator |
| ARHGEF5 | DID activator |
| ARL3 | DID activator |
| ATP7A | DID activator |
| ATRN | DID activator |
| C10orf76 | DID activator |
| C9orf3 | DID activator |
| CD300C | DID activator |
| CDK16 | DID activator |
| CDKN2D | DID activator |
| CDY1 | DID activator |
| CDY1B | DID activator |
| CFHR2 | DID activator |
| CHEK2 | DID activator |
| CHRNA6 | DID activator |
| DAPK3 | DID activator |
| EFCAB5 | DID activator |
| EIF4B | DID activator |
| EPOR | DID activator |
| FAM13B | DID activator |
| FLII | DID activator |
| GSTK1 | DID activator |
| HIST1H4H | DID activator |
| IPP | DID activator |
| KATNAL1 | DID activator |
| KIF3A | DID activator |
| KIR2DL3 | DID activator |
| LIN28A | DID activator |
| LRG1 | DID activator |
| LYPD8 | DID activator |
| MAT2A | DID activator |
| MDM4 | DID activator |
| MYH9 | DID activator |
| MYL6 | DID activator |
| MYL9 | DID activator |
| NFYB | DID activator |
| OPHN1 | DID activator |
| PAWR | DID activator |
| PIGB | DID activator |
| PLCH1 | DID activator |
| PLEKHA1 | DID activator |
| PMAIP1 | DID activator |
| PPP2R1B | DID activator |
| PTEN | DID activator |
| RLIM | DID activator |
| ROCK1 | DID activator |
| SCRIB | DID activator |
| SERPINA1 | DID activator |
| SERPINB10 | DID activator |
| SH3BGRL | DID activator |
| SHD | DID activator |
| SHOC2 | DID activator |
| SIAH1 | DID activator |
| SIGLEC11 | DID activator |
| SLCO1B7 | DID activator |
| ST8SIA3 | DID activator |
| STRAP | DID activator |
| SYTL5 | DID activator |
| TADA2B | DID activator |
| TEX11 | DID activator |
| TP53 | DID activator |
| VTCN1 | DID activator |
| ZNF154 | DID activator |
| ZNF25 | DID activator |
| ZNF285 | DID activator |
| ZNF436 | DID activator |
| ZNF654 | DID activator |
| HNMT | DID activator |
| IDNK | DID activator |
| IFRD1 | DID activator |
| SLCO1A2 | DID activator |

TABLE 8
OCT4 HIGH and OCT4 LOW group

| SYMBOL | CATEGORY |
|---|---|
| EIF2B1 | OCT HIGH |
| RPLP0 | OCT HIGH |
| POP4 | OCT HIGH |
| MYCBP | OCT HIGH |
| PHKG1 | OCT HIGH |
| ACTG2 | OCT HIGH |
| KYAT1 | OCT HIGH |
| CYP1B1 | OCT HIGH |
| EIF4G2 | OCT HIGH |
| GNRH2 | OCT HIGH |
| GTF3C1 | OCT HIGH |
| HSF1 | OCT HIGH |
| IL9R | OCT HIGH |
| INHBB | OCT HIGH |
| LSS | OCT HIGH |
| MC3R | OCT HIGH |
| ORC1 | OCT HIGH |
| POLH | OCT HIGH |
| PRKAB2 | OCT HIGH |
| RAD23B | OCT HIGH |
| SCN9A | OCT HIGH |
| SLCO2A1 | OCT HIGH |
| CLDN14 | OCT HIGH |
| HIST1H3I | OCT HIGH |
| SPOP | OCT HIGH |
| ASMTL | OCT HIGH |
| HYAL2 | OCT HIGH |
| MPDZ | OCT HIGH |
| EIF2B4 | OCT HIGH |
| HAND1 | OCT HIGH |
| ADAMTS1 | OCT HIGH |
| SLC25A44 | OCT HIGH |
| FRAT1 | OCT HIGH |
| MAEA | OCT HIGH |
| BASP1 | OCT HIGH |
| GNLY | OCT HIGH |
| KDM5B | OCT HIGH |
| CLPX | OCT HIGH |
| GPR75 | OCT HIGH |
| AP4S1 | OCT HIGH |
| PTGDR2 | OCT HIGH |
| ZFPM2 | OCT HIGH |
| SS18L1 | OCT HIGH |
| CCDC9 | OCT HIGH |
| FAM162A | OCT HIGH |
| AGO1 | OCT HIGH |
| FETUB | OCT HIGH |
| TOX3 | OCT HIGH |
| HOOK2 | OCT HIGH |
| BICRA | OCT HIGH |
| RDH8 | OCT HIGH |
| TACO1 | OCT HIGH |
| PHF20 | OCT HIGH |
| CDKL3 | OCT HIGH |
| FKBP11 | OCT HIGH |
| TNFRSF12A | OCT HIGH |
| LINS1 | OCT HIGH |
| SFT2D3 | OCT HIGH |
| FAM104A | OCT HIGH |
| DIRC2 | OCT HIGH |
| DDX49 | OCT HIGH |
| SLC35E3 | OCT HIGH |
| SLC48A1 | OCT HIGH |
| ERGIC1 | OCT HIGH |
| DLGAP3 | OCT HIGH |
| ARHGEF28 | OCT HIGH |
| SLC7A10 | OCT HIGH |
| TMUB2 | OCT HIGH |
| GGNBP2 | OCT HIGH |
| TMEM62 | OCT HIGH |
| MAP3K19 | OCT HIGH |
| CABLES2 | OCT HIGH |
| TMUB1 | OCT HIGH |
| FAM96A | OCT HIGH |
| LIN52 | OCT HIGH |
| GNG8 | OCT HIGH |
| ODF3 | OCT HIGH |
| CYGB | OCT HIGH |
| SLC22A12 | OCT HIGH |
| B4GALNT2 | OCT HIGH |
| LOXHD1 | OCT HIGH |
| ARHGEF19 | OCT HIGH |
| TRIM43 | OCT HIGH |
| ZNF721 | OCT HIGH |
| ZSWIM2 | OCT HIGH |
| IGSF23 | OCT HIGH |
| C1orf127 | OCT HIGH |
| UCMA | OCT HIGH |
| C6orf89 | OCT HIGH |
| MYADML2 | OCT HIGH |
| MAGI3 | OCT HIGH |
| KCNRG | OCT HIGH |
| TREML4 | OCT HIGH |
| LCTL | OCT HIGH |
| KRTAP5-3 | OCT HIGH |
| LIN28B | OCT HIGH |
| ALKAL1 | OCT HIGH |
| OR13J1 | OCT HIGH |
| LCN10 | OCT HIGH |
| UBTF | OCT4 LOW |
| SF3B3 | OCT4 LOW |
| WDR43 | OCT4 LOW |
| EXOSC1 | OCT4 LOW |
| XPO5 | OCT4 LOW |
| POU5F1 | OCT4 LOW |
| SP1 | OCT4 LOW |
| TRIM24 | OCT4 LOW |
| CSDC2 | OCT4 LOW |
| TAF3 | OCT4 LOW |
| ARMC12 | OCT4 LOW |
| PARN | OCT4 LOW |
| PDGFRL | OCT4 LOW |
| PDE6B | OCT4 LOW |
| APC | OCT4 LOW |
| CHRM3 | OCT4 LOW |
| CYP2D6 | OCT4 LOW |
| DDOST | OCT4 LOW |
| DRP2 | OCT4 LOW |
| EP300 | OCT4 LOW |
| GRP | OCT4 LOW |
| TLX1 | OCT4 LOW |
| LIMK1 | OCT4 LOW |
| MAGEB3 | OCT4 LOW |
| MOCS1 | OCT4 LOW |
| MUC6 | OCT4 LOW |
| NFRKB | OCT4 LOW |
| NRAP | OCT4 LOW |
| PTGIR | OCT4 LOW |
| PTPRM | OCT4 LOW |
| SLC4A2 | OCT4 LOW |
| SMARCA4 | OCT4 LOW |
| TBX2 | OCT4 LOW |
| TGFBR1 | OCT4 LOW |
| TGFBR2 | OCT4 LOW |
| ZFP36 | OCT4 LOW |
| KMT2D | OCT4 LOW |
| FZD7 | OCT4 LOW |
| SLC5A6 | OCT4 LOW |
| USP17L13 | OCT4 LOW |
| USP17L17 | OCT4 LOW |
| STOML1 | OCT4 LOW |
| SOX13 | OCT4 LOW |
| BCLAF1 | OCT4 LOW |
| MAML1 | OCT4 LOW |
| GIT2 | OCT4 LOW |
| CNIH1 | OCT4 LOW |
| ARPP19 | OCT4 LOW |
| FTCD | OCT4 LOW |
| RUNDC3A | OCT4 LOW |
| ZZEF1 | OCT4 LOW |
| TCTN3 | OCT4 LOW |
| TTLL3 | OCT4 LOW |
| AK5 | OCT4 LOW |
| CHIC2 | OCT4 LOW |
| TOR2A | OCT4 LOW |
| THYN1 | OCT4 LOW |
| PKN3 | OCT4 LOW |
| PADI1 | OCT4 LOW |
| ZNF580 | OCT4 LOW |
| RHCG | OCT4 LOW |
| MBD3 | OCT4 LOW |
| FBXO34 | OCT4 LOW |
| SUSD4 | OCT4 LOW |
| MAML2 | OCT4 LOW |
| ZNF469 | OCT4 LOW |
| MFSD2A | OCT4 LOW |
| PLXDC2 | OCT4 LOW |
| HMGCLL1 | OCT4 LOW |
| SLC39A4 | OCT4 LOW |
| THAP11 | OCT4 LOW |
| SUGP1 | OCT4 LOW |
| PPCDC | OCT4 LOW |
| PROK2 | OCT4 LOW |
| SEMA4A | OCT4 LOW |
| INPP5E | OCT4 LOW |
| RSRP1 | OCT4 LOW |
| GGCT | OCT4 LOW |
| VPS37B | OCT4 LOW |
| NANOG | OCT4 LOW |
| PTGES2 | OCT4 LOW |
| EDEM3 | OCT4 LOW |
| TMEM177 | OCT4 LOW |
| TSPAN14 | OCT4 LOW |
| ROPN1L | OCT4 LOW |
| MINDY4 | OCT4 LOW |
| TMEM107 | OCT4 LOW |
| PAQR8 | OCT4 LOW |
| ARHGAP18 | OCT4 LOW |
| BTBD9 | OCT4 LOW |
| RASGRP4 | OCT4 LOW |
| TSR3 | OCT4 LOW |
| PHACTR3 | OCT4 LOW |
| SLC36A4 | OCT4 LOW |
| TNFAIP8L1 | OCT4 LOW |
| PAQR7 | OCT4 LOW |
| DNAJB8 | OCT4 LOW |
| GPR156 | OCT4 LOW |
| ASPRV1 | OCT4 LOW |
| DNAH2 | OCT4 LOW |
| CDRT15 | OCT4 LOW |
| EME1 | OCT4 LOW |
| FAM47E-STBD1 | OCT4 LOW |
| GDPD4 | OCT4 LOW |
| LRRC73 | OCT4 LOW |
| JAZF1 | OCT4 LOW |
| FUOM | OCT4 LOW |
| TRIM65 | OCT4 LOW |
| CCDC144NL | OCT4 LOW |
| TFAP2E | OCT4 LOW |
| SPATC1 | OCT4 LOW |
| OC90 | OCT4 LOW |
| TMEM242 | OCT4 LOW |

TABLE 9

| | |
|---|---|
| gene | reference |

OCT4 LOW

| | |
|---|---|
| EP300 | Wang W P et al., PLoS One. 2012; 7(12): e52556 |
| FZD7 | Fernandez A. et al., PNAS (2014); 111 (4) 1409-1414 |
| INPP5E | Dyson J M et al., J Cell Biol (2016), jcb.201511055 |
| KMT2D | Wang C et al., PNAS Oct. 18, 2016. 113 (42) 11871-11876 |
| LIMK1 | Xiao Y et al., JBC (2016) doi: 10.1074/jbc.M116.759886; |
| | Duan X et al., Histochem Cell Biol. 2014 August; 142(2): 227-33 |
| MBD3 | Zhang L et al., J. Cell. Mol. Med., 20: 1150-1158 |
| NANOG | Chambers I et al., Nature (2004) 450, pages 1230-1234 |
| NFRKB | Chia N Y et al., Nature (2010) 468, pages 316-320 |
| POU5F1 | Nichols, Jennifer et al., Cell, Volume 95, Issue 3, 379-391 |
| SF3B3 | Jincho Y et al., Biology of Reproduction, Volume 78, Issue 4, |
| | 1 Apr. 2008, Pages 568-576 |
| SMARCA4 | King H W and Klose R J, eLife 2017; 6: e22631 |
| SP1 | Wu D Y and Yao Z, Cell Research volume 16, pages 319-322 (2006) |
| TAF3 | Liu Z et al., Cell, Volume 146, Issue 5, 2011, Pages 720-731, |
| TGFBR1 | Chen G et al., Nature Methods volume 8, pages 424-429 (2011) |
| TGFBR2 | Chen G et al., Nature Methods volume 8, pages 424-429 (2011) |
| THAP11 | Durruthy-Durruthy J et al., Dev Cell. 2016 Jul. 11; 38(1): 100-15. |
| TRIM24 | Rafiee M R et al., Mol Cell. 2016 Nov. 3; 64(3): 624-635 |
| UBTF | Woolnough J L et al., PLOS ONE 11(6): e0157276 |
| ZFP36 | Tan F E and Elowitz M B, PNAS 2014, 111 (17) E1740-E1748 |

OCT4 HIGH

| | |
|---|---|
| ADAMTS1 | Kaeser M D et al., J. Biol. Chem. doi: 10.1074/jbc.M806061200 |
| BASP1 | Blanchard J W et al., Nature Biotechnology volume 35, pages 960-968 (2017) |
| EIF4G2 | Yamanaka S et al., EMBO Journal (2000) 19, 5533-5541 |
| FETUB | Bansho Y et al., (2017) FEBS Lett, 591: 1584-1600. |
| GGNBP2 | Li S et al., Biology of Reproduction, Volume 94, Issue 2, 1 Feb. 2016, 41, 1-12, |
| HAND1 | Hough S R et al., (2006) STEM CELLS, 24: 1467-1475 |
| HSF1 | Byun K et al., Stem Cell Research [8 Sep. 2013, 11(3): 1323-1334] |
| KDM5B | Kidder B L et al., Mol. Cell. Biol. December 2013 vol. 33 no. 24 4793-4810 |
| ZFPM2 | Fu J D et al., Stem Cell Reports, Volume 1, Issue 3, 235-247 |

TABLE 10
Stem cell genes

| Category | Symbol |
|---|---|
| Dissociation | ABCC11; ACO1; ALPP; AMY2A; ARHGEF5; ARL3; ATP7A; ATRN; |
| induced | C10orf76; C9orf3; CD300C; CDK16; CDKN2D; CDY1; CDY1B; CFHR2; |
| death | CHEK2; CHRNA6; DAPK3; EFCAB5; EIF4B; EPOR; FAM13B; FLII; |
| | GSTK1; HIST1H4H; HNMT; IDNK; IFRD1; IPP; KATNAL1; KIF3A; |
| | KIR2DL3; LIN28A; LRG1; LYPD8; MAT2A; MDM4; MPHOSPH6; MYH9; |
| | MYL6; MYL9; NFYB; OPHN1; PAWR; PIGB; PLCH1; PLEKHA1; |
| | PMAIP1; PPP2R1B; PTEN; RLIM; ROCK1; SCRIB; SERPINA1; |
| | SERPINB10; SH3BGRL; SHD; SHOC2; SIAH1; SIGLEC11; SLCO1A2; |
| | SLCO1B7; ST8SIA3; STRAP; SUPT20H; SYTL5; TADA2B; TEX11; |
| | TP53; VTCN1; ZNF154; ZNF25; ZNF285; ZNF436; ZNF654; |
| OCT4HIGH | ACTG2; ADAMTS1; AGO1; ALKAL1; AP4S1; ARHGEF19; ARHGEF28; |
| | ASMTL; B4GALNT2; BASP1; BICRA; C1orf127; C6orf89; CABLES2; |
| | CCDC9; CDKL3; CLDN14; CLPX; CYGB; CYP1B1; DDX49; DIRC2; |
| | DLGAP3; EIF2B1; EIF2B4; EIF4G2; ERGIC1; FAM104A; FAM162A; |
| | FAM96A; FETUB; FKBP11; FRAT1; GGNBP2; GNG8; GNLY; GNRH2; |
| | GPR75; GTF3C1; HAND1; HIST1H3I; HOOK2; HSF1; HYAL2; |
| | IGSF23; IL9R; INHBB; KCNRG; KDM5B; KRTAP5-3; KYAT1; |
| | LCN10; LCTL; LIN28B; LIN52; LINS1; LOXHD1; LSS; MAEA; |
| | MAGI3; MAP3K19; MC3R; MPDZ; MYADML2; MYCBP; ODF3; OR13J1; |
| | ORC1; PHF20; PHKG1; POLH; POP4; PRKAB2; PTGDR2; RAD23B; |
| | RDH8; RPLP0; SCN9A; SFT2D3; SLC22A12; SLC25A44; SLC35E3; |
| | SLC48A1; SLC7A10; SLCO2A1; SPOP; SS18L1; TACO1; TMEM62; |
| | TMUB1; TMUB2; TNFRSF12A; TOX3; TREML4; TRIM43; UCMA; |
| | ZFPM2; ZNF721; ZSWIM2;; |
| OCT4LOW | AK5; APC; ARHGAP18; ARMC12; ARPP19; ASPRV1; BCLAF1; BTBD9; |
| | CCDC144NL; CDRT15; CHIC2; CHRM3; CNIH1; CSDC2; CYP2D6; |
| | DDOST; DNAH2; DNAJB8; DRP2; EDEM3; EME1; EP300; EXOSC1; |
| | FAM47E-STBD1; FBXO34; FTCD; FUOM; FZD7; GDPD4; GGCT; GIT2; |
| | GPR156; GRP; HMGCLL1; INPP5E; JAZF1; KMT2D; LIMK1; LRRC73; |
| | MAGEB3; MAML1; MAML2; MBD3; MFSD2A; MINDY4; MOCS1; MUC6; |
| | NANOG; NFRKB; NRAP; OC90; PADI1; PAQR7; PAQR8; PARN; PDE6B; |
| | PDGFRL; PHACTR3; PKN3; PLXDC2; POU5F1; PPCDC; PROK2; PTGES2; |
| | PTGIR; PTPRM; RASGRP4; RHCG; ROPN1L; RSRP1; RUNDC3A; |
| | SEMA4A; SF3B3; SLC36A4; SLC39A4; SLC4A2; SLC5A6; SMARCA4; |
| | SOX13; SP1; SPATC1; STOML1; SUGP1; SUSD4; TAF3; TBX2; |
| | TCTN3; TFAP2E; TGFBR1; TGFBR2; THAP11; THYN1; TLX1; |
| | TMEM107; TMEM177; TMEM242; TNFAIP8L1; TOR2A; TRIM24; |
| | TRIM65; TSPAN14; TSR3; TTLL3; UBTF; USP17L13; USP17L17; |
| | VPS37B; WDR43; XPO5; ZFP36; ZNF469; ZNF580; ZZEF1; |
| P53 | BCL2L11; BIK; CDKN2D; CHEK2; DTX3L; EDA2R; ELAVL1; |
| related | GADD45G; GSK3B; LGALS7B; PDK1; PIK3R3; PMAIP1; POLM; |
| | PSMB5; PTEN; RAD50; TOPORS; TP53; UBE2D1; |
| Stem cell | AAAS; ACIN1; ACTB; ACTL6B; ADAM12; ADAMTS13; ADRM1; |
| essential | AGAP2; AGBL5; AHCTF1; AK8; ANKRD20A4; ANKRD35; APITD1; |
| | ARAP1; ARIH2; ARL8B; ARMC12; ASUN; ATG2A; ATP6V1F; AUNIP; |
| | BPTF; BRD9; BRIP1; BRSK2; BTBD2; BUB1; C6orf136; CAND2; |
| | CBWD1; CBWD3; CBWD5; CBWD6; CCAR2; CCDC180; CCDC58; CCNF; |
| | CCNG1; CCT8; CD200; CD27; CDC7; CDHR1; CDIPT; CEPT1; |
| | CHORDC1; CLCN6; CLEC11A; CLEC18C; CMTM5; COQ6; COX11; |
| | COX14; COX17; COX20; CREB1; CSDC2; CSMD2; CTAGE4; CTAGE9; |
| | CTIF; CUL5; CXXC1; CYCS; DDR1; DGKI; DHX30; DNAI1; DNAJC21; |
| | DNM1; DOHH; EDC4; EIF1AX; EIF3C; EIF3CL; ELN; ENY2; EXOC7; |
| | F2; FAAP100; FADS3; FAM136A; FAM72B; FAM92B; FANCD2; |
| | FANCL; FASN; FASTKD5; FBXO5; FBXW7; FDXR; FECH; FGA; |
| | FIGNL1; FOXD4L4; FOXD4L6; FRG2C; FTSJ2; FXN; GABPA; GAPDH; |
| | GATAD2A; GMEB1; GPR31; GPR61; GRSF1; GTF2H1; GTPBP10; |
| | H2AFZ; H3F3A; HHAT; HIST1H2AI; HIST1H2AJ; HIST1H2BC; |
| | HIST1H2BD; HIST1H2BL; HIST1H2BN; HIST1H3B; HIST2H2BE; |
| | HIST2H3A; HIST2H3C; HIST2H3D; HLA-DOB; HMGB1; HNRNPC; |
| | HSPB2; HSPD1; HSPE1; HYPK; ICT1; IL27; IPO4; IRS2; ISY1-RAB43; |
| | JMJD6; KAT5; KCNJ10; KCTD21; KDM4A; KIAA1211; KIF19; |
| | KL; KLHL3; KNDC1; LIN37; LMOD3; LRCH2; LRIT2; LRP10; LRWD1; |
| | LSM8; MAP3K1; MAPRE1; MAVS; MBD1; MBOAT7; MBTPS1; |
| | MCM9; MED8; MFSD12; MICU3; MLLT4; MLX; MROH7; MRPL24; |
| | MRPL33; MRPL48; MRPL57; MRPS16; MST1; MT2A; MTRNR2L8; |
| | MUC1; MUC3A; MYCBP; MYH4; MYLK2; MYT1L; NAT9; NDUFA10; |
| | NDUFA4; NDUFA8; NDUFB4; NSMCE3; NUP50; OSBPL7; OSBPL9; |
| | PAM16; PARG; PARP1; PDCD2; PDCL3; PGAM1; PHF5A; PHYKPL; |
| | PIK3C2B; PITRM1; PLEKHN1; POLQ; POLR2J; POLR3E; POLR3G; |
| | POU5F1; PPIAL4D; PPP1CC; PPP1R12A; PPP1R16A; PPP1R2; |
| | PPP2R2A; PPP2R3C; PPP4R2; PRELID1; PRODH; PRUNE; PTBP1; |
| | PTCH2; PTMA; PUM3; PURG; PWP1; PYROXD1; RACK1; RAD54L; |
| | RAPH1; RASA3; RBM14-RBM4; RBM39; RBM8A; RBX1; RDH10; |
| | REV3L; RNF103-CHMP3; RNF25; RPL14; RPL17; RPL27; RPL32; |
| | RPL35; RPL35A; RPL36; RPL39L; RPL5; RPL7A; RPLP1; RPRD1B; |
| | RPS14; RPS15; RPS16; RPS19; RPS20; RPS25; RPS3; RPS3A; |
| | RPS6; RPS8; RRP7A; S100P; SALL1; SAR1A; SCAP; SDE2; SDHD; |
| | SDK1; SEMA6B; SERPINE3; SERPING1; SETD1B; SETD6; SGF29; |
| | SKIV2L; SLC25A51; SLC27A4; SLC4A11; SLC5A5; SLC6A12; |
| | SLC7A5; SMAP2; SMG7; SNAP23; SNAPC5; SNRNP40; SNRPD1; |
| | SNRPG; SNU13; SNX17; SP1; SP8; SPAG11A; SPATA31A3; SPCS2; |
| | SPRY2; SPTBN4; SPTLC2; SRSF3; STAC3; STAT5A; STIP1; |
| | STRA13; STT3A; SULT1A3; SULT1A4; SYNC; SYTL1; SZRD1; |
| | TADA2A; TAF3; TAZ; TBC1D10C; TBRG4; TDP1; TECR; TFAP4; |
| | TGFBRAP1; THAP12; THAP4; TM9SF1; TMED9; TPI1; TRAF4; |
| | TRIM24; TRIM68; TRIM74; TTC37; TTL; TUBA1B; TUBA8; TXNDC17; |
| | UBE2C; UBE2G2; UBE2S; UBE2Z; UCHL5; UFL1; UNC93A; UQCRH; |
| | USP1; USP17L21; USP19; VAC14; VRTN; WAC; WAPL; WDR1; |
| | WDR76; WDR89; WNT3; ZBTB42; ZC3H11A; ZCCHC14; ZFAT; ZGLP1; |
| | ZMIZ1; ZMYND8; ZNF250; ZNF322; ZNF347; ZNF445; ZNF446; |
| | ZNF536; ZNF581; ZNF648; ZNF837; ZSCAN10; ZSCAN2; |

### Discussion

The use of hPSCs in large-scale functional genomics studies has been limited by technical constraints. Prior to the study described herein, it was unclear that genome-scale CRISPR screens were possible in hPSCs as the first attempts had poor performance (Hart et al., 2014; Shalem et al., 2014). This was overcome by building a high performance 2-component CRISPR/CAS9 system for hPSCs. The performance, across many sgRNAs, made the platform amenable to high-throughput screening. Using iCas9 in hPSCs it was possible to perturb hundreds of genes in arrayed format or the entire genome in pooled format. The system is renewable and a pool of stem cells with sgRNAs to the entire genome can be banked, distributed and utilized for successive screens in hPSCs and their differentiated progeny. Beyond technical proficiency, genes that regulate fundamental stem cell processes such as self-renewal, their inherent sensitivity to DNA damage, single cell cloning, pluripotency and differentiation were identified.

Firstly, 770 genes required for the self-renewal of hPSC were identified. A majority of these genes have established roles in fitness while 365 of these genes are novel and specific to hPSCs. This set of genes could be used, e.g., to inform a systematic approach to improve the consistency, robustness and user-friendliness of hPSC culture conditions. During the fitness screen, it was also determined that Cas9 activity imposes a selective pressure on DNA damage sensitive hPSCs. This caused an enrichment of 20 genes that are connected to TP53. Consistent with this dominant negative TP53 mutations and deletions recurrently occur and provide a selective advantage during the culture of hPSCs (Amir and Laurent, 2016; Merkle et al., 2017). In addition to TP53, a hPSC-specific role for PMAIP1 was identified in determining the extreme sensitivity of hPSCs to DNA damage. Like TP53, deletions of chromosome 18 spanning the PMAIP1 locus have been recurrently observed during hPSC culture (Amps et al., 2011) and suggest that PMAIP1 deletion may be responsible for enhanced survival of these lines. These TP53 related genes have the potential to improve the efficiency or safety of genome engineering through transient inhibition or by monitoring their spontaneous mutation rate during hPSC culture (Ihry et al., 2017; Merkle et al., 2017).

Secondly, 76 genes that enhanced the survival of hPSCs during single cell dissociation were identified. Collectively, the screen uncovered an ACTIN/MYOSIN network required for membrane blebbing and cell death caused by dissociation. A novel role for PAWR, a pro-apoptotic regulator that is induced upon dissociation was identified. PAWR is required for membrane blebbing and subsequent death of dissociated hPSCs in the absence of ROCK inhibitors. Importantly, the results disclosed herein are, e.g., developmentally relevant, and the studies further identified SCRIB and PRKCZ which are known regulators of cell polarity in the early mouse embryo (Kono et al., 2014). PAWR has been shown to physically interact with PRKCZ and suggests a potential link between cell polarity and dissociation-induced death (Diaz-Meco et al., 1996). These hits appear to be related to the polarized epiblast-like state of primed hPSC and could explain, e.g., why polarized hPSCs are sensitive to dissociation whereas unpolarized naïve mESC are not (Takashima et al., 2015). Lastly, this set of genes could enable focused approaches to improve the single cell cloning efficiencies of hPSCs and the culture of naïve hPSCs.

The final screen identified 113 genes that are required to maintain pluripotency and 99 genes that potentially regulate differentiation by subjecting the hPSC CRISPR library to FACS sorting on OCT4 protein. Overall, the screen identified an entire network of genes related to OCT4. Nineteen of these genes have previously indicated roles in pluripotency, embryo development and reprogramming (Table 10). Future studies on the novel genes in the list can, e.g., yield new insights about the genetic control of pluripotency and differentiation. These gene sets could, e.g., guide rational improvements to protocols for the maintenance, differentiation and reprogramming of hPSCs.

Overall, the results highlight the ability of unbiased genome-scale screens to identify critical and novel regulators of human pluripotent stem cell biology. Future investigation into the gene sets provided here may be a step toward the genetic dissection of the human pluripotent state. The results described herein, provide scalable work flows that can lower the entry barrier for additional labs to conduct large-scale CRISPR screens in hPSCs. The scalable and bankable platform described here is a renewable resource that can allow for successive screens and the distribution of CRISPR infected hPSC libraries. The platform could potentially be used to improve the generation, culture and differentiation capacity of hPSC. It can also, e.g., generally be applied to the study of development and disease in a wide variety of differentiated cell-types. Established protocols for neurons, astrocytes, cardiomyocytes, hepatocytes and beta-cells can be, e.g., exploited to dissect the genetic nature of development and homeostasis in disease relevant cell-types. This resource may open the door, e.g., for the systematic genetic dissection of disease relevant human cells.

### BIBLIOGRAPHY

Bibliography for Example 1.
Aguirre, A. J., Meyers, R. M., Weir, B. A., Vazquez, F., Zhang, C.-Z., Ben-David, U., Cook, A., Ha, G., Harrington, W. F., Doshi, M. B., et al. (2016). Genomic copy number dictates a gene-independent cell response to CRISPR-Cas9 targeting. Cancer Discov. 2641, 617-632.
Amir, H., and Laurent, L. C. (2016). Spontaneous Single-Copy Loss of TP53 in Human Embryonic Stem Cells Markedly Increases Cell Proliferation and Survival HADAR. 2098-2110.
Amps, K., Andrews, P. W., Anyfantis, G., Armstrong, L., Avery, S., Baharvand, H., Baker, J., Baker, D., Munoz, M. B., Beil, S., et al. (2011). Screening ethnically diverse human embryonic stem cells identifies a chromosome 20 minimal amplicon conferring growth advantage. Nat. Biotechnol. 29, 1132-1144.
Brill, L. M., Xiong, W., Lee, K.-B., Ficarro, S. B., Crain, A., Xu, Y., Terskikh, A., Snyder, E. Y., and Ding, S. (2009). Phosphoproteomic Analysis of Human Embryonic Stem Cells. Cell Stem Cell 5, 204-213.
Burikhanov, R., Zhao, Y., Goswami, A., Qiu, S., Schwarze, S. R., and Rangnekar, V. M. (2009). The Tumor Suppressor Par-4 Activates an Extrinsic Pathway for Apoptosis. Cell 138, 377-388.
Chambers, I., Silva, J., Colby, D., Nichols, J., Nijmeijer, B., Robertson, M., Vrana, J., Jones, K., Grotewold, L., and Smith, A. (2007). Nanog safeguards pluripotency and mediates germline development. Nature 450, 1230-1234.
Chen, G., Hou, Z., Gulbranson, D. R., and Thomson, J. A. (2010). Actin-myosin contractility is responsible for the reduced viability of dissociated human embryonic stem cells. Cell Stem Cell 7, 240-248.
Chen, G., Gulbranson, D. R., Hou, Z., Bolin, J. M., Ruotti, V., Probasco, M. D., Smuga-Otto, K., Howden, S. E., Diol, N. R., Propson, N. E., et al. (2011). Chemically defined conditions for human iPSC derivation and culture. Nat. Methods 8, 424-429.
Chia, N., Chan, Y., Feng, B., Lu, X., Orlov, Y. L., Moreau, D., Kumar, P., Yang, L., Jiang, J., Lau, M., et al. (2010). A genome-wide RNAi screen reveals determinants of human embryonic stem cell identity. Nature 468, 316-320.
Cong, L., Ran, F. A., Cox, D., Lin, S., Barretto, R., Habib, N., Hsu, P. D., Wu, X., Jiang, W., Marraffini, L. A., et al. (2013). Multiplex genome engineering using CRISPR/Cas systems. Science 339, 819-823.
DasGupta, R., Kaykas, A., Moon, R., and Perrimon, N. (2005). Functional genomic analysis of the Wnt-wingless signaling pathway. Science (80-.). 308, 826-833.
Diaz-Meco, M. T., Municio, M. M., Frutos, S., Sanchez, P., Lozano, J., Sanz, L., and Moscat, J. (1996). The product of par-4, a gene induced during apoptosis, interacts selectively with the atypical isoforms of protein kinase C. Cell 86, 777-786.
Echeverri, C. J., Beachy, P. A., Baum, B., Boutros, M., Buchholz, F., Chanda, S. K., Downward, J., Ellenberg, J., Fraser, A. G., Hacohen, N., et al. (2006). Minimizing the risk of reporting false positives in large-scale RNAi screens. Nat. Methods 3, 777-779.
Gutekunst, M., Mueller, T., Weilbacher, A., Dengler, M. A., Bedke, J., Kruck, S., Oren, M., Aulitzky, W. E., and Van Der Kuip, H. (2013). Cisplatin hypersensitivity of testicular germ cell tumors is determined by high constitutive noxa levels mediated by oct-4. Cancer Res. 73, 1460-1469.
Haapaniemi, E., Botla, S., Persson, J., Schmierer, B., and Taipale, J. (2017). Inhibition of p53 improves CRISPR/Cas-mediated precision genome editing Main text.
Hart, T., and Moffat, J. (2016). BAGEL: A computational framework for identifying essential genes from pooled library screens. BMC Bioinformatics.
Hart, T., Brown, K. R., Sircoulomb, F., Rottapel, R., and Moffat, J. (2014). Measuring error rates in genomic perturbation screens: gold standards for human functional genomics. Mol Syst Biol 10.
Hart, T., Chandrashekhar, M., Aregger, M., Durocher, D., Angers, S., Moffat, J., Crispr, H., Hart, T., Chandrashekhar, M., Aregger, M., et al. (2015). High-Resolution CRISPR Screens Reveal Fitness Genes and Genotype-Specific Cancer Liabilities Screens Reveal Fitness Genes. Cell 1-12.
Hebbar, N., Wang, C., and Rangnekar, V. M. (2012). Mechanisms of apoptosis by the tumor suppressor Par-4. J. Cell. Physiol. 227, 3715-3721.
Hough, S. R., Clements, I., Welch, P. J., and Wiederholt, K. A. (2006). Differentiation of Mouse Embryonic Stem Cells after RNA Interference-Mediated Silencing of OCT4 and Nanog. Stem Cells 24, 1467-1475.
Ihry, R. J., Worringer, K. A., Salick, M. R., Frias, E., Ho, D., Kommineni, S., Chen, J., Sondey, M., Ye, C., Randhawa, R., et al. (2017). P53 toxicity is a hurdle to CRISPR/CAS9 screening and engineering in human pluripotent stem cells.
Jinek, M., Chylinski, K., Fonfara, I., Hauer, M., Doudna, J. A., and Charpentier, E. (2012). A Programmable Dual-RNA—Guided DNA Endonuclease in Adaptice Bacterial Immunity. Science 337, 816-822.
Johnstone, R. W., See, R. H., Sells, S. F., Wang, J., Muthukkumar, S., Englert, C., Haber, D. A., Licht, J. D., Sugrue, S. P., Roberts, T., et al. (1996). A novel repressor, par-4, modulates transcription and growth suppression functions of the Wilms' tumor suppressor WT1. Mol. Cell. Biol. 16, 6945-6956.
Kampmann, M., Horlbeck, M. A., Chen, Y., Tsai, J. C., Bassik, M. C., Gilbert, L. A., Villalta, J. E., Kwon, S. C., Chang, H., Kim, V. N., et al. (2015). Next-generation libraries for robust RNA interference-based genome-wide screens. Proc. Natl. Acad. Sci. 112, E3384-E3391.
Khandanpour, C., Phelan, J. D., Vassen, L., Schiitte, J., Chen, R., Horman, S. R., Gaudreau, M. C., Krongold, J., Zhu, J., Paul, W. E., et al. (2013). Growth Factor Independence 1 Antagonizes a p53-Induced DNA Damage Response Pathway in Lymphoblastic Leukemia. Cancer Cell 23, 200-214.
Kidder, B. L., Hu, G., Yu, Z.-X., Liu, C., and Zhao, K. (2013). Extended Self-Renewal and Accelerated Reprogramming in the Absence of Kdm5b. Mol. Cell. Biol. 33, 4793-4810.
Kim, H., Rafiuddin-Shah, M., Tu, H. C., Jeffers, J. R., Zambetti, G. P., Hsieh, J. J. D., and Cheng, E. H. Y. (2006). Hierarchical regulation of mitochondrion-dependent apoptosis by BCL-2 subfamilies. Nat. Cell Biol. 8, 1348-1358.
King, H. W., and Klose, R. J. (2017). The pioneer factor OCT4 requires the chromatin remodeller BRG1 to support gene regulatory element function in mouse embryonic stem cells. Elife 6, 1-24.
Koike-Yusa, H., Li, Y., Tan, E. P., Velasco-Herrera, M. D. C., and Yusa, K. (2014). Genome-wide recessive genetic screening in mammalian cells with a lentiviral CRISPR-guide RNA library. Nat. Biotechnol. 32, 267-273.
Konig, R., Chiang, C., Tu, B. P., Yan, S. F., DeJesus, P. D., Romero, A., Bergauer, T., Orth, A., Krueger, U., Zhou, Y., et al. (2007). A probability-based approach for the analysis of large-scale RNAi screens. Nat. Methods 4, 847-849.
Kono, K., Tamashiro, D. A. A., and Alarcon, V. B. (2014). Inhibition of RHO-ROCK signaling enhances ICM and suppresses TE characteristics through activation of Hippo signaling in the mouse blastocyst. Dev. Biol.
Laurent, L. C., Ulitsky, I., Slavin, I., Tran, H., Schork, A., Morey, R., Lynch, C., Harness, J. V., Lee, S., Barrero, M. J., et al. (2011). Dynamic changes in the copy number of pluripotency and cell proliferation genes in human ESCs and iPSCs during reprogramming and time in culture. Cell Stem Cell 8, 106-118.
Mali, P., Yang, L., Esvelt, K. M., Aach, J., Guell, M., DiCarlo, J. E., Norville, J. E., Church, G. M., Wiedenheft, B., Sternberg, S. H., et al. (2013). RNA-guided human genome engineering via Cas9. Science 339, 823-826.
Mallon, B. S., Chenoweth, J. G., Johnson, K. R., Hamilton, R. S., Tesar, P. J., Yavatkar, A. S., Tyson, L. J., Park, K., Chen, K. G., Fann, Y. C., et al. (2013). StemCellDB: The Human Pluripotent Stem Cell Database at the National Institutes of Health. Stem Cell Res. 10, 57-66.
Masui, S., Nakatake, Y., Toyooka, Y., Shimosato, D., Yagi, R., Takahashi, K., Okochi, H., Okuda, A., Matoba, R., Sharov, A. A., et al. (2007). Pluripotency governed by Sox2 via regulation of Oct3/4 expression in mouse embryonic stem cells. Nat. Cell Biol. 9, 625-635.
McDonald, E. R., de Weck, A., Schlabach, M. R., Billy, E., Mavrakis, K. J., Hoffman, G. R., Belur, D., Castelletti, D., Frias, E., Gampa, K., et al. (2017). Project DRIVE: A Compendium of Cancer Dependencies and Synthetic Lethal Relationships Uncovered by Large-Scale, Deep RNAi Screening. Cell 170, 577-592.e10.
Merkle, F. T., Neuhausser, W. M., Santos, D., Valen, E., Gagnon, J. A., Maas, K., Sandoe, J., Schier, A. F., and Eggan, K. (2015). Efficient CRISPR-Cas9-Mediated Generation of Knockin Human Pluripotent Stem Cells Lacking Undesired Mutations at the Targeted Locus. Cell Rep. 11, 875-883.
Merkle, F. T., Ghosh, S., Kamitaki, N., Mitchell, J., Avior, Y., Mello, C., Kashin, S., Mekhoubad, S., Ilic, D., Charlton, M., et al. (2017). Human pluripotent stem cells recurrently acquire and expand dominant negative P53 mutations. Nature 1-11.
Meyers, R. M., Bryan, J. G., McFarland, J. M., Weir, B. A., Sizemore, A. E., Xu, H., Dharia, N. V., Montgomery, P. G., Cowley, G. S., Pantel, S., et al. (2017). Computational correction of copy number effect improves specificity of CRISPR-Cas9 essentiality screens in cancer cells. Nat. Genet. 49, 1779-1784.
Munoz, D. M., Cassiani, P. J., Li, L., Billy, E., Korn, J. M., Jones, M. D., Golji, J., Ruddy, D. A., Yu, K., McAllister, G., et al. (2016). CRISPR screens provide a comprehensive assessment of cancer vulnerabilities but generate false-positive hits for highly amplified genomic regions. Cancer Discov.
Nichols, J., Zevnik, B., Anastassiadis, K., Niwa, H., Klewe-Nebenius, D., Chambers, I., Scholer, H., and Smith, 5A. (1998). Formation of pluripotent stem cells in the mammalian embryo dependes on the POU transcription factor Oct4. Cell 95,379-391.
Ohgushi, M., Matsumura, M., Eiraku, M., Murakami, K., Aramaki, T., Nishiyama, A., Muguruma, K., Nakano, T., Suga, H., Ueno, M., et al. (2010). Molecular pathway and cell state responsible for dissociation-induced apoptosis in human pluripotent stem cells. Cell Stem Cell 7, 225-239.
Perciavalle, R. M., Stewart, D. P., Koss, B., Lynch, J., Milasta, S., Bathina, M., Temirov, J., Cleland, M. M., Pelletier, S., Schuetz, J. D., et al. (2012). Anti-apoptotic MCL-1 localizes to the mitochondrial matrix and couples mitochondrial fusionÂ to respiration. Nat. Cell Biol. 14,575-583.
Ploner, C., Kofler, R., and Villunger, a (2008). Noxa: at the tip of the balance between life and death. Oncogene 27, S84-S92.
Shalem, O., Sanjana, N. E., Hartenian, E., Shi, X., Scott, D. a, Mikkelsen, T. S., Heckl, D., Ebert, B. L., Root, D. E., Doench, J. G., et al. (2014). Genome-scale CRISPR-Cas9 knockout screening in human cells. Science 343, 84-87.
Singhal, N., Graumann, J., Wu, G., Aranzo-Bravo, M. J., Han, D. W., Greber, B., Gentile, L., Mann, M., and Schöler, H. R. (2010). Chromatin-remodeling components of the baf complex facilitate reprogramming. Cell 141, 943-955.
Sokol, S. Y. (2011). Maintaining embryonic stem cell pluripotency with Wnt signaling. Development 138,4341-4350.
Takahashi, K., and Yamanaka, S. (2006). Induction of Pluripotent Stem Cells from Mouse Embryonic and Adult Fibroblast Cultures by Defined Factors. Cell 126, 663-676.
Takahashi, K., Tanabe, K., Ohnuki, M., Narita, M., Ichisaka, T., Tomoda, K., and Yamanaka, S. (2007). Induction of Pluripotent Stem Cells from Adult Human Fibroblasts by Defined Factors. Cell 131, 861-872.
Takashima, Y., Guo, G., Loos, R., Nichols, J., Ficz, G., Krueger, F., Oxley, D., Santos, F., Clarke, J., Mansfield, W., et al. (2015). Erratum: Resetting Transcription Factor Control Circuitry toward Ground-State Pluripotency in Human (Cell (2014) 158 (1254-1269)). Cell 162, 452-453.
Vetterkind, S., and Morgan, K. G. (2009). The pro-apoptotic protein Par-4 facilitates vascular contractility by cytoskeletal targeting of ZIPK. J. Cell. Mol. Med. 13, 887-895.
Wang, T., Birsoy, K., Hughes, N. W., Krupczak, M., Post, Y., Wei, J. J., Eric, S., and Sabatini, D. M. (2015). Identification and characterization of essential genes in the human genome. 13, 1-10.

Wang, W. P., Tzeng, T. Y., Wang, J. Y., Lee, D. C., Lin, Y. H., Wu, P. C., Chen, Y. P., Chiu, I. M., and Chi, Y. H. (2012). The EP300, KDM5A, KDM6A and KDM6B Chromatin Regulators Cooperate with KLF4 in the Transcriptional Activation of POU5F1. PLoS One 7.
Watanabe, K., Ueno, M., Kamiya, D., Nishiyama, A., Matsumura, M., Wataya, T., Takahashi, J. B., Nishikawa, S., Nishikawa, S. I., Muguruma, K., et al. (2007). A ROCK inhibitor permits survival of dissociated human embryonic stem cells. Nat. Biotechnol. 25,681-686.
Yamanaka, S. (2000). Essential role of NAT1/p97/DAP5 in embryonic differentiation and the retinoic acid pathway. EMBO J. 19, 5533-5541.
Yu, J., Vodyanik, M., Smuga-Otto, K., Antosiewicz-Bourget, J., Frane, J. L., Tian, S., Nie, J., Jonsdottir, G. a, Ruotti, V., Stewart, R., et al. (2007). Induced pluripotent stem cell lines derived from human somatic cells. Science (80-.). 318, 1917-1920.

Zhang, Y., Schulz, V. P., Reed, B. D., Wang, Z., Pan, X., Mariani, J., Euskirchen, G., Snyder, M. P., Vaccarino, F. M., Ivanova, N., et al. (2013). Functional genomic screen of human stem cell differentiation reveals pathways involved in neurodevelopment and neurodegeneration. Proc. Natl. Acad. Sci. U.S.A. 110, 12361-12366.
Zhao, M., Kim, P., Mitra, R., Zhao, J., and Zhao, Z. (2016). TSGene 2.0: An updated literature-based knowledgebase for Tumor Suppressor Genes. Nucleic Acids Res. 44, D1023-D1031.

What is claimed is:

1. A method of culturing, e.g., manufacturing, human pluripotent stem cells (hPSCs), comprising:
providing human pluripotent stem cells (hPSCs) (e.g., a cell population comprising hPSCs), e.g., human embryonic stem cells (hESCs) or induced pluripotent stem cells (iPSCs);
maintaining the hPSCs under conditions that allow for maintenance of pluripotency of the hPSCs;
contacting the hPSCs, with, one, two, three or more (e.g., all) of:
(i) an inhibitor of dissociation-induced death (DID), e.g., an inhibitor of a DID activator, e.g., one or more inhibitors of a DID activator disclosed in Table 7;
(ii) an inhibitor of stem cell pluripotency, e.g., one or more inhibitors of a target identified as OCT4 high as disclosed in Table 8;
(iii) a promoter of stem cell pluripotency, e.g., one or more activators of a target identified as OCT4 low as disclosed in Table 8;
(iv) an inhibitor of stem cell viability, e.g., cell divison, e.g., self-renewal, e.g., one or more inhibitors of a target identified as an enriched hPSC fitness gene as disclosed in Table 4; or
(v) an activator of stem cell viability, e.g., cell division, e.g., self-renewal, e.g., one or more activators of a target identified as a depleted hPSC fitness gene as disclosed in Table 4;
thereby culturing, e.g., manufacturing, the hPSCs.

2. A method of culturing, e.g., manufacturing, human pluripotent stem cells (hPSCs), comprising:
providing human pluripotent stem cells (hPSCs) (e.g., a population of hPSCs), e.g., human embryonic stem cell (hESCs) or induced pluripotent stem cells (iPSCs);
contacting the hPSCs with one, two, three or more (e.g., all) of:
(i) an inhibitor of dissociation-induced death (DID), e.g., an inhibitor of a DID activator, e.g., one or more inhibitors of a DID activator disclosed in Table 7;
(ii) an inhibitor of stem cell pluripotency, e.g., one or more inhibitors of a target identified as OCT4 high as disclosed in Table 8;
(iii) a promoter of stem cell pluripotency, e.g., one or more activators of a target identified as OCT4 low as disclosed in Table 8;
(iv) an inhibitor of stem cell viability, e.g., cell divison, e.g., self-renewal, e.g., one or more inhibitors of a target identified as an enriched hPSC fitness gene as disclosed in Table 4; or
(v) an activator of stem cell viability, e.g., cell division, e.g., self-renewal, e.g., one or more activators of a target identified as a depleted hPSC fitness gene as disclosed in Table 4;
under conditions that allow for:
a) growth of the hPSCs, e.g., about 2-, 5, 10-, 15- or 20-fold growth, e.g., as measured by an assay of Example 1;
b) preservation of viability of the hPSCs, e.g., as measured by an assay of Example 1; or
c) both of (a) and (b),
thereby culturing, e.g., manufacturing, the hPSCs.

3. The method of claim 1 or 2, wherein the DID inhibitor comprises one or more of a PAWR inhibitor or an inhibitor of a DID activator disclosed in Table 7.

4. The method of claim 3, wherein the DID inhibitor comprises an inhibitor of a DID activator disclosed in Table 7.

5. The method of claim 3 or 4, wherein the inhibitor of DID is chosen from: a low molecular weight compound; an antibody molecule; an RNAi targeting (e.g., siRNA or shRNA); an epigenetic modulator of; or a genetic modulator (e.g., a nuclease, e.g., a CRISPR/Cas9, a zinc-finger nuclease (ZFN), or a Transcription activator-like effector nuclease (TALEN)).

6. A method of culturing, e.g., manufacturing, human pluripotent stem cells (hPSCs), comprising:
providing human pluripotent stem cells (hPSCs) (e.g., a cell population comprising hPSCs), e.g., human embryonic stem cells (hESCs) or induced pluripotent stem cells (iPSCs);
maintaining the hPSCs under conditions that allow for maintenance of pluripotency of the hPSCs;
contacting the hPSCs, with an inhibitor of dissociation-induced death (DID), e.g., a PAWR inhibitor (e.g., a PAWR inhibitor described herein);
thereby culturing, e.g., manufacturing, the hPSCs.

7. A method of culturing, e.g., manufacturing, human pluripotent stem cells (hPSCs), comprising:
providing human pluripotent stem cells (hPSCs) (e.g., a population of hPSCs), e.g., human embryonic stem cell (hESCs) or induced pluripotent stem cells (iPSCs);
contacting the hPSCs with an inhibitor of dissociation-induced death (DID), e.g., a PAWR inhibitor (e.g., a PAWR inhibitor described herein), under conditions that allow for:
i) growth of the hPSCs, e.g., about 2-, 5, 10-, 15- or 20-fold growth, e.g., as measured by an assay of Example 1;
ii) preservation of viability of the hPSCs, e.g., as measured by an assay of Example 1; or
iii) both of (i) and (ii),
thereby culturing, e.g., manufacturing, the hPSCs.

8. The method of any of claim 3 or 6-7, wherein the DID inhibitor comprises a PAWR inhibitor, e.g., as described herein.

9. The method of any of claim 3 or 6-8, wherein the PAWR inhibitor is chosen from: a low molecular weight compound inhibitor of PAWR; an anti-PAWR antibody molecule; an RNAi targeting PAWR (e.g., siRNA or shRNA); an epigenetic modulator of PAWR; or a genetic modulator of PAWR (e.g., a nuclease targeting PAWR, e.g., a CRISPR/Cas9, a zinc-finger nuclease (ZFN), or a Transcription activator-like effector nuclease (TALEN) targeting PAWR).

10. The method of any of claims 6-9, wherein the PAWR inhibitor, is administered at a dose that results in reduced, e.g., lesser, dissociation-induced death of hPSCs as measured by an assay of Example 1.

11. The method of any of claims 6-10, wherein the PAWR inhibitor is provided at a dose that reduces, e.g., inhibits, DID by at least 10%, 20%, 30%, 40%, 50%, 60%, 70%, 80%, 90%, 95%, 99%, or 100%, e.g., compared to the hPSCs in the absence of the PAWR inhibitor.

12. The method of any one of claims 6-11, wherein the PAWR inhibitor reduces membrane blebbing, e.g., as measured by an assay of Example 1 by at least 10%, 20%, 30%, 40%, 50%, 60%, 70%, 80%, 90%, 95%, 99%, or 100%, e.g., compared to the hPSCs in the absence of the PAWR inhibitor.

13. The method of any one of claims 6-12, wherein the PAWR inhibitor increases one or more of survival, proliferation, expansion of the hPSCs by at least 10%, 20%, 30%, 40%, 50%, 60%, 70%, 80%, 90%, 95%, 99%, or 100%, e.g., compared to the hPSCs in the absence of the PAWR inhibitor.

14. The method of any one of claims 6-13, wherein the PAWR inhibitor increases the ability to passage the hPSCs for a first, second, third, or more passages.

15. The method of claim 14, wherein the PAWR inhibitor increases one or more of: survival, proliferation, or expansion of the hPSCs after one or more passages by at 10%, 20%, 30%, 40%, 50%, 60%, 70%, 80%, 90%, 95%, 99%, or 100%, e.g., compared to the hPSCs in the absence of the PAWR inhibitor.

16. The method of any one of claims 6-15, wherein the PAWR inhibitor increases the survival of hPSCs as single cells by at least 10%, 20%, 30%, 40%, 50%, 60%, 70%, 80%, 90%, 95%, 99%, or 100%, e.g., compared to the hPSCs in the absence of the PAWR inhibitor.

17. The method of any of claims 6-16, wherein the method further comprises contacting the population of cells with an additional, e.g., a second or third, DID inhibitor chosen from a Rho-dependent protein kinase (ROCK) inhibitor (e.g., a ROCK inhibitor described herein, e.g., a ROCK1 inhibitor, or a ROCK2 inhibitor, or both) or a Myosin inhibitor (e.g., a Myosin inhibitor described herein).

18. The method of claim 17, wherein the DID inhibitor comprises a ROCK inhibitor, e.g., Y-27632 or Thiazovivin.

19. The method of claim 18, wherein the DID inhibitor comprises a Myosin inhibitor, e.g., blebbistatin.

20. The method of any of claims 1-19, wherein the hPSCs are maintained under conditions that result in one, two, three, or all of the following:
i) a cell density in the range of 0.5×10⁵ to 5×10⁵;
ii) a culture size of at least 1000 cm², 2000 cm², 3000 cm², 4000 cm², or 5000 cm²;
iii) at least 10, 15, 20, 25, 30, 25, 40, 45, 50, 55, 60, 65, 70, 75, 80, 90 or 100 million cells; or
iv) viability of the cells, as measured by an assay of Example 1.

21. The cells produced by a method of any of claims 1-20, wherein the cells maintain at least 5%, 10%, 20%, 30%, 40%, 50%, 60%, 70%, 80%, 90% or more viable cells after a freeze-thawing cycle, e.g., after one or more cycles of freezing and/or thawing.

22. The method of any of claims 1-21, further comprising modifying the hPSCs by contacting the hPSCs with an exogenous or overexpressed molecule, e.g., a nucleotide encoding a target protein, e.g., a target protein described herein, under conditions that allow for expression of the target protein, thereby making a modified population of hPSCs.

23. The method of claim 22, wherein the exogenous or overexpressed molecule comprises a nucleic acid (e.g., RNA, e.g., mRNA, miRNA, or siRNA) or a protein.

24. The method of claim 22 or 23, wherein the exogenous molecule does not naturally exist in the hPSCs.

25. The method of claim 22 or 23, wherein the exogenous molecule induces differentiation of the hPSC into a differentiated cell, e.g., a differentiated cell described herein, e.g., a lineage committed cell, e.g., a cardiomyocyte.

26. The method of claim 25, further comprising monitoring the state of differentiation of the hPSCs by measuring the level of a marker, e.g., a biomarker, wherein the marker level is indicative of a particular differentiated cell, e.g., a lineage committed cell.

27. The method of any of claims 22-26, wherein the target protein is Wnt3a and the modified hPSCs express Wnt3a.

28. The method of any of claims 1-27, further comprising modifying the hPSCs by contacting the hPSCs with an exogenous molecule that reduces the level, e.g., amount or expression, of an endogenous target in the hPSCs.

29. The method of any of claims 1-28, wherein the hPSCs are derived from cultured cells, e.g., a cell line, e.g., H1-hESC cell line.

30. The method of any of claims 1-29, wherein the hPSCs are autologous to a subject, e.g., a subject to be treated.

31. The method of any of claims 1-30, wherein the providing of human pluripotent stem cells comprises obtaining stem cells from a subject.

32. The method of any of claims 1-31, further comprising freezing the hPSCs, e.g., under conditions that maintain viability of the hPSCs, e.g., at least 5%, 10%, 20%, 30%, 40%, 50%, 60%, 70%, 80%, 90% or more viable cells.

33. The method of any of claims 1-32, further comprising storing the hPSCs under conditions suitable for transport, e.g., to a recipient entity, e.g., a laboratory, a hospital, a health care provider.

34. A human pluripotent stem cells (hPSC) (e.g., a population of hPSCs), e.g., human embryonic stem cells (hESCs) or induced pluripotent stem cells (iPSCs) cultured by the method of any of claims 1-33.

35. A frozen preparation of hPSCs, comprising hPSCs cultured by the method of claim 34.

36. A method of treating a condition, e.g., a condition described herein (e.g., a condition associated with expression of a target protein), in a subject, comprising:
providing hPSCs received from a provider entity, e.g., wherein said provider entity has received the hPSCs cultured according to the method of any of claims 1-33, or has cultured the HPCs according to the method of any of claims 1-33;
administering the hPSCs to the subject,
thereby treating the condition.

37. A method of treating a condition, e.g., a condition described herein (e.g., a condition associated with expression of a target protein), in a subject, comprising:
administering the hPSCs to the subject, wherein the hPSCs are cultured, or have been cultured, according to the method of any of claims 1-33;
thereby treating the condition.

38. A method of treating a condition, e.g., a condition described herein (e.g., a condition associated with a target protein), in a subject, e.g., a subject described herein, comprising administering the hPSCs cultured according to the method of any of claims 1-33, to the subject, thereby treating the condition.

39. A composition comprising a hPSCs for use in a method of treating a condition, e.g., a condition described herein (e.g., a condition associated with expression of a target protein), in a subject, wherein the method comprises administering the hPSCs cultured according to the method of any of claims 1-33, to the subject, thereby treating the condition.

40. The method of claim 37 or 38, or the composition for use of claim 39, wherein the condition is a cardiac condition, e.g., a heart disease, e.g., myocardial infarction.

41. The method or composition for use of claim 40, wherein the target protein is Wnt3a and the hPSCs are modified to express Wnt3a.

42. The hPSCs of claims 32, or the method of claim 35, further comprising thawing and preparing the hPSCs for administration into the subject.

43. The method of any of claims 36-42, wherein the hPSCs are administered in one or more, e.g., two, three, four or more, administrations to the subject.

44. The method of any of claims 36-43, wherein the hPSCs are administered in repeated administrations over a specified period of time, e.g., as described herein.

45. The method of any of claims 36-44, wherein the hPSCs are administered by intravenous, intramuscular administration, or by implantation.

46. A method of reducing, e.g., inhibiting, dissociation-induced death (DID) in a population of hPSCs, comprising contacting the population of hPSCs, with an inhibitor of DID, e.g., an inhibitor of a DID activator, e.g., one or more inhibitors of a DID activator disclosed in Table 7, e.g., a PAWR inhibitor (e.g., a PAWR inhibitor described herein).

47. The method of claim 46, wherein the hPSC is cultured, e.g., manufactured, using a method of any of claims 1-33.

48. The method of claim 46 or 47, wherein the method further comprises contacting the population of cells with a ROCK inhibitor, e.g., Y-27632 or Thiazovivin.

49. The method of any of claims 46-48, wherein the method further comprises contacting the population of cells with a Myosin inhibitor, e.g., blebbistatin.

50. The method of any of claims 46-49, wherein the level of DID is measured by an assay of Example 1.

51. The method of any of claims 46-50, wherein the level of DID is reduced, e.g., inhibited, compared to a population of cells cultured without the inhibitor of DID e.g., the inhibitor of a DID activator, e.g., one or more inhibitors of a DID activator disclosed in Table 7, e.g., a PAWR inhibitor.

52. The method of any of claims 46-51, wherein the PAWR inhibitor reduces membrane blebbing as measured by an assay of Example 1 by at least 10%, 20%, 30%, 40%, 50%, 60%, 70%, 80%, 90%, 95%, 99%, or 100%, e.g., compared to the hPSCs in the absence of the PAWR inhibitor.

53. The method of any one of claims 46-52, wherein the PAWR inhibitor increases one or more of survival, proliferation, expansion of the hPSCs by at least 10%, 20%, 30%, 40%, 50%, 60%, 70%, 80%, 90%, 95%, 99%, or 100%, e.g., compared to the hPSCs in the absence of the PAWR inhibitor.

54. The method of any one of claims 46-53, wherein the PAWR inhibitor increases the ability to passage the hPSCs for a first, second, third, or more passages.

55. The method of claim 54, wherein the PAWR inhibitor increases one or more of: survival, proliferation, or expansion of the hPSCs after one or more passages by at 10%, 20%, 30%, 40%, 50%, 60%, 70%, 80%, 90%, 95%, 99%, or 100%, e.g., compared to the hPSCs in the absence of the PAWR inhibitor.

56. The method of any one of claims 46-55, wherein the PAWR inhibitor increases the survival of hPSCs as single cells by at least 10%, 20%, 30%, 40%, 50%, 60%, 70%, 80%, 90%, 95%, 99%, or 100%, e.g., compared to the hPSCs in the absence of the PAWR inhibitor.

57. The method of any of claims 46-56, wherein the method further comprises contacting the population of cells with an additional, e.g., a second or third, DID inhibitor, e.g., a Rho-dependent protein kinase (ROCK) inhibitor, e.g., a ROCK inhibitor described herein, e.g., a ROCK1 inhibitor, or a ROCK2 inhibitor, or both.

58. The method of claim 57, wherein the ROCK inhibitor comprises Y-27632 or Thiazovivin.

59. A method of modifying a human pluripotent stem cell (hPSC), e.g., a population of hPSCs; comprising contacting the hPSCs with a DID inhibitor, e.g., an inhibitor of a DID activator, e.g., one or more inhibitors of a DID activator disclosed in Table 7, e.g., a PAWR inhibitor (e.g., a PAWR inhibitor described herein), and an additional agent that modifies the hPSCs.

60. The method of claim 59, wherein the additional agent comprises an exogenous or overexpressed molecule, e.g., a nucleotide encoding a target protein, e.g., a target protein described herein, under conditions that allow for expression of the target protein.

61. The method of claim 60, wherein the exogenous or overexpressed molecule comprises a nucleic acid (e.g., RNA, e.g., mRNA, miRNA, or siRNA) or a protein.

62. The method of claim 60 or 61, wherein the exogenous molecule does not naturally exist in the hPSCs.

63. The method of any of claims 60-62, wherein the exogenous molecule induces differentiation of the hPSC into a differentiated cell, e.g., a differentiated cell described herein, e.g., a lineage committed cell, e.g., a cardiomyocyte.

64. The method of claim 63, further comprising monitoring the state of differentiation of the hPSCs by measuring the level of a marker, e.g., a biomarker, wherein the marker level is indicative of a particular differentiated cell, e.g., a lineage committed cell.

65. The method of any of claims 60-64, wherein the target protein is Wnt3a and the modified hPSCs express Wnt3a.

66. The method of claim 59, wherein the additional agent comprises an exogenous molecule that modifies the hPSCs by reducing the level, e.g., amount or expression, of an endogenous target in the hPSCs.

67. The method of any of claims 59-67, wherein the hPSCs are derived from cultured cells, e.g., a cell line, e.g., H1-hESC cell line.

68. The method of any of claims 59-67, wherein the hPSCs are autologous to a subject, e.g., a subject to be treated.

69. The method of any of claims 59-68, wherein the hPSC is cultured, e.g., manufactured, using a method of any of claims 1-33.

70. The method of any of claims 59-69, further comprising freezing the hPSCs, e.g., under conditions that maintain viability of the hPSCs, e.g., at least 5%, 10%, 20%, 30%, 40%, 50%, 60%, 70%, 80%, 90% or more viable cells.

71. A composition comprising:
a) a population of human pluripotent stem cells (hPSCs), e.g., human embryonic stem cells (hESCs) or induced pluripotent stem cells (iPSCs);.
b) a culture medium;
c) an hPSC regulator, e.g., one, two, three or more (e.g., all) of:
(i) an inhibitor of dissociation-induced death (DID), e.g., an inhibitor of a DID activator, e.g., one or more inhibitors of a DID activator disclosed in Table 7;
(ii) an inhibitor of stem cell pluripotency, e.g., one or more inhibitors of a target identified as OCT4 high as disclosed in Table 8;
(iii) a promoter of stem cell pluripotency, e.g., one or more activators of a target identified as OCT4 low as disclosed in Table 8;
(iv) an inhibitor of stem cell viability, e.g., cell divison, e.g., self-renewal, e.g., one or more inhibitors of a target identified as an enriched hPSC fitness gene as disclosed in Table 4, or
(v) an activator of stem cell viability, e.g., cell division, e.g., self-renewal, e.g., one or more activators of a target identified as a depleted hPSC fitness gene as disclosed in Table 4; and
d) optionally, a ROCK inhibitor, e.g., a ROCK inhibitor described herein.

72. A composition comprising:
a) a population of human pluripotent stem cells (hPSCs), e.g., human embryonic stem cells (hESCs) or induced pluripotent stem cells (iPSCs);
b) a culture medium;
c) a molecule to modify the hPSCs, e.g., an exogenous or overexpressed molecule;
d) an hPSC regulator, e.g., one, two, three or more (e.g., all) of:
(i) an inhibitor of dissociation-induced death (DID), e.g., an inhibitor of a DID activator, e.g., one or more inhibitors of a DID activator disclosed in Table 7;
(ii) an inhibitor of stem cell pluripotency, e.g., one or more inhibitors of a target identified as OCT4 high as disclosed in Table 8;
(iii) a promoter of stem cell pluripotency, e.g., one or more activators of a target identified as OCT4 low as disclosed in Table 8;
(iv) an inhibitor of stem cell viability, e.g., cell divison, e.g., self-renewal, e.g., one or more inhibitors of a target identified as an enriched hPSC fitness gene as disclosed in Table 4, or
(v) an activator of stem cell viability, e.g., cell division, e.g., self-renewal, e.g., one or more activators of a target identified as a depleted hPSC fitness gene as disclosed in Table 4; and
e) optionally, an additional dissociation-induced death inhibitor, e.g., a ROCK inhibitor, e.g., a ROCK inhibitor described herein.

73. The composition of claim 71 or 72, wherein the DID inhibitor comprises one or more of a PAWR inhibitor, or an inhibitor of a DID activator disclosed in Table 7.

74. The compositions of claim 73, wherein the DID inhibitor comprises an inhibitor of a DID activator disclosed in Table 7.

75. The compositions of claim 73 or 74, wherein the DID inhibitor is chosen from: a low molecular weight compound; an antibody molecule; an RNAi targeting (e.g., siRNA or shRNA); an epigenetic modulator of; or a genetic modulator (e.g., a nuclease, e.g., a CRISPR/Cas9, a zinc-finger nuclease (ZFN), or a Transcription activator-like effector nuclease (TALEN)).

76. A composition comprising:
i) a population of human pluripotent stem cells (hPSCs), e.g., human embryonic stem cells (hESCs) or induced pluripotent stem cells (iPSCs);.
ii) a culture medium;
iii) a dissociation-induced death (DID) inhibitor, e.g., a PAWR inhibitor (e.g., a PAWR inhibitor described herein); and
iv) optionally, a ROCK inhibitor, e.g., a ROCK inhibitor described herein.

77. A composition comprising:
i) a population of human pluripotent stem cells (hPSCs), e.g., human embryonic stem cells (hESCs) or induced pluripotent stem cells (iPSCs);
ii) a culture medium;
iii) a molecule to modify the hPSCs, e.g., an exogenous or overexpressed molecule;
iv) a dissociation-induced death (DID) inhibitor, e.g., a PAWR inhibitor (e.g., a PAWR inhibitor described herein); and
v) optionally, an additional dissociation-induced death inhibitor, e.g., a ROCK inhibitor, e.g., a ROCK inhibitor described herein.

78. The composition of any of claim 73 or 76-77, wherein the DID inhibitor comprises a PAWR inhibitor, e.g., as described herein.

79. The composition of any of claim 73 or 76-78, wherein the PAWR inhibitor, is provided at a dose that results in reduced, e.g., lesser, dissociation-induced death of hPSCs as measured by an assay of Example 1.

80. The composition of any of claim 73, or 76-79, wherein the PAWR inhibitor is provided at a dose that reduces, e.g., inhibits, DID by at least 10%, 20%, 30%, 40%, 50%, 60%, 70%, 80%, 90%, 95%, 99%, or 100%, e.g., compared to the hPSCs in the absence of the PAWR inhibitor.

81. The composition of any of claim 73, or 76-80, wherein the culture medium comprises TeSR-E8 media, e.g., E8 media, comprising: about 50 mg/mL of G418, about 50 mg/uL of Doxcycyline, and about 1 mg/uL of Puromycin.

82. The composition of claim 81, wherein the E8 media further comprise about 10,000 U/mL of Penicillin-Streptomycin.

83. A method of selecting a degron, e.g., a method of screening for a degron, comprising:
providing human pluripotent stem cells (hPSCs), e.g., a population of hPSCs, modified, e.g., by a method described herein, to express a fusion protein comprising a candidate degron and PAWR (e.g., a fragment of PAWR or full-length PAWR); and
selecting the candidate degron when the fusion protein decreases dissociation-induced death (DID) of the hPSCs, as compared to a population of hPSCs not expressing the degron.

84. A method of selecting a compound that regulates a degron, e.g., a method of screening for a compound that regulates a degron, comprising:
providing human pluripotent stem cells (hPSCs), e.g., a population of hPSCs, modified, e.g., by a method described herein, to express a fusion protein comprising a degron and PAWR;
treating the hPSCs with a candidate compound that regulates the degron; and
selecting the compound when treatment of the compound inreases or decreases dissociation-induced death (DID) of the hPSCs, as compared to a population of hPSCs not treated with the compound.

85. The method of claim 83 or 84, wherein the hPSCs are cultured, e.g., manufactured, by the method of any one of claims 1-33.

86. The method of any of claims 83-85, wherein the candidate degron is chosen from a furin degron (FurON) domain; a degron derived from an FKB protein (FKBP); a degron derived from dihydrofolate reductase (DHFR); a degron derived from an estrogen receptor (ER); a degron derived from an Ikaros family of transcription factors (e.g., IKZF1, or IKZF3); or a degron derived from a protein listed in Table 21 of International Application WO 2017/181119.

87. The method of any of claims 83-86, wherein the hPSCs expressing a fusion protein comprising a candidate degron can be cultured in the presence or absence of a stabilization compound, e.g., as described herein.

88. The method of any of claims 83-87, wherein the modified hPSCs expressing a fusion protein comprising a candidate degron cultured in the absence of a stabilization compound, e.g., as described herein, have a decrease in DID as compared to modified hPSCs expressing a fusion protein comprising a candidate degron cultured in the presence of a stabilization compound.

89. The method of any of claims 83-88, wherein a decrease in DID in the modified hPSCs is due to degradation of PAWR by the degron, e.g., by targeting PAWR for proteasomal degradation.

90. The method of any of claims 83-89, wherein the hPSCs are modified to express a fusion protein by contacting the population of hPSCs with a nucleotide encoding the fusion protein comprising the candidate degron and PAWR (e.g., a fragment of PAWR or full-length PAWR), under conditions that allow for expression of the fusion protein.

91. The method of any of claims 83-90, wherein the hPSCs are modified to express a fusion protein by contacting the population of hPSCs with a nucleotide encoding a fusion protein, e.g., a plurality of nucleotides encoding distinct (e.g., non-identical) fusion proteins, e.g., a library of fusion proteins, wherein each fusion protein comprising the library of fusion proteins comprises a distinct degron (e.g., a non-identical degron) and PAWR (e.g., a fragment of PAWR or full-length PAWR).

92. The method of any of claims 83-91, wherein the fusion protein further comprises a protease cleavage site, e.g., a furin cleavage site.

93. The method of any of claims 83-92, wherein the fusion protein further comprises a tag e.g., a unique identifier tag, e.g., a unique nucleotide tag comprising at least 3, 4, 5, 6, 7, 8, 9, 10, 15, 20, or 30 nucleotides.

94. The method of claim 93, wherein the tag is used in the identification of a candidate degron obtained from the method of any of claims 83-92.

95. The method of any of claims 83-94, wherein the hPSCs have been previously modified to not express endogenous PAWR, e.g., by a method described herein, e.g., CRISPR/Cas9.

96. The method of claim any of claims 83-95, wherein DID is measured by an assay of Example 1.


SEQUENCE LISTING (24 sequences)

SEQ ID NO: 1 - DNA, 20 bp, Artificial Sequence
  source: /note=“Description of Artificial Sequence Synthetic oligonucleotide”
tcgagtgtgctactcaactc

SEQ ID NO: 2 - DNA, 20 bp, Artificial Sequence
  source: /note=“Description of Artificial Sequence Synthetic oligonucleotide”
cgagctcaacaacaacctcc

SEQ ID NO: 3 - DNA, 20 bp, Artificial Sequence
  source: /note=“Description of Artificial Sequence Synthetic oligonucleotide”
gaagggacagaagatgacag

SEQ ID NO: 4 - DNA, 20 bp, Artificial Sequence
  source: /note=“Description of Artificial Sequence Synthetic oligonucleotide”
gaagggacagaagatgacag

SEQ ID NO: 5 - DNA, 20 bp, Artificial Sequence
  source: /note=“Description of Artificial Sequence Synthetic oligonucleotide”
gagcgctgctcagatagcga

SEQ ID NO: 6 - DNA, 20 bp, Artificial Sequence
  source: /note=“Description of Artificial Sequence Synthetic oligonucleotide”
aatggcgggctgcatccagg

SEQ ID NO: 7 - DNA, 21 bp, Artificial Sequence
  source: /note=“Description of Artificial Sequence Synthetic oligonucleotide”
tccactgggccgaagaggcgg

SEQ ID NO: 8 - DNA, 20 bp, Artificial Sequence
  source: /note=“Description of Artificial Sequence Synthetic oligonucleotide”
ggcgccatgtcagaaccggc

SEQ ID NO: 9 - DNA, 20 bp, Artificial Sequence
  source: /note=“Description of Artificial Sequence Synthetic oligonucleotide”
tttgggaatatggcgaccgg

SEQ ID NO: 10 - DNA, 20 bp, Artificial Sequence
  source: /note=“Description of Artificial Sequence Synthetic oligonucleotide”
ggtggctaccggaccagcag

SEQ ID NO: 11 - DNA, 20 bp, Artificial Sequence
  source: /note=“Description of Artificial Sequence Synthetic oligonucleotide”
cgagctcaacaacaacctcc

SEQ ID NO: 12 - DNA, 20 bp, Artificial Sequence
  source: /note=“Description of Artificial Sequence Synthetic oligonucleotide”
gaagtgatggaagatcacgc

SEQ ID NO: 13 - DNA, 20 bp, Artificial Sequence
  source: /note=“Description of Artificial Sequence Synthetic oligonucleotide”
tggtagtggactcactgcac

SEQ ID NO: 14 - DNA, 20 bp, Artificial Sequence
  source: /note=“Description of Artificial Sequence Synthetic oligonucleotide”
gcagcaccgtggctaccgca

SEQ ID NO: 15 - DNA, 20 bp, Artificial Sequence
  source: /note=“Description of Artificial Sequence Synthetic oligonucleotide”
atgaagagagccgaagtcag

SEQ ID NO: 16 - DNA, 20 bp, Artificial Sequence
  source: /note=“Description of Artificial Sequence Synthetic oligonucleotide”
ggaatctgtctttctggagg

SEQ ID NO: 17 - DNA, 20 bp, Artificial Sequence
  source: /note=“Description of Artificial Sequence Synthetic oligonucleotide”
aaggactgacctccaagccc

SEQ ID NO: 18 - DNA, 20 bp, Artificial Sequence
  source: /note=“Description of Artificial Sequence Synthetic oligonucleotide”
tagaaagctggggccttgga

SEQ ID NO: 19 - DNA, 20 bp, Artificial Sequence
  source: /note=“Description of Artificial Sequence Synthetic oligonucleotide”
cgccttcctatttcaggtaa

SEQ ID NO: 20 - DNA, 20 bp, Artificial Sequence
  source: /note=“Description of Artificial Sequence Synthetic oligonucleotide”
cagtctgtgaaaggacgctg

SEQ ID NO: 21 - DNA, 20 bp, Artificial Sequence
  source: /note=“Description of Artificial Sequence Synthetic oligonucleotide”
ggcgcgctcgaggactccaa

SEQ ID NO: 22 - DNA, 20 bp, Artificial Sequence
  source: /note=“Description of Artificial Sequence Synthetic oligonucleotide”
gttgcagggtggggacccgg

SEQ ID NO: 23 - DNA, 20 bp, Artificial Sequence
  source: /note=“Description of Artificial Sequence Synthetic oligonucleotide”
gctggccggtagtgactggt

SEQ ID NO: 24 - DNA, 20 bp, Artificial Sequence
  source: /note=“Description of Artificial Sequence Synthetic oligonucleotide”
ggctgctggccggtagtgac